(19) United States
(12) Patent Application Publication
(10) Pub. No.: US 2008/0096903 A1
(43) Pub. Date: Apr. 24, 2008

(54) SULFAMOYL-CONTAINING DERIVATIVES AND USES THEREOF

(75) Inventors: Jianxin Chen, Littleton, MA (US); Gerhard Sperl, Salem, NH (US); Vincent Gullo, Basking Ridge, NJ (US); Lalitha Sista, Moula Ali (IN); Dallas Hughes, Milford, MA (US); Yucai Peng, Shrewsbury, MA (US); William Pierceall, Sudbury, MA (US); Andrew Weiskopf, Needham, MA (US); Jeremy Ian Levin, New City, NY (US); Russell George Dushin, Garrison, NY (US); Mercy Otteng, Pearl River, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/875,320

(22) Filed: Oct. 19, 2007

Related U.S. Application Data
(60) Provisional application No. 60/852,880, filed on Oct. 19, 2006.

(51) Int. Cl. A61K 31/52; C07D 207/00; C07D 235/04; C07D 239/00; C07D 249/16; C07D 295/22; C07D 471/02; C07D 473/00; C07D 487/00; C07D 498/02

(52) U.S. Cl. 514/263.1; 544/254; 544/262; 544/277; 544/280; 546/113; 546/117; 546/118; 548/257; 548/304.7; 548/400; 548/541; 548/542; 548/544

(57) ABSTRACT

Sulfamoyl-containing compounds are disclosed, having utility as inhibitors of disease-related targets, such as Heat Shock Protein 90 (HSP90), and which are useful for treating disorders, e.g., proliferative disorders, including HSP90-mediated disorders. Methods for preparing and using the disclosed compounds are also described.

### CROSS-REFERENCE TO RELATED APPLICATIONS

This application claims the benefit under 35 U.S.C. § 119(e) to U.S. Provisional Patent Application No. 60/852,880, filed Oct. 19, 2006, which is hereby incorporated by reference in its entirety.

### FIELD OF THE INVENTION

The present invention relates to heterocyclic ring systems derivatives comprising sulfamoyl and other substituents, the preparation of these and uses of such compounds in the treatment of proliferative and other diseases.

### BACKGROUND OF THE INVENTION

Heat shock protein 90 (HSP90) is a family of ubiquitous chaperone proteins that are involved in folding, activation and assembly of a wide range of proteins, such as key proteins involved in signal transduction, cell cycle control and transcriptional regulation. HSP90 has recently been shown to been implicated in cellular proliferation. For example, HSP90 α and β proteins are associated with important signaling polypeptides associated with cancer (such as steroid hormone receptors and protein kinases, e.g., Raf-1, EGFR, v-Src family kinases, Cdk4, and ErbB-2 (See, for example, Buchner J. TIBS 24, 136-141 (1999); Stepanova, L. et al. Genes Dev., 10, 1491-502 (1996); and Dai, K. et al. J. Biol. Chem., 271, 22030-22034 (1996)). For a review of HSP90 see Chiosis et al., Drug Discovery Today, Vol. 9, pp. 881-888 (2004).

Certain antibiotics, for example, herbimycin A (HA), geldanamycin (GM), and 17-allylaminogeldanamycin (17-AAG) are believed to exert anticancerous effects by binding to a highly conserved N-terminus ATP binding pocket of HSP90, thereby competing with substrates that would otherwise bind to HSP90 (Stebbins, C. et al. Cell, 89, 239-250 (1997)). In vitro and in vivo studies have demonstrated that occupancy of this N-terminal pocket by ansamycins and other HSP90 inhibitors alters HSP90 function and inhibits protein folding. It is also known that HSP90 substrate destabilization occurs in both tumor and non-transformed cells and has been shown to be especially effective on a subset of signaling regulators, e.g., Raf (Schulte, T. W. et al. Biochem. Biophys. Res. Commun. 1997, 239, 655-9; Schulte, T. W., et al. J. Biol. Chem. 1995, 270, 24585-8), nuclear steroid receptors (Segnitz, B.; U. Gehring J. Biol. Chem. 1997, 272, 18694-18701; Smith, D. F. et al. Mol. Cell. Biol. 1995, 15, 6804-12), v-Src (Whitesell, L., et al. Proc. Natl. Acad. Sci. USA 1994, 91, 8324-8328) and certain transmembrane tyrosine kinases (Sepp-Lorenzino, L. et al. J. Biol. Chem. 1995, 270, 16580-16587) such as EGF receptor (EGFR) and HER2/Neu (Hartmann, F., et al. Int. J. Cancer 1997, 70, 221-9; Miller, P. et al. Cancer Res. 1994, 54, 2724-2730; Mimnaugh, E. G., et al. J. Biol. Chem. 1996, 271, 22796-801; Schnur, R. et al. J. Med. Chem. 1995, 38, 3806-3812), CDK4, and mutant p53. Erlichman et al. Proc. AACR 2001, 42, abstract 4474. The ansamycin-induced loss of these proteins leads to the selective disruption of certain regulatory pathways and results in growth arrest at specific phases of the cell cycle (Muise-Heimericks, R. C. et al. J. Biol. Chem. 1998, 273, 29864-72), and apoptosis, and/or differentiation of cells so treated (Vasilevskaya, A. et al. Cancer Res., 1999, 59, 3935-40). Ansamycins thus hold great promise for the treatment and/or prevention of many types of cancers and proliferative disorders, and also hold promise as traditional antibiotics. However, their relative insolubility makes them difficult to formulate and administer, they are not easily synthesized and currently must, at least in part, be generated through fermentation and are severely limited in dosing by their toxicity.

HSP90 inhibitors have also been implicated in other functions, including use as anti-inflammation agents, anti-infectious disease agents, agents for treating autoimmunity, agents for treating stroke, ischemia, multiple sclerosis, cardiac disorders, central nervous system related disorders and agents useful in promoting nerve regeneration (See, e.g., Rosen et al. WO 02/09696 (PCT/US01/23640); Degranco et al. WO 99/51223 (PCT/US99/07242); Gold, U.S. Pat. No. 6,210,974 Bl; DeFranco et al., U.S. Pat. No. 6,174,875. Overlapping somewhat with the above, there are reports in the literature that fibrogenetic disorders including but not limited to scleroderma, polymyositis, systemic lupus, rheumatoid arthritis, liver cirrhosis, keloid formation, interstitial nephritis, and pulmonary fibrosis also may be treatable with HSP90 inhibitors. Strehlow, WO 02/02123 (PCT/US01/20578). Still further HSP90 modulation, modulators and uses thereof are reported in Application Nos. PCT/US03/04283, PCT/US02/35938, PCT/US02/16287, PCT/US02/06518, PCT/US98/09805, PCT/US00/09512, PCT/US01/09512, PCT/US01/23640, PCT/US01/46303, PCT/US01/46304, PCT/US02/06518, PCT/US02/29715, PCT/US02/35069, PCT/US02/35938, PCT/US02/39993, 60/293,246, 60/371,668, 60/335,391, 60/128,593, 60/337,919, 60/340,762, 60/359,484 and 60/331,893.

Recently, purine derivatives showing moderate HSP90 inhibitory activity have been reported, e.g., in PCT/US02/35069; PCT/US02/36075. Purine moieties are well accepted bioisosteres for a variety of ATP-dependent molecular targets, see, JP 10025294; U.S. Pat. No. 4,748,177; U.S. Pat. No. 4,772,606; U.S. Pat. No. 6,369,092; Chiosis and Rosen, U.S. Pub. 2004/0102458, Kasibhatla et al., U.S. Pub. 2005/0049263, WO 00/06573; WO 02/055521; WO 02/055082; WO 02/055083; European Patent 0178178; Eur. J. Med. Chem. 1994, 29(1), 3-9; and J. Het. Chem. 1990, 27(5), 1409. In addition, adenine derivatives with activity against HSP90 have been reported. (See Chiosis et al, WO 2006/084030) However, there remains a need for novel and potent HSP90 inhibitors that meet the demanding biological and pharmaceutical criteria required to justify the time and expense of human clinical trials.

### SUMMARY OF THE INVENTION

In one aspect, the present invention relates to novel organic compounds, such as heterocyclic ring systems containing a sulfamoyl appendage, i.e. derivatives of adenine and other purines that bind to targets such as HSP90, for example, HSP90-α, and are therefore useful in treating or preventing proliferative and other disorders. These agents may also function as protein modulators for proteins found in cancer and other cells.

In another aspect, the present invention relates to novel organic compounds useful in treating or preventing cancer arising in animals or human patients and having the general structure of Formula I to Formula XXVIII: 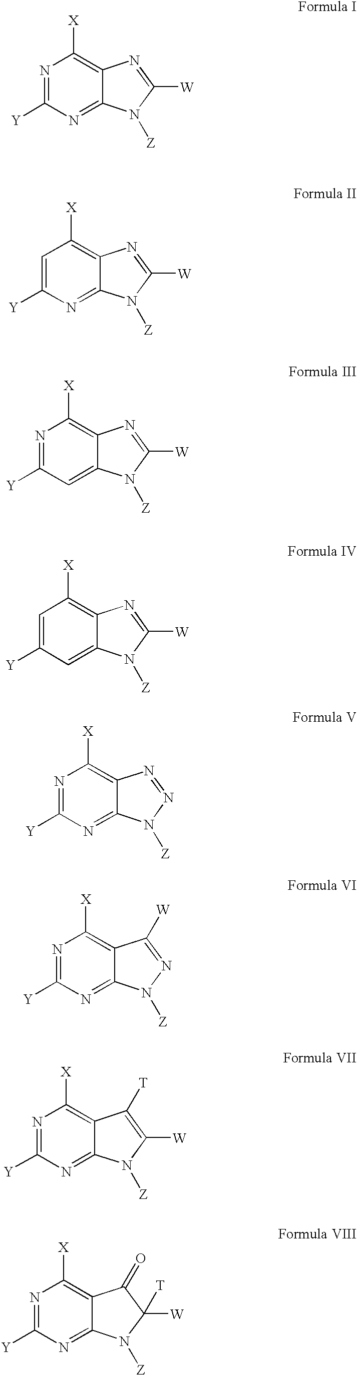 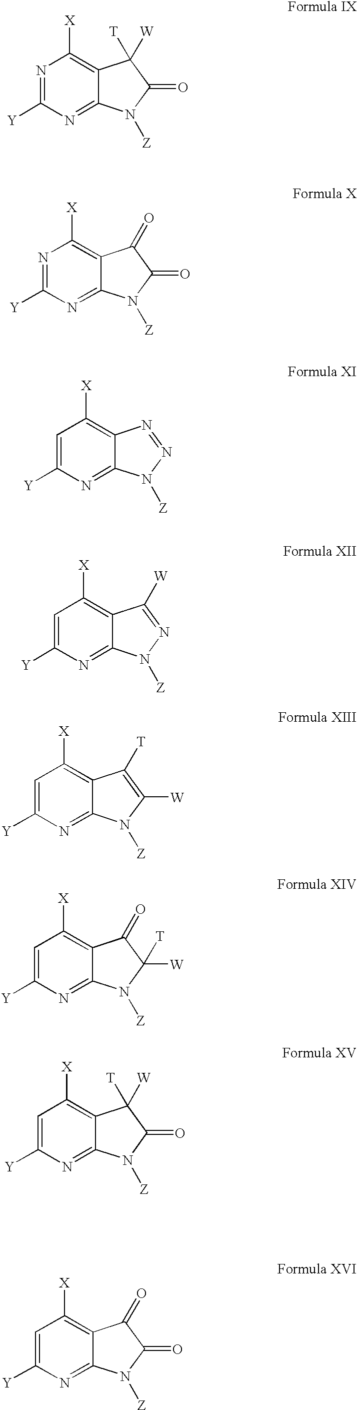 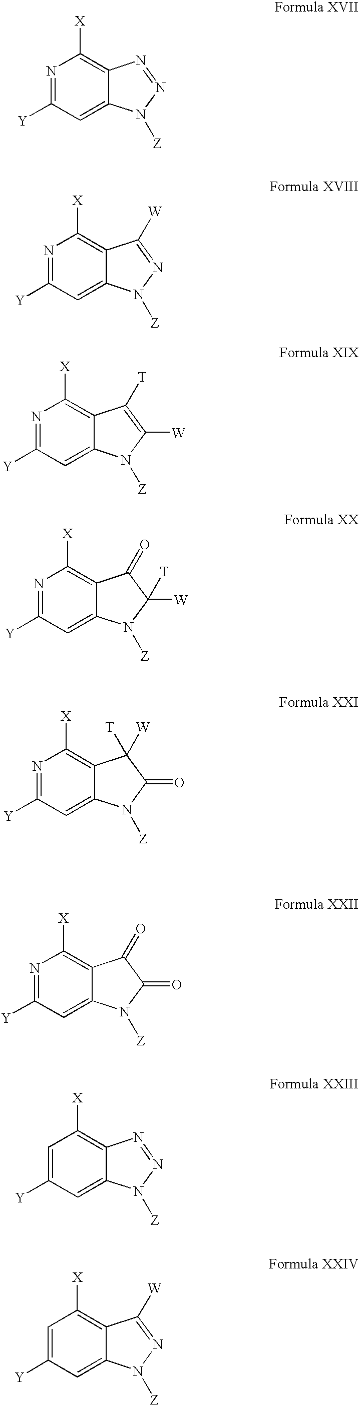 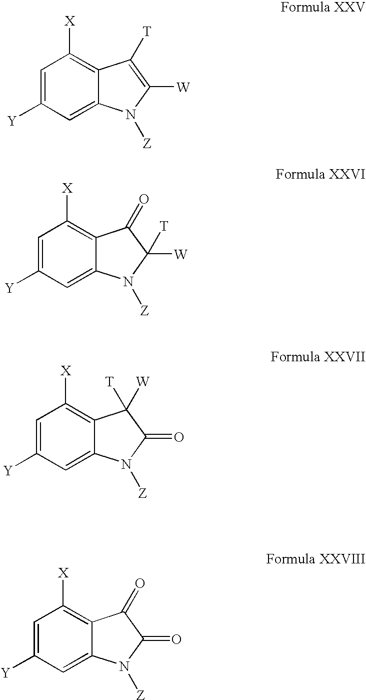
wherein

W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, —NR₁, R₂—OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;

each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and

each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;

each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;

and provided that when X is NH₂ and Y is H, or when Y is NH₂ and X is —OH, W and Z are not both H;

and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

or a pharmaceutically acceptable acid, base, salt, polymorph, solvate, ester, tautomer, stereoisomer or prodrug thereof.

Another aspect of the invention provides a compound of Formula I: 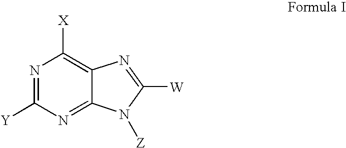
or a pharmaceutically acceptable acid, base, salt, polymorph, solvate, ester, tautomer, stereoisomer or prodrug thereof.

In a more particular embodiment thereof, Z is not ribose (substituted or substituted). In another embodiment thereof, Z is not a heterocyclyl (substituted or substituted).

In a more particular embodiment, X is —NH₂. In another embodiment, Y is F or H.

In another more particular embodiment, W is: 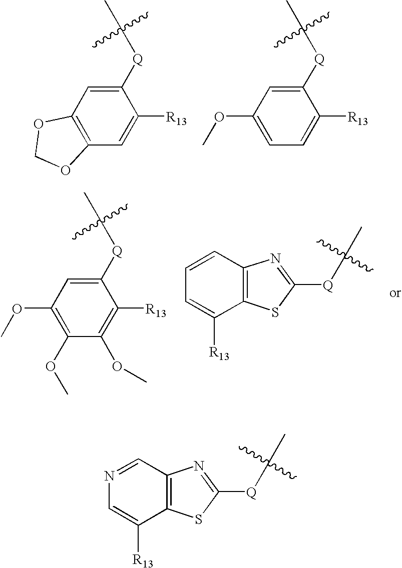
wherein Q is selected from —CR₁R₂—, carbonyl, difluoromethylene, —NR₁—, —O—, —S—, —SO—, and —SO₂—; and
R₁₃ is H, F, Cl, Br, I, OR₁, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C}.

More particularly, W is:

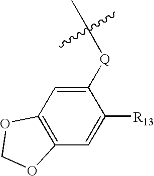

wherein,

R₁₃ is F, Cl, Br or I.

More particularly, Q is CH₂, C═O or CF₂; alternatively Q is O or S. In another embodiment, R₁₃ is I.

In another embodiment, at least one of W, X, Y, or Z is —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C}. More particularly, Z is —R_{A}OSO₂N(R_{C})₂, or —R_{A}N(R_{C})OSO₂R_{C}. More particular still, Z is -heteroalkyl-OSO₂NH₂ or —C₁₋₆ alkyl-OSO₂NH₂.

In another embodiment, Z is —(CH₂)ₚL(CH₂)_{q}OSO₂NH₂;

L is —O—, —S—, N(R_{C}) or triazinyl; and

p and q are independently 1, 2, 3 or 4.

In another embodiment, W is 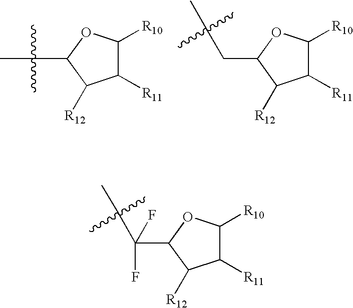
wherein

R₁₀, R₁₁ and R₁₂ are the same or different and each is H, SR_{C}, SOR_{C}, SO₂R₅ OR_{C}, COOR_{C}, CON(R_{C})₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₅R₆, —OSO₂N(R_{C})₂, —N(R_{C}) SO₂OH or —N(R_{C}) SO₂N(R_{C})₂,

and wherein carbons substituted with R₁₁ and R₁₂ may be connected by single or double bond.

Particular compounds of the present invention include:
Sulfamic Acid 2-[6-amino-2-fluoro-8-(6-iodo-benzo[1,3]dioxol-5-ylmethyl)-purin-9-yl]-ethyl ester;
Sulfamic Acid 3-[6-Amino-2-fluoro-8-(6-iodo-benzo[1,3]dioxol-5-ylmethyl)-purin-9-yl]-propyl ester;
Sulfamic Acid 4-[6-amino-2-fluoro-8-(6-iodo-benzo[1,3]dioxol-5-ylmethyl)-purin-9-yl]-butyl ester;
Sulfamic Acid 5-[6-amino-2-fluoro-8-(6-iodo-benzo[1,3]dioxol-5-ylmethyl)-purin-9-yl]-pentyl ester;
Sulfamic Acid 6-[6-amino-2-fluoro-8-(6-iodo-benzo[1,3]dioxol-5-ylmethyl)-purin-9-yl]-hexyl ester;
Sulfamic Acid 7-[6-amino-2-fluoro-8-(6-iodo-benzo[1,3]dioxol-5-ylmethyl)-purin-9-yl]-heptyl ester;
Sulfamic Acid 8-[6-amino-2-fluoro-8-(6-iodo-benzo[1,3]dioxol-5-ylmethyl)-purin-9-yl]-octyl ester;
Sulfamic Acid 2-{2-[6-amino-2-fluoro-8-(6-iodo-benzo[1,3]dioxol-5-ylmethyl)-purin-9-yl]-ethoxy}-ethyl ester;
Sulfamic Acid 3-{2-[6-amino-2-fluoro-8-(6-iodo-benzo[1,3]dioxol-5-ylmethyl)-purin-9-yl]-ethoxy}-propyl ester;
Sulfamic Acid 4-{2-[6-amino-2-fluoro-8-(6-iodo-benzo[1,3]dioxol-5-ylmethyl)-purin-9-yl]-ethoxy}-butyl ester;
Sulfamic Acid 2-({2-[6-amino-2-fluoro-8-(6-iodo-benzo[1,3]dioxol-5-ylmethyl)-purin-9-yl]-ethyl}-isopropyl-amino)-ethyl ester;
Sulfamic Acid 3-({2-[6-amino-2-fluoro-8-(6-iodo-benzo[1,3]dioxol-5-ylmethyl)-purin-9-yl]-ethyl}-isopropyl-amino)-propyl ester;
Sulfamic Acid 4-({2-[6-amino-2-fluoro-8-(6-iodo-benzo[1,3]dioxol-5-ylmethyl)-purin-9-yl]-ethyl}-isopropyl-amino)-butyl ester;
Sulfamic Acid 3-[6-amino-2-fluoro-8-(6-iodo-benzo[1,3]dioxol-5-ylmethyl)-purin-9-yl]-3-methyl-propyl ester;
Sulfamic Acid 4-[6-amino-2-fluoro-8-(6-iodo-benzo[1,3]dioxol-5-ylmethyl)-purin-9-yl]-4-methyl-butyl ester;
Sulfamic Acid 5-[6-amino-2-fluoro-8-(6-iodo-benzo[1,3]dioxol-5-ylmethyl)-purin-9-yl]-5-methyl-pentyl ester;
Sulfamic Acid 2-(4-((6-amino-2-fluoro-8-((6-iodobenzo[d][1,3]dioxol-5-yl)methyl)-9H-purin-9-yl)methyl)-1H-1,2,3-triazol-1-yl)ethyl ester;
Sulfamic Acid 3-[4-({6-amino-2-fluoro-8-[(6-iodo-1,3-benzodioxol-5-yl)methyl]-9H-purin-9-yl}methyl)-1H-1,2,3-triazol-1-yl]propyl ester;
Sulfamic Acid 1-(4-{6-amino-2-fluoro-8-[(6-iodo-1,3-benzodioxol-5-yl)methyl]-9H-purin-9-yl}but-2-yn-1-yl)pyrrolidin-3-ol ester; and
Sulfamic Acid 1-(4-{6-amino-2-fluoro-8-[(6-iodo-1,3-benzodioxol-5-yl)methyl]-9H-purin-9-yl}but-2-yn-1-yl)piperidin-4-ol ester;
   or a pharmaceutically acceptable salt thereof.

Another aspect of the invention provides a composition comprising a compound of Formula I; or a pharmaceutically acceptable acid, base, salt, polymorph, solvate, ester, tautomer, stereoisomer or prodrug thereof; and a pharmaceutically acceptable carrier.

Another aspect of the invention provides a method for inhibiting Hsp90 in a cell, comprising: contacting the cell with a compound of Formula I; or a pharmaceutically acceptable acid, base, salt, polymorph, solvate, ester, tautomer, stereoisomer or prodrug thereof. In a more particular embodiment thereof, the cell exhibits abnormal expression or activity of Hsp90. More particularly, the cell is in vivo.

Another aspect of the invention provides, a method for treating an individual having cancer comprising administering to said individual a compound of Formula I; or a pharmaceutically acceptable acid, base, salt, polymorph, solvate, ester, tautomer, stereoisomer or prodrug thereof; and a pharmaceutically acceptable carrier. In another embodiment, the cancer is selected from the group consisting of breast cancer, small cell lung cancer, amyelocytic leukemia, vulvar cancer, non-small cell lung cancer, colon cancer, colorectal cancer, neuroblastoma, myeloma and prostate cancer. In a more particular embodiment comprises, administering an anti-neoplastic agent to the individual. More particularly, the anti-neoplastic agent is selected from the group of a radioisotope, an antibody, a recombinant protein, traztuzumab, taxol, taxane, gefitinib, imatinib, erlotinib, PTK-787, EKB-569, an alkylating agent, anti-metabolite, epidophyllotoxin, an antineoplastic enzyme, a topoisomerase inhibitor, procarbazine, mitoxantrone, a platinum coordination complex, a growth inhibitor, a hormonal therapeutic agent, an anti-hormonal therapeutic agent, a haematopoietic growth factor, an anthracycline drug, a vinca drug, a mitomycin, a bleomycin, a cytotoxic nucleoside, a tepothilone, a discodermolide, a pteridine drug, a diynesne, a podophyllotoxin, caminomycin, daunorubicin, an aminopterin, methotrexate, methopterin, dichloromethotrexate, mitomycin C, porfiromycin, 5-fluorouracil, 6-mercaptopurine, gemcitabine, cytosine arabinoside, podophyllotoxin, a podo-phyllotoxin, etoposide, etoposide phosphate, teniposide, melphalan, vinblastine, vincristine, leurosidine, vindesine, leurosine, paclitaxel, estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, a pyrodobenzoindole, an interferon and an interleukin.

In other embodiments, the agents disclosed herein find use in combination with each other as well as with other agents, such as where a mixture of one or more of the agents of the present invention are given in combination or where one or more of the agents disclosed herein is given together with some other already known therapeutic agent, possibly as a means of potentiating the affects of such known therapeutic agent or vice versa.

Another embodiment of the present invention provides use of a composition of any one of the previous embodiments for the treatment of an Hsp90 associated condition. More particularly, the present invention provides for use of a compound of any one of the previous embodiments in the manufacture of a medicament for the treatment of an Hsp90 associated condition.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DEFINITIONS

Before describing the present invention in detail, it is to be understood that this invention is not limited to specific compositions or process steps, as such may vary. It should be noted that, as used in this specification and the appended claims, the singular form “a”, “an” and “the” include plural references unless the context clearly dictates otherwise. Thus, for example, reference to “a compound” includes a plurality of compounds.

Unless defined otherwise, all technical and scientific terms used herein have the same meaning as commonly understood by one of ordinary skill in the art to which this invention is related. The following terms are defined for purposes of the invention as described herein.

“Acyl” or “carbonyl” is a radical formed by removal of the hydroxy from a carboxylic acid (i.e., R—C(═O)—). Preferred acyl groups include (for example) acetyl, formyl, and propionyl.

The term “carbon chain” embraces any alkyl, alkenyl, alkynyl, or heteroalkyl, heteroalkenyl, or heteroalkynyl group, which are linear, cyclic, or any combination thereof. If the chain is part of a linker and that linker comprises one or more rings as part of the core backbone, for purposes of calculating chain length, the “chain” only includes those carbon atoms that compose the bottom or top of a given ring and not both, and where the top and bottom of the ring(s) are not equivalent in length, the shorter distance shall be used in determining the chain length. If the chain contains heteroatoms as part of the backbone, those atoms are not calculated as part of the carbon chain length.

“Alkyl” means a saturated hydrocarbon chain having 1 to 15 carbon atoms, preferably 1 to 10, more preferably 1 to 4 carbon atoms. Used alone or in combination, it refers to an optionally substituted straight-chain, or optionally substituted branched-chain saturated hydrocarbon radical having from one to about thirty carbons, more preferably one to twelve carbons. Non-limiting examples of alkyl radicals include methyl, ethyl, n-propyl, isopropyl, n-butyl, isobutyl, sec-butyl, tert-butyl, tert-amyl, pentyl, hexyl, heptyl, octyl and the like.

The term “alkenyl,” or “alkene” alone or in combination, refers to an optionally substituted straight-chain, or optionally substituted branched-chain hydrocarbon radical having one or more carbon-carbon double-bonds and having from two to about thirty carbon atoms, more preferably two to about eighteen carbons. Examples of alkenyl radicals include ethenyl, propenyl, butenyl, 1,3-butadienyl and the like.

The term “alkynyl,” or “alkyne” alone or in combination, refers to an optionally substituted straight-chain or optionally substituted branched-chain hydrocarbon radical having one or more carbon-carbon triple-bonds and having from two to about thirty carbon atoms, more preferably from two to about twelve carbon atoms, from two to about six carbon atoms as well as those having from two to about four carbon atoms. Examples of alkynyl radicals include ethynyl, 2-propynyl, 2-butynyl, 1,3-butadiynyl and the like. A preferred alkynyl group includes 1-(but-2-ynyl)pyrrolidine.

Alkyl, alkene and alkyne chains (referred to collectively as “hydrocarbon chains”) may be straight or branched and may be unsubstituted or substituted. Preferred branched alkyl, alkene and alkyne chains have one or two branches, preferably one branch. Preferred chains are alkyl. Alkyl, alkene and alkyne hydrocarbon chains each may be unsubstituted or substituted with from 1 to 4 substituents; when substituted, preferred chains are mono-, di-, or tri-substituted. Alkyl, alkene and alkyne hydrocarbon chains each may be substituted with halo, hydroxy, aryloxy (e.g., phenoxy), heteroaryloxy, acyloxy (e.g., acetoxy), carboxy, aryl (e.g., phenyl), heteroaryl, cycloalkyl, heteroalkyl, heterocycloalkyl, spirocycle, amino, amido, acylamino, keto, thioketo, cyano, or any combination thereof. Preferred hydrocarbon groups include methyl, ethyl, propyl, isopropyl, butyl, vinyl, allyl, butenyl, and exomethylenyl.

The term “cycloalkyl” embraces cyclic alkyl radicals which include monocyclic, bicyclic, tricyclic, and higher multicyclic alkyl radicals wherein each cyclic moiety has from three to about eight carbon atoms. Examples of cycloalkyl radicals include cyclopropyl, cyclobutyl, cyclopentyl, cyclohexyl and the like. A “lower alkyl” is a shorter alkyl, e.g., one containing from one to about six carbon atoms. The term “cycloalkenyl” refers to cyclic alkenyl radicals which include monocyclic, bicyclic, tricyclic, and higher multicyclic alkenyl radicals wherein each cyclic moiety has from three to about eight carbon atoms. A “lower alkenyl” refers to an alkenyl having from two to about six carbons.

The term “cycloalkynyl” refers to cyclic alkynyl radicals which include monocyclic, bicyclic, tricyclic, and higher multicyclic alkynyl radicals wherein each cyclic moiety has from three to about eight carbon atoms. A “lower alkynyl” refers to an alkynyl having from two to about six carbons.

The terms “heteroalkyl, heteroalkenyl and heteroalkynyl” include optionally substituted alkyl, alkenyl and alkynyl structures, as described above, and which have one or more skeletal chain atoms selected from an atom other than carbon, e.g., oxygen, nitrogen, sulfur, phosphorous or combinations thereof.

Also, as referred to herein, a “lower” alkyl, alkene or alkyne moiety (e.g., “lower alkyl”) is a chain comprised of 1 to 10, preferably from 1 to 8, carbon atoms in the case of alkyl and 2 to 10, preferably 2 to 8, carbon atoms in the case of alkene and alkyne.

“Alkoxy” or “alkoxyl” means an oxygen radical having a hydrocarbon chain substituent, where the hydrocarbon chain is an alkyl or alkenyl (i.e., —O-alkyl or —O— alkenyl). Examples of alkoxy radicals include methoxy, ethoxy, n-propoxy, isopropoxy, n-butoxy, iso-butoxy, sec-butoxy, tert-butoxy, allyloxy and the like.

“Aryl” is an aromatic hydrocarbon ring. Aryl rings are monocyclic or fused bicyclic ring systems. Monocyclic aryl rings contain 6 carbon atoms in the ring. Monocyclic aryl rings are also referred to as phenyl rings. Bicyclic aryl rings contain from 8 to 17 carbon atoms, preferably 9 to 12 carbon atoms, in the ring. Bicyclic aryl rings include ring systems wherein one ring is aryl and the other ring is aryl, cycloalkyl, or heterocycloakyl. Preferred bicyclic aryl rings comprise 5-, 6- or 7-membered rings fused to 5-, 6-, or 7-membered rings. Aryl rings may be unsubstituted or substituted with from 1 to 4 substituents on the ring. Aryl may be substituted with halo, cyano, nitro, hydroxy, carboxy, amino, acylamino, alkyl, heteroalkyl, haloalkyl, phenyl, aryloxy, alkoxy, heteroalkyloxy, carbamyl, haloalkyl, methylenedioxy, heteroaryloxy, or any combination thereof. Preferred aryl rings include naphthyl, tolyl, xylyl, and phenyl. The most preferred aryl ring radical is phenyl.

“Aryloxy” is an oxygen radical having an aryl substituent (i.e., —O-aryl). Preferred aryloxy groups include (for example) phenoxy, napthyloxy, methoxyphenoxy, and methylenedioxyphenoxy.

“Cycloalkyl” is a saturated or unsaturated hydrocarbon ring. Cycloalkyl rings are not aromatic. Cycloalkyl rings are monocyclic, or are fused, spiro, or bridged bicyclic ring systems. Monocyclic cycloalkyl rings contain from about 3 to about 9 carbon atoms, preferably from 3 to 7 carbon atoms, in the ring. Bicyclic cycloalkyl rings contain from 7 to 17 carbon atoms, preferably from 7 to 12 carbon atoms, in the ring. Preferred bicyclic cycloalkyl rings comprise 4-, 5-6- or 7-membered rings fused to 5-, 6-, or 7-membered rings. Cycloalkyl rings may be unsubstituted or substituted with from 1 to 4 substituents on the ring. Cycloalkyl may be substituted with halo, cyano, alkyl, heteroalkyl, haloalkyl, phenyl, keto, hydroxy, carboxy, amino, acylamino, aryloxy, heteroaryloxy, or any combination thereof. Preferred cycloalkyl rings include cyclopropyl, cyclopentyl, and cyclohexyl.

“Halo” or “halogen” is fluoro, chloro, bromo or iodo. Preferred halo are fluoro, chloro and bromo; more preferred typically are chloro and fluoro, especially fluoro.

“Haloalkyl” is a straight, branched, or cyclic hydrocarbon substituted with one or more halo substituents. Preferred are C₁-C₁₂ haloalkyls; more preferred are C₁-C₆ haloalkyls; still more preferred still are C₁-C₃ haloalkyls. Preferred halo substituents are fluoro and chloro. The most preferred haloalkyl is trifluoromethyl.

“Heteroatom” is a nitrogen, sulfur, or oxygen atom. Groups containing more than one heteroatom may contain different heteroatoms.

“Heteroalkyl” is a saturated or unsaturated chain containing carbon and at least one heteroatom, wherein no two heteroatoms are adjacent. Heteroalkyl chains contain from 2 to 15 member atoms (carbon and heteroatoms) in the chain, preferably 2 to 10, more preferably 2 to 5. For example, alkoxy (i.e., —O-alkyl or —O-heteroalkyl) radicals are included in heteroalkyl. Heteroalkyl chains may be straight or branched. Preferred branched heteroalkyl have one or two branches, preferably one branch. Preferred heteroalkyl are saturated. Unsaturated heteroalkyl have one or more carbon-carbon double bonds and/or one or more carbon-carbon triple bonds. Preferred unsaturated heteroalkyls have one or two double bonds or one triple bond, more preferably one double bond. Heteroalkyl chains may be unsubstituted or substituted with from 1 to 4 substituents. Preferred substituted heteroalkyl are mono-, di-, or tri-substituted. Heteroalkyl may be substituted with lower alkyl, haloalkyl, halo, hydroxy, aryloxy, heteroaryloxy, acyloxy, carboxy, monocyclic aryl, heteroaryl, cycloalkyl, heteroalkyl, heterocycloalkyl, spirocycle, amino, acylamino, amido, keto, thioketo, cyano, or any combination thereof. Where a group is described, for example, as an alkyl derivative, such as “-ethylpyridine” the dash “-” indicate point of attachment of the substituent. Thus, “-ethylpyridine” means attachment of ethylpyridine via the ethyl portion of the group whereas “ethylpyridine-” means attachment via the pyridinyl ring.

“Heteroaryl” is an aromatic ring containing carbon atoms and from 1 to about 6 heteroatoms in the ring. Heteroaryl rings are monocyclic or fused bicyclic ring systems. Monocyclic heteroaryl rings contain from about 5 to about 9 member atoms (carbon and heteroatoms), preferably 5 or 6 member atoms, in the ring. Bicyclic heteroaryl rings contain from 8 to 17 member atoms, preferably 8 to 12 member atoms, in the ring. Bicyclic heteroaryl rings include ring systems wherein one ring is heteroaryl and the other ring is aryl, heteroaryl, cycloalkyl, or heteroalkyl, heterocycloalkyl. Preferred bicyclic heteroaryl ring systems comprise 5-, 6- or 7-membered rings fused to 5-, 6-, or 7-membered rings. Heteroaryl rings may be unsubstituted or substituted with from 1 to 4 substituents on the ring. Heteroaryl may be substituted with halo, cyano, nitro, hydroxy, carboxy, amino, acylamino, alkyl, heteroalkyl, haloalkyl, phenyl, alkoxy, aryloxy, heteroaryloxy, or any combination thereof. Preferred heteroaryl rings include, but are not limited to, the following:

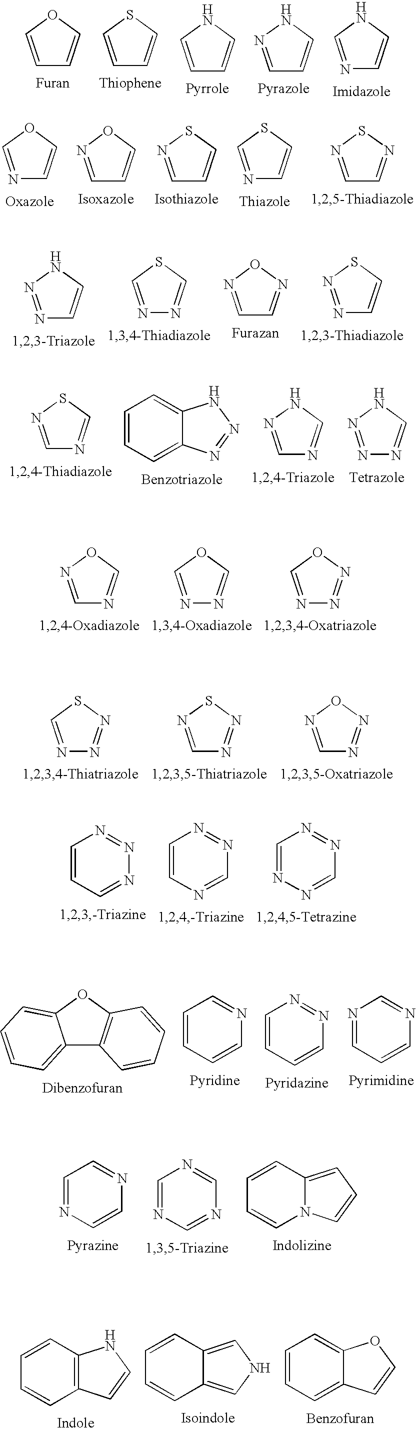

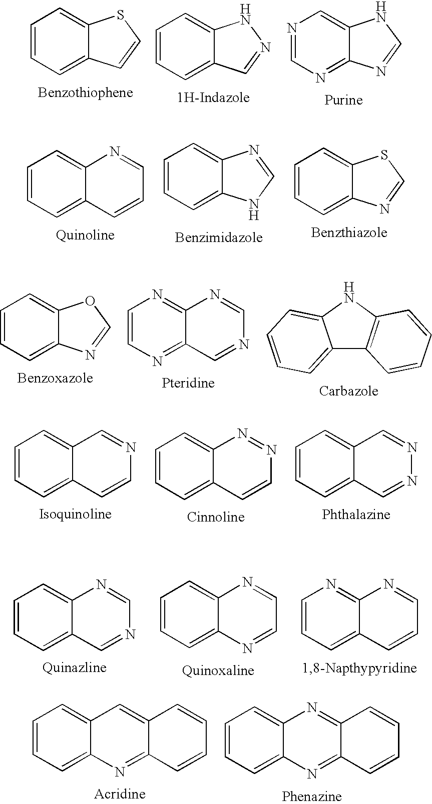

A fused heteroaryl radical may contain from two to four fused rings and where the ring of attachment is a heteroaromatic ring, the other individual rings within the fused ring system may be aromatic, heteroaromatic, alicyclic or heterocyclic. The term heteroaryl also includes mono-heteroaryls or fused heteroaryls having from five to about twelve skeletal ring atoms, as well as those having from five to about ten skeletal ring atoms. The term “lower heteroaryl” refers to a heteroaryl having five to about ten skeletal ring atoms, e.g., pyridyl, thienyl, pyrimidyl, pyrazinyl, pyrrolyl, or furanyl.

“Heteroaryloxy” is an oxygen radical having a heteroaryl substituent (i.e., —O— heteroaryl). Preferred heteroaryloxy groups include (for example) pyridyloxy, furanyloxy, (thiophene)oxy, (oxazole)oxy, (thiazole)oxy, (isoxazole)oxy, pyrmidinyloxy, pyrazinyloxy, and benzothiazolyloxy.

“Heterocycloalkyl” is a saturated or unsaturated ring containing carbon atoms and from 1 to about 4 (preferably 1 to 3) heteroatoms in the ring. Heterocycloalkyl rings are not aromatic. Heterocycloalkyl rings are monocyclic, or are fused, bridged, or spiro bicyclic ring systems. Monocyclic heterocycloalkyl rings contain from about 3 to about 9 member atoms (carbon and heteroatoms), preferably from 5 to 7 member atoms, in the ring. Bicyclic heterocycloalkyl rings contain from 7 to 17 member atoms, preferably 7 to 12 member atoms, in the ring. Bicyclic heterocycloalkyl rings contain from about 7 to about 17 ring atoms, preferably from 7 to 12 ring atoms. Bicyclic heterocycloalkyl rings may be fused, spiro, or bridged ring systems. Preferred bicyclic heterocycloalkyl rings comprise 5-, 6- or 7-membered rings fused to 5-, 6-, or 7-membered rings. Heterocycloalkyl rings may be unsubstituted or substituted with from 1 to 4 substituents on the ring. Heterocycloalkyl may be substituted with halo, cyano, hydroxy, carboxy, keto, thioketo, amino, acylamino, acyl, amido, alkyl, heteroalkyl, haloalkyl, phenyl, alkoxy, aryloxy or any combination thereof. Preferred substituents on heterocycloalkyl include halo and haloalkyl. Preferred heterocycloalkyl rings include, but are not limited to, the following:

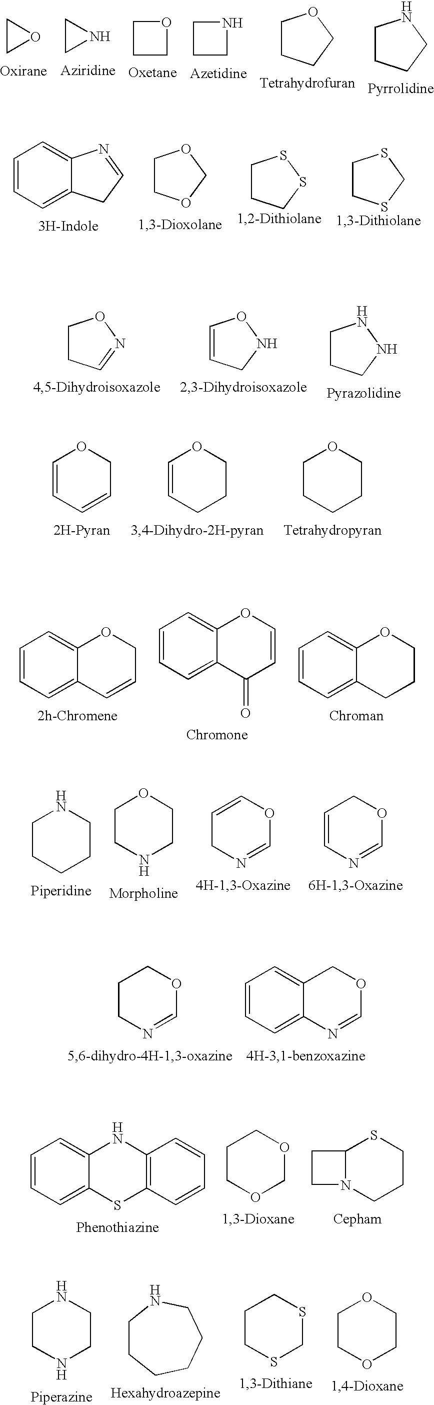

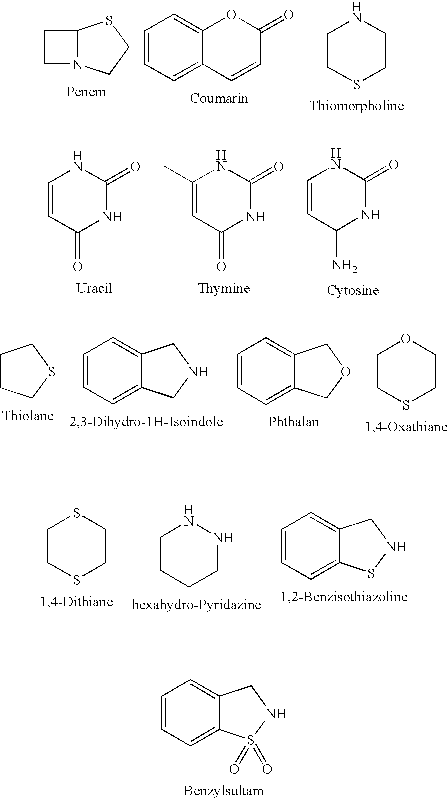

While alkyl, heteroalkyl, cycloalkyl, and heterocycloalkyl groups may be substituted with hydroxy, amino, and amido groups as stated above, the following are not envisioned in the invention:

Enols (OH attached to a carbon bearing a double bond).

Amino groups attached to a carbon bearing a double bond (except for vinylogous amides).

More than one hydroxy, amino, or amido attached to a single carbon (except where two nitrogen atoms are attached to a single carbon atom and all three atoms are member atoms within a heterocycloalkyl ring).

Hydroxy, amino, or amido attached to a carbon that also has a heteroatom attached to it.

A “pharmaceutically-acceptable salt” is a cationic salt formed at any acidic (e.g., carboxylic acid) group, or an anionic salt formed at any basic (e.g., amino) group. Many such salts are known in the art, as described in World Patent Publication 87/05297, Johnston et al., published Sep. 11, 1987 incorporated by reference herein. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-napthalenesulfonate, nicotinate, nitrate, oxalate; palmoate, pectinate, persulfate, 3-phenylpropionate, phosphate, picrate, pivalate, propionate, salicylate, succinate, sulfate, tartrate, thiocyanate, tosylate and undeconate. Other acids, such as oxalic, while not in themselves pharmaceutically acceptable, may be employed in the preparation of salts useful as intermediates in obtaining the compounds of the invention and their pharmaceutically acceptable acid addition salts. Preferred cationic salts include the alkali metal salts (such as sodium and potassium), and alkaline earth metal salts (such as magnesium and calcium) and organic salts. Preferred anionic salts include the halides (such as chloride salts), sulfonates, carboxylates, phosphates, and the like.

Compounds of the present invention that contain one or more acidic functional groups are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of some of the bases that can be used include sodium hydroxide, potassium hydroxide, choline hydroxide, sodium carbonate, N⁺(C₁₋₄ alkyl)₄, and the like. Representative organic amines useful for the formation of base addition salts include ethylamine, diethylamine, ethylenediamine, ethanolamine, diethanolamine, piperazine and the like. This invention also envisions the quaternization of any basic nitrogen-containing groups of the compounds disclosed herein. Water or oil-soluble or dispersible products may be obtained by such quaternization.

Such salts are well understood by the skilled artisan, and the skilled artisan is able to prepare any number of salts given the knowledge in the art. Furthermore, it is recognized that the skilled artisan may prefer one salt over another for reasons of solubility, stability, formulation ease and the like. Determination and optimization of such salts is within the purview of the skilled artisan's practice.

A “solvate” is a complex formed by the combination of a solute (e.g., a metalloprotease inhibitor) and a solvent (e.g., water). See J. Honig et al., The Van Nostrand Chemist's Dictionary, p. 650 (1953). Pharmaceutically-acceptable solvents used according to this invention include those that do not substantially disrupt non-HSP90 biological function and include, for example, hydrates and others known or readily determined by the skilled artisan).

The terms “optical isomer”, “stereoisomer”, and “diastereomer” have the accepted meanings (see, e.g., Hawley's Condensed Chemical Dictionary, 11th Ed.). The illustration of specific protected forms and other derivatives of the compounds of the instant invention is not intended to be limiting. The application of other useful protecting groups, salt forms, prodrugs etc. is within the ability of the skilled artisan.

The term “membered ring” can embrace any cyclic structure, including aromatic, heteroaromatic, alicyclic, heterocyclic and polycyclic fused ring systems as described below. The term “membered” is meant to denote the number of skeletal atoms that constitute the ring. Thus, for example, pyridine, pyran, and pyrimidine are six-membered rings and pyrrole, tetrahydrofuran, and thiophene are five-membered rings.

The term “aryl,” alone or in combination, refers to an optionally substituted aromatic hydrocarbon radical of six to about twenty ring atoms, and includes mono-aromatic rings and fused aromatic ring. A fused aromatic ring radical contains from two to four fused rings where the ring of attachment is an aromatic ring, and the other individual rings within the fused ring may be aromatic, heteroaromatic, alicyclic or heterocyclic. Further, the term aryl includes mono-aromatic ring and fused aromatic rings containing from six to about twelve carbon atoms, as well as those containing from six to about ten carbon atoms. Examples of aryl groups include, without limitation, phenyl, naphthyl, anthryl, chrysenyl, and benzopyrenyl ring systems. The term “lower aryl” refers to an aryl having six to about ten skeletal ring carbons, e.g., phenyl and naphthyl ring systems.

The term “heterocyclic” refers to optionally substituted saturated or unsaturated nonaromatic ring radicals containing from five to about twenty ring atoms where one or more of the ring atoms are heteroatoms such as, for example, oxygen, nitrogen, sulfur, and phosphorus. The term alicyclic includes mono-heterocyclic and fused heterocyclic ring radicals. A fused heterocyclic radical may contain from two to four fused rings where the attaching ring is a heterocyclic, and the other individual rings within the fused heterocyclic radical may be aromatic, heteroaromatic, alicyclic or heterocyclic. The term heterocyclic also includes mono-heterocyclic and fused alicyclic radicals having from five to about twelve skeletal ring atoms, as well as those having from five to about ten skeletal ring atoms. Example of heterocyclics include without limitation, tetrahydrofuranyl, benzodiazepinyl, tetrahydroindazolyl, dihyroquinolinyl, and the like. The term “lower heterocyclic” refers to a heterocyclic ring system having five to about ten skeletal ring atoms, e.g., dihydropyranyl, pyrrolidinyl, indolyl, piperidinyl, piperazinyl, and the like.

The term “alkylaryl,” alone or in combination, refers to an aryl radical as defined above in which one H atom is replaced by an alkyl radical as defined above, such as, for example, tolyl, xylyl and the like.

The term “arylalkyl,” or “ara-alkyl,” alone or in combination, refers to an alkyl radical as defined above in which one H atom is replaced by an aryl radical as defined above, such as, for example, benzyl, 2-phenylethyl and the like.

The term “heteroarylalkyl” refers to an alkyl radical as defined above in which one H atom is replaced by a heteroaryl radical as defined above, each of which may be optionally substituted but wherein the aryl group is attached to a larger core structure with the alkyl group being the terminal moiety.

The term “alkylheteroaryl” refers to an alkyl radical as defined above in which one H atom is replaced by a heteroaryl radical as defined above, each of which may be optionally substituted but wherein the alkyl group is attached to a larger core structure with the heteroaryl group being the terminal moiety.

The term “aryloxy,” alone or in combination, refers to an aryl ether radical wherein the term aryl is defined as above. Examples of aryloxy radicals include phenoxy, benzyloxy and the like.

The term “alkylthio,” alone or in combination, refers to an alkyl thio radical, alkyl-S—, wherein the term alkyl is as defined above.

The term “arylthio,” alone or in combination, refers to an aryl thio radical, aryl-S—, wherein the term aryl is as defined above.

The term “heteroarylthio” refers to the group heteroaryl-S—, wherein the term heteroaryl is as defined above.

The term “acyl” refers to a radical —C(O)R where R includes alkyl, alkenyl, alkynyl, aryl, heteroaryl, alicyclic, heterocyclic, arylalkyl or heteroarylalkyl wherein the alkyl, alkenyl, alkynyl, aryl, heteroaryl, alicyclic, heterocyclic, arylalkyl or heteroaryl alkyl groups may be optionally substituted.

The term “acyloxy” refers to the ester group —OC(O)R, where R is H, alkyl, alkenyl, alkynyl, aryl, heteroaryl, alicyclic, heterocyclic, arylalkyl, or heteroarylalkyl wherein the alkyl, alkenyl, alkynyl, aryl, heteroaryl, alicyclic, heterocyclic, arylalkyl or heteroarylalkyl may be optionally substituted.

The term “carboxy esters” refers to —C(O)OR where R is alkyl, aryl or arylalkyl, wherein the alkyl, aryl and arylalkyl groups may be optionally substituted.

The term “carboxamido” refers to the structure_—C(O)NRR′ where nitrogen is attached to the carbonyl carbon and each of R and R′ are independently selected from the group consisting of H, alkyl, aryl, heteroaryl, alicyclic, heterocyclic, arylalkyl and heteroarylalkyl, wherein the alkyl, aryl, heteroaryl, alicyclic, heterocyclic, or arylalkyl groups may be optionally substituted.

The term “oxo” refers to double-bonded oxygen, depicted as ═O.

The term “halogen” includes F, Cl, Br and I.

The terms “haloalkyl, haloalkenyl, haloalkynyl and haloalkoxy” include alkyl, alkenyl, alkynyl and alkoxy structures, as described above, that are substituted with one or more fluorines, chlorines, bromines or iodines, or with combinations thereof.

The terms “perhaloalkyl, perhaloalkyloxy and perhaloacyl” refer to alkyl, alkyloxy and acyl radicals as described above, that all the H atoms are substituted with fluorines, chlorines, bromines or iodines, or combinations thereof.

The terms “cycloalkyl, arylalkyl, aryl, heteroaryl, alicyclic, heterocyclic, alkyl, alkynyl, alkenyl, haloalkyl, and heteroalkyl” include optionally substituted cycloalkyl, arylalkyl, aryl, heteroaryl, alicyclic, heterocyclic, alkyl, alkynyl, alkenyl, haloalkyl and heteroalkyl groups.

The terms “alkylamino”, refers to the group —NHR′ where R is independently selected from alkyl.

The terms “dialkylamino”, refers to the group —NRR′ where R and R′ are alkyls.

The term “sulfide” refers to a sulfur atom covalently linked to two atoms; the formal oxidation state of said sulfur is (II). The term “thioether” may be used interchangeably with the term “sulfide.”

The term “sulfoxide” refers to a sulfur atom covalently linked to three atoms, at least one of which is an oxygen atom; the formal oxidation state of said sulfur atom is (IV).

The term “sulfone” refers to a sulfur atom covalently linked to four atoms, at least two of which are oxygen atoms; the formal oxidation state of said sulfur atom is (VI).

The term “ribose” refers to substituted and unsubstituted moiety having the structure: 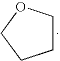
“Z is not ribose” indicates the Z substituent is not a substituted or unsubstituted ribose group directly bound to the parent molecule.

The terms “optional” or “optionally” mean that the subsequently described event or circumstance may but need not occur, and that the description includes instances where the event or circumstance occurs and instances in which it does not. For example, “aryl optionally mono- or di-substituted with an alkyl” means that the alkyl may but need not be present, or either one alkyl or two may be present, and the description includes situations where the aryl is substituted with one or two alkyls and situations where the aryl is not substituted with an alkyl.

“Optionally substituted” groups may be substituted or unsubstituted. The substituents of an “optionally substituted” group may include, without limitation, one or more substituents independently selected from the following groups or designated subsets thereof: C₁₋₆ alkyl, C₂₋₆ alkenyl, C₂₋₆ alkynyl, lower aryl, heteroaryl, alicyclic, heterocyclic, arylalkyl, heteroarylalkyl, lower alkoxy, lower aryloxy, amino, alkylamino, dialkylamino, diarylalkylamino, alkylthio, arylthio, heteroarylthio, oxo, oxa, carbonyl (—C(O)), carboxyesters (—C(O)OR), carboxamido (—C(O)NH₂), carboxy, acyloxy, —H, halo, —CN, —NO₂, —N₃, —SH, —OH, —C(O)CH₃, perhaloalkyl, perhaloalkoxy, perhaloacyl, guanidine, pyridinyl, thiophene, furanyl, indole, indazole, esters, amides, phosphonates, phosphonic acid, phosphates, phosphoramides, sulfonates, sulfones, sulfates, sulphonamides, carbamates, ureas, thioureas and thioamides, thioalkyls. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

The term “pyridine-1-oxy” also means “pyridine-N-oxy.”

Some of the compounds of the present invention may contain one or more chiral centers and therefore may exist in enantiomeric and diastereomeric forms. The scope of the present invention is intended to cover all isomers per se, as well as mixtures of cis and trans isomers, mixtures of diastereomers and racemic mixtures of enantiomers (optical isomers) as well. Further, it is possible using well known techniques to separate the various forms, and some embodiments of the invention may feature purified or enriched species of a given enantiomer or diastereomer.

It will become apparent from the position of the substituent in a chain whether it is monovalent, divalent, etc. For example: —R_{A}OR_{B}, R_{A} is divalent. Therefore, if R_{A} is alkynyl, the alkynyl group will be divalent and optionally substituted, wherein the substituted group can be part of the divalent linkage or an appendage of the alkynyl group.

“Treating” or “treatment” of a disease refers to 1) preventing the disease from occurring in a subject that is predisposed or does not yet display symptoms of the disease; 2) inhibiting the disease or arresting its development; or 3) ameliorating or causing regression of the disease.

A “pharmacological composition” refers to a mixture of one or more of the compounds described herein, or pharmaceutically acceptable salts thereof, with other chemical components, such as pharmaceutically acceptable carriers and/or excipients. The purpose of a pharmacological composition is to facilitate administration of a compound to an organism.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. A physiologically acceptable carrier should not cause significant irritation to an organism and does not abrogate the biological activity and properties of the administered compound.

An “excipient” refers to an inert substance added to a pharmacological composition to further facilitate administration of a compound. Examples of excipients include but are not limited to calcium carbonate, calcium phosphate, various sugars and types of starch, cellulose derivatives, gelatin, vegetable oils and polyethylene glycols.

A “pharmaceutically effective amount” means an amount which is capable of providing a therapeutic and/or prophylactic effect. The specific dose of compound administered according to this invention to obtain therapeutic and/or prophylactic effect will, of course, be determined by the particular circumstances surrounding the case, including, for example, the specific compound administered, the route of administration, the condition being treated, and the individual being treated. A typical daily dose (administered in single or divided doses) will contain a dosage level of from about 0.01 mg/kg to about 50-100 mg/kg of body weight of an active compound of the invention. Preferred daily doses generally will be from about 0.05 mg/kg to about 20 mg/kg and ideally from about 0.1 mg/kg to about 10 mg/kg. Factors such as clearance rate, half-life and maximum tolerated dose (MTD) have yet to be determined but one of ordinary skill in the art can determine these using standard procedures.

In some method embodiments, the preferred therapeutic effect is the inhibition, to some extent, of the growth of cells characteristic of a proliferative disorder, e.g., breast cancer. A therapeutic effect will also normally, but need not, relieve to some extent one or more of the symptoms other than cell growth or size of cell mass. A therapeutic effect may include, for example, one or more of 1) a reduction in the number of cells; 2) a reduction in cell size; 3) inhibition (i.e., slowing to some extent, preferably stopping) of cell infiltration into peripheral organs, e.g., in the instance of cancer metastasis; 3) inhibition (i.e., slowing to some extent, preferably stopping) of tumor metastasis; 4) inhibition, to some extent, of cell growth; and/or 5) relieving to some extent one or more of the symptoms associated with the disorder.

As used herein, the term “IC₅₀” refers to an amount, concentration or dosage of a particular test compound that achieves a 50% inhibition of a maximal response in an assay that measures such response. In some method embodiments of the invention, the “IC₅₀” value of a compound of the invention can be greater for normal cells than for cells exhibiting a proliferative disorder, e.g., breast cancer cells. The value depends on the assay used.

By a “standard” is meant a positive or negative control. A negative control in the context of HER2 expression levels is, e.g., a sample possessing an amount of HER2 protein that correlates with a normal cell. A negative control may also include a sample that contains no HER2 protein. By contrast, a positive control does contain HER2 protein, preferably of an amount that correlates with overexpression as found in proliferative disorders, e.g., breast cancers. The controls may be from cell or tissue samples, or else contain purified ligand (or absent ligand), immobilized or otherwise. In some embodiments, one or more of the controls may be in the form of a diagnostic “dipstick.”

By “selectively targeting” is meant affecting one type of cell to a greater extent than another, e.g., in the case of cells with high as opposed to relatively low or normal HER2 levels.

### DETAILED DESCRIPTION OF THE INVENTION

Compounds of the invention and their polymorphs, solvates, esters, tautomers, diastereomers, enantiomers, pharmaceutically acceptable salts or prodrugs show utility for inhibiting HSP90 and treating and/or preventing diseases that are HSP90-dependent.

The compound 2-chloro-5′-sulfamoyladenosine (CSA) has been shown to be an antibiotic but has been found toxic to mice and to inhibit protein synthesis (see, for example, Takahashi and Bepu, J. Antibiotics, 35, 939-947 (1982)) It was already known that 5′-sulfamoyladenosine inhibited protein synthesis. (See Bloch and Coutsogeogopolous, Biochemistry, 10, 4394-4398 (1971). For these reasons, it has not proven to be a useful drug.

In accordance with the present invention, it has been discovered that CSA also inhibits HSP90. This compound is notable for its sulfamoyl group on the 5′-position of the ribosyl moiety of the adenosine molecule and the presence of a chlorine atom at the 2-position of adenine.

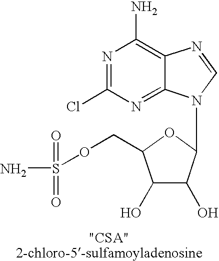

The discovery of the naturally occurring HSP90 inhibitor 2-chloro-5′-sulfamoyladenosine and the initial structure activity relationship shown below ultimately lead to the HSP90 inhibitors of the present invention.

| | | HSP90 |
|---|---|---|
| | | CE |
| | | IC50 |
| Sample description | Molecular Structure | (uM) |
| Chloroadenosine (CA) | 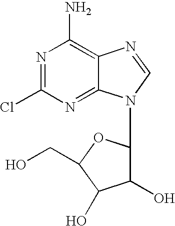 | 50 |
| CSA | 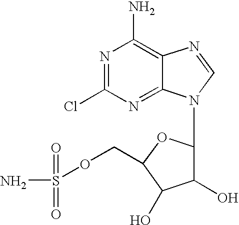 | 1 |
| Fluoroadenosine (FA) | 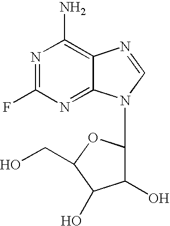 | 22 |
| FSA | 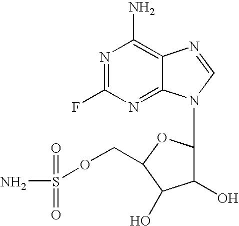 | 0.7 |
| Adenosine (A) | 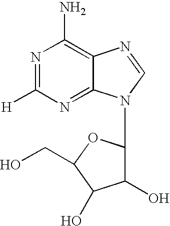 | >188 |
| SA | 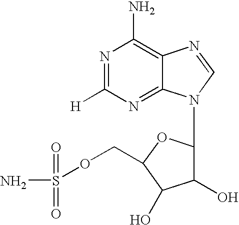 | 40 |
| AMP | 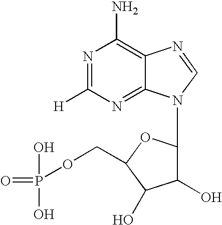 | >144 |

In one embodiment, the heterocyclic ring system based compounds of the present invention are for example adenine derivatives possessing halogen substituents on the adenine ring and containing a sulfamoyl moiety at advantageous positions within the molecule.

In one embodiment, the present invention relates to a compound having the of general Formula A 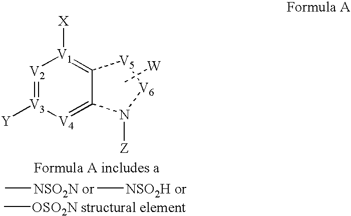
wherein each V is independently C or N, wherein if V₂ or V₄ is C said C is substituted only with hydrogen, and wherein each of V₅ and V₆ is unsubstituted or is independently substituted with one or more substitutents independently selected from W,
and wherein

W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, —NR₁R₂—OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;

each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and

each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;

each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;

R₃ and R₄ are the same or different and each is H, ═O, SR₅, SOR₅, SO₂R₅ OR₅, COOR₅, CONR₅R₆, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₅R₆, —OSO₂NR₅R₆, —N(R₁) SO₂OH or —N(R₈) SO₂NR₅R₆;

R₅ and R₆ are the same or different and each is (CR₇R₈)ₙ-DSO₂NR₃R₄ (wherein D is O or N, and wherein if X is N said N has attached an R₈ and wherein n=0, 1, 2, 3, 4, 5, 6, 7 or 8), C₂₋₆ alkenyl, C₂₋₆ alkynyl, —R_{A}OR_{B}—, —R_{A}NR_{B}, —R_{A}NR_{B} or —R_{A}SR_{B}, —R_{A}SOR_{B} or —R_{A}SO₂R_{B};

R₇ is H, ═O, SR₆, SOR₆, SO₂R₆, OR₆, —CN, C₁₋₆ alkyl, C₂₋₆ alkenyl, C₂₋₆ alkynyl, —YOZ-, —YNZ, —YNR₁Z, —YSZ, —YSOZ or —YSO₂Z cycloalkyl, heteroalkyl, heterocycloalkyl, aryl, heteroaryl, alkylaryl, arylalkyl, alkylheteroaryl, heteroarylalkyl, NR₅R₆, —N(R₈)SO₂NR₅R₆, —N(R₁)SO₂OH or —OSO₂NR₅R₆;

R₈ is H, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, cycloalkyl, heteroalkyl, heterocycloalkyl, aryl, heteroaryl, alkylaryl, arylalkyl, alkylheteroaryl, heteroarylalkyl, NR₅R₆, —N(R₉)SO₂NR₅R₆—N(R₁) SO₂OH or —OSO₂NR₅R₆; and

R₉ is H, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₅R₆, —N(R₁)SO₂OH or —OSO₂NR₅R₆;

and wherein any of said C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl may contain at least one substituent selected from H, F, Cl, Br, I, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, NR₅R₆, —N(R₈)SO₂NR₅R₆, —N(R₈)SO₂OH or —OSO₂NR₅R₆, and wherein when more than one said substituent is present said substituents may be fused to form one or more additional ring systems;

and provided that when X is NH₂ and Y is H, or when Y is NH₂ and X is —OH, W and Z are not both H;

and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};

including any pharmaceutically acceptable acid, base, salt, polymorph, solvate, ester, tautomer, enantiomer, diastereomer or prodrug thereof.

In specific examples of such compounds, X is —NH₂, or Y is F or H.

In particular examples, wherein W is selected from: 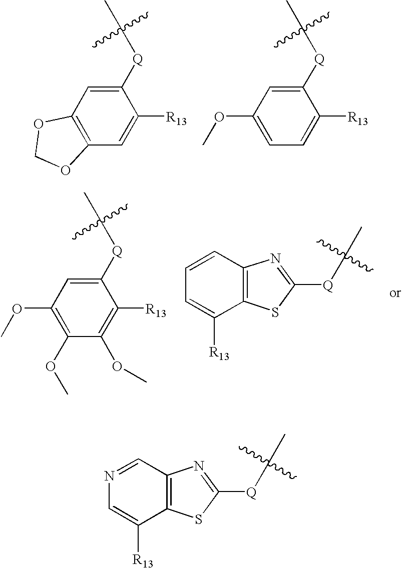
wherein Q is selected from —CR₁R₂—, carbonyl, difluoromethylene, —NR₁—, —O—, —S—, —SO—, and —SO₂—; and
R₁₃ is H, F, Cl, Br, I, OR₁, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C}.

In one specific embodiment the compound has the structure of Formula I 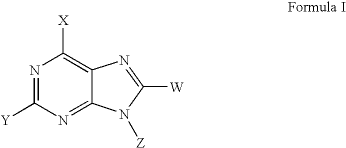
wherein X is —NH₂, Y is F and W is 6-iodo-benzo[1,3]dioxol-5-yl-methyl.

In a specific example of a compound of Formula I, W is heteroalkyl, heterocycloalkyl, for example, having the structure 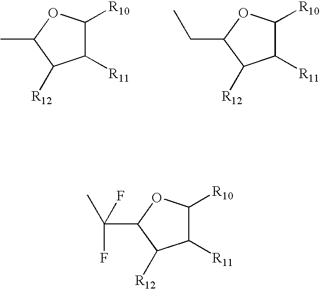
wherein

R₁₀, R₁₁ and R₁₂ are the same or different and each is H, SR_{C}, SOR_{C}, SO₂R₅ OR_{C}, COOR_{C}, CON(R_{C})₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₅R₆, —OSO₂N(R_{C})₂, —N(R_{C}) SO₂OH or —N(R_{C}) SO₂N(R_{C})₂,

and wherein carbons substituted with R₁₁ and R₁₂ may be connected by single or double bond,

In specific examples of the compounds of the invention, such compounds are those having the structure of Formula A,
wherein Z has the structural element —NSO₂N or —NSO₂OH or OSO₂N in addition or as part C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SORB or —R_{A}SO₂R_{B} or in addition or as part of a structure selected from

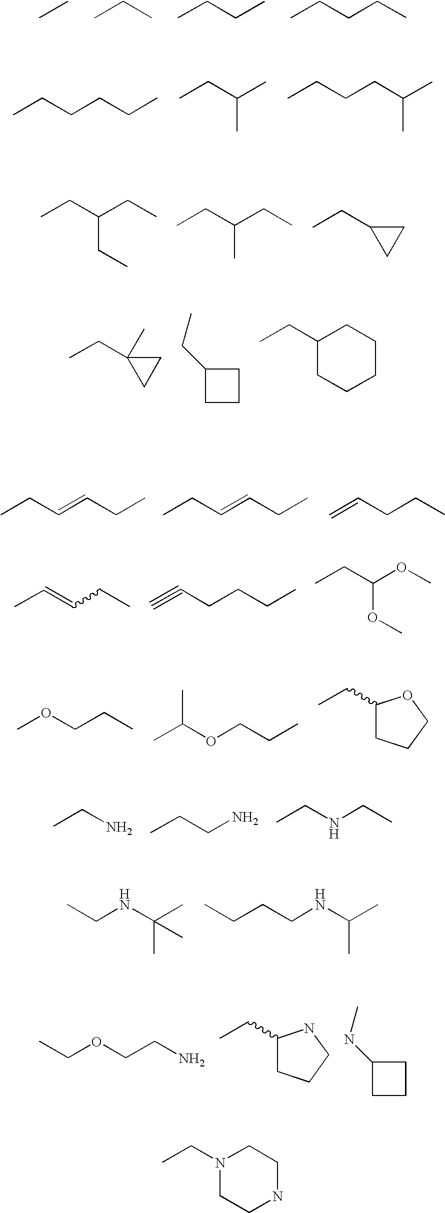

In specific embodiments of the foregoing, X is NH₂ and/or Y is F. In other such embodiments, W is 6-iodo-benzo[1,3]dioxol-5-yl-methyl.

In other specific examples of the compounds of the invention, such compounds are those having Formulas II, III and IV, as follows and wherein all substituents are as defined elsewhere herein:

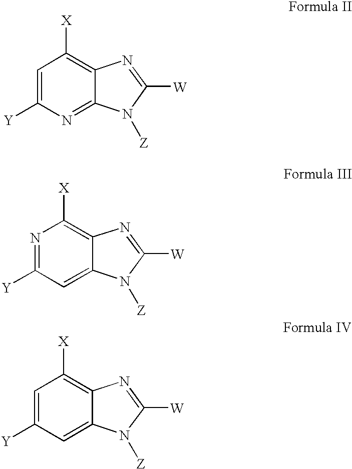

In other specific examples of the compounds of the invention, such compounds are those having Formulas V through XXVII, as follows and wherein all substituents are as defined elsewhere herein: 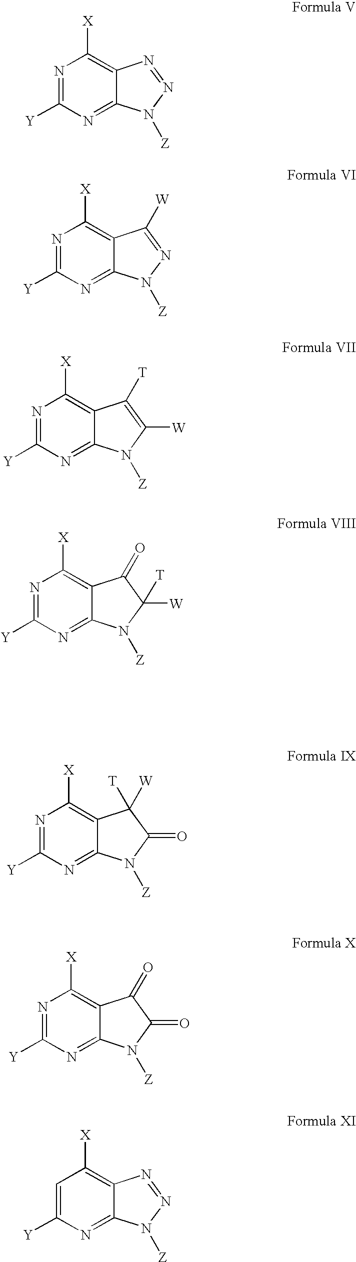 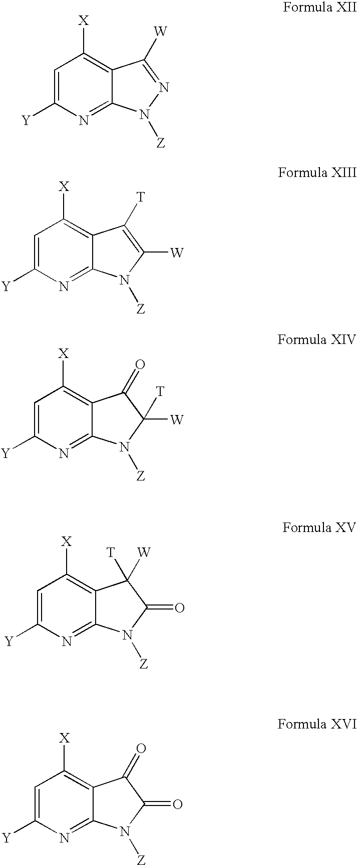 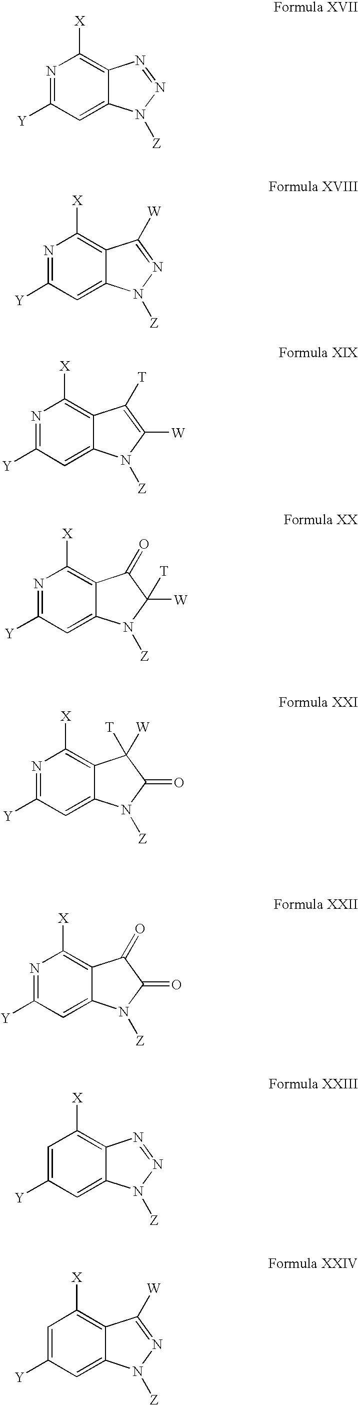 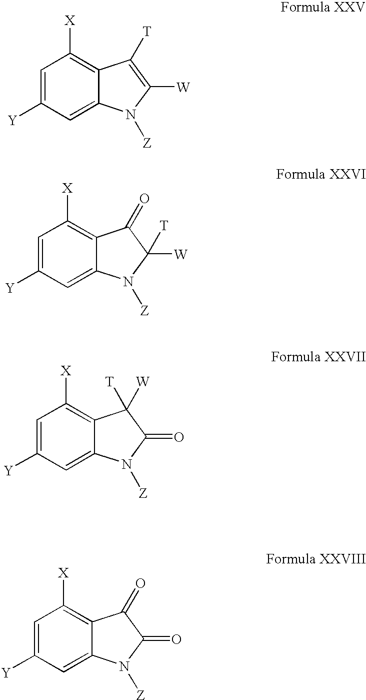
wherein T is H, F, Cl, Br, I, OR₁, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C}.

As disclosed herein, a “pharmaceutically acceptable derivative or prodrug” means any pharmaceutically acceptable salt, ester, salt of an ester or other derivative of a compound of this invention that, upon administration to a recipient, is capable of providing, either directly or indirectly, a compound of the invention or a pharmaceutically active metabolite thereof. Thus, such prodrug is a chemical precursor of a compound of the invention or an active metabolite of a compound of the invention. In one embodiment, such prodrugs may increase the bioavailability of the compounds of this invention when such compounds are administered to a patient (for example, where there is greater absorbance into the blood after oral administration) or which enhance delivery of the parent compound to a biological compartment (e.g., the brain or lymphatic system). Such prodrugs may also demonstrate reduced toxicity, such as where acids in the stomach generate toxic metabolites of compounds of the invention whereas a prodrug passes successfully into the blood prior to metabolism to an active form.

As non-limiting examples, such pharmaceutically acceptable prodrugs include esters, carbonates, thiocarbonates, N-acyl derivatives, N-acyloxyalkyl derivatives, quaternary derivatives of tertiary amines, N-Mannich bases, Schiff bases, aminoacid conjugates, phosphate esters, metal salts and sulfonate esters of the structures disclosed herein according to the invention.

The development of such prodrugs is well known to those of skill in the art. See, for examples, such reference works as: Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38.

The compounds disclosed herein are effective in binding to HSP90. Some relevant data is presented in Table 14 and Table 14A. Effects of compounds on tumor cells are shown in Table 14.

In one embodiment, Y is a halogen, such as F, Cl, Br or I.

In another embodiment, Z is (CR₁₁R₁₂)ₙ—OSO₂NR₉R₁₀ (wherein n=1, 2, 3, 4, 5, 6, 7 or 8). In specific embodiments of such structure, n is between 4 and 8 and R₁₁, R₁₂, R₉, and R₁₀ are each hydrogen.

In another embodiment, Z is (CR₁₁R₁₂)ₙ—NR₁SO₂NR₉R₁₀ (wherein n=1, 2, 3, 4, 5, 6, 7 or 8). In specific embodiments of such structure, n is between 4 and 8 and R₁, R₁₁, R₁₂, R₉, and R₁₀ are each hydrogen.

In another embodiment, Z is (CR₁₁R₁₂)ₙ—NR₁SO₂OH (wherein n=1, 2, 3, 4, 5, 6, 7 or 8). In specific embodiments of such structure, n is between 4 and 8 and R₁, R₁₁, R₁₂ are each hydrogen.

In another embodiment, W is a 6-iodo-benzo[1,3]dioxol-5-yl-methyl:

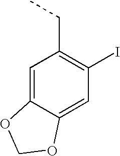

In a specific embodiment, the compounds have the structure of Formula I wherein X is NH₂, Y is F, W is 6-iodo-benzo[1,3]dioxol-5-yl-methyl, and Z is a structural element of the examples provided at table 14 and table 14A. These compounds exhibit an HSP90 binding affinity (see Table 14 and Table 14A).

Synthetic Procedures

A. General Procedures for the Synthesis of Sulfamates (O—SO₂—N), Sulfamides (N—SO₂—N) and N-Substituted Sulfamic Acids (N—SO₂—OH)

In general, primary/secondary/tertiary alcohols and primary/secondary amines are converted to the corresponding sulfamates (O—SO₂—N), sulfamides (N—SO₂—N) and N-substituted sulfamic acids (N—SO₂—OH) by employing methods reported in:

1. Medicinal Chemistry Reviews, Vol. 25, No. 2, 4731-4735 or Tetrahedron 60 (2004) 2187-2190 or J. Med. Chem. 2006, 49, 31-34 (Synthesis procedures for sulfamates and derivatives).

2. Tetrahedron Letters 46, (2005) 4731-4735 (Synthesis of cyclic sulfamates employing i.e. metal catalysts)

3. Organic Letters 2001, Vol. 3, No. 14, 2241-2243 (N-(tert-Butyloxycarbonyl)-N-[4-(dimethylazaniumylidene)-1,4-dihydropyridine-1-ylsulfonyl]azanide for sulfamoylation of amines under very mild conditions to give sulfamide derivatives after deprotection).

4. Synlett 2005, No. 5, 834-836 (Synthesis of cyclic sulfamides).

5. Journal of Organic Chemistry, 57(4), 1252-8; 1992 (Synthesis of N-substituted sulfamic acids (N—SO₂—OH) employing i.e. ClSO₃H)

The corresponding amines and alcohols for derivatization are obtained employing routine reactions for functional group derivatization. Reduction of, e.g., nitriles, ketones, esters, amides and the like, or hydrolysis of, e.g., alkylhalides or esters results in formation of a hydroxy or amino group on the substituent, such as Z. These derivatizations can also be conducted prior to the attachment of said substituent, such as Z, to the core structure of the general formula.
B. Synthesis of Sample Compounds 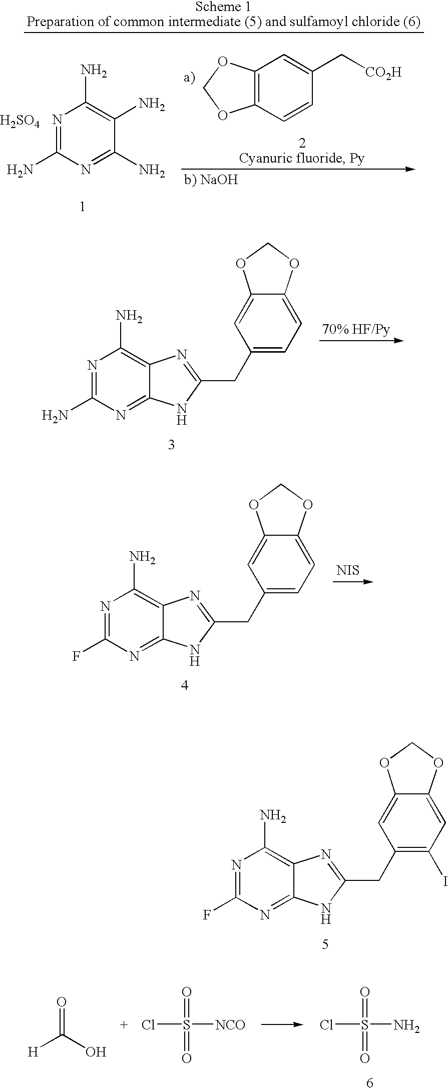
Synthesis Sequence for the Preparation of Intermediate (5) and Sulfamoyl Chloride (6)

### Preparation of Intermediate 3

### 8-(3-[1,3]Dioxolan-4-ylidene-propyl)-9H-purine-2,6-diamine

To a solution of Benzo[1,3]dioxol-5-yl-acetic acid (Compound 2) (3.96 g, 0.022 mol) in DCM (100 mL) was added cyanuric fluoride (2.97 g, 0.022 mol) and pyridine (1.74 g, 0.022 mol). The reaction mixture was stirred at room temperature for 1 hour. Additional DCM (150 mL) was added. The resulting mixture was extracted with H₂O (30 mL), and the acid fluoride was obtained after removal of the solvent under vacuum. The acid fluoride was taken up in DMF (50 mL) and used in the next step.

Compound 1 (4.5 g) was dissolved in H₂O (200 mL) containing NaOH (2.28 g, 0.057 mol). The reaction mixture was heated to 70° C. and the solution of acid fluoride in DMF (50 mL) was added dropwise over 20 min. The reaction mixture was stirred at the same temperature for 1.5 h, cooled to room temperature and concentrated to dryness. To the residue were added MeOH (100 mL) and 25% solution NaOMe/MeOH (100 mL), and the reaction mixture was heated at 90° C. for 18 h. After cooling to room temperature, the pH of the solution was adjusted to 7.0 by addition of conc. HCl. The aqueous layer was removed and the organic layer was diluted with DCM (200 mL) and MeOH (200 mL). The undissolved solids were collected by filtration and purified by chromatography on silica gel eluted with DCM in MeOH (20:1) to give compound 3 as white solid (3.0 g, 56%). ¹HNMR (DMSO-d₆): δ 11.91 (s, 1H), 6.86 (m, 2H), 6.75 (d, 1H), 6.47 (s, 2H), 5.98 (s, 2H), 5.56 (s, 2H) 3.90 (s, 2H). MS (ESI): m/z 285 [M+H].

### Preparation of Intermediate 4

### 8-Benzo[1,3]dioxol-5-ylmethyl-2-fluoro-9H-purin-6-ylamine

8-(3-[1,3]Dioxolan-4-ylidene-propyl)-9H-purine-2,6-diamine (intermediate 3) (3.0 g, 10.6 mmol) was dissolved in pyridine (97 mL), and 70% HF-Py (21 mL) was added, followed by addition of t-butyl nitrite (2.70 mL, 22.1 mmol). The reaction mixture was stirred at room temperature for 2 h. CaCO₃ (9.70 g) was added to the reaction mixture, followed by addition of H₂O (210 mL) and MeOH (140 mL). The resulting mixture was stirred for 12 h, and concentrated in vacuo. MeOH (415 mL)/CH₂Cl₂ (140 mL) was added to the residue. The solid was collected by filtration, and purified by chromatography on silica gel to give title compound 4 as yellow solid (1.66 g, 55%). ¹H NMR (DMSO-d₆): δ 12.86 (b, 1H), 7.59 (b, 2H), 6.88 (m, 2H), 6.76 (m, 1H), 5.99 (s, 2H), 4.01 (s, 2H). MS (ESI): m/z 286 [M−H].

### Preparation of Intermediate 5

### 2-Fluoro-8-(6-iodo-benzo[1,3]dioxol-5-ylmethyl)-9H-purin-6-ylamine

A solution of 8-Benzo[1,3]dioxol-5-ylmethyl-2-fluoro-9H-purin-6-ylamine (compound 4) (1.13 g, 3.9 mmol), NIS (2.12 g, 9.4 mmol) and TFA (450 mL, 3.9 mmol) in CH₂Cl₂ (4.5 mL) was stirred at room temperature overnight. The reaction mixture was concentrated in vacuo to give crude compound 5, which was further purified by chromatography on silica gel eluted with CHCl₃/EtOAc (2:1 to 1:1) to give title compound 5 as a white solid (371 mg, 23% yield) ¹HNMR (CD₃OD): δ 7.32 (s, 1H), 6.92 (s, 1H), 6.00 (s, 2H), 4.24 (s, 2H). MS (ESI): m/z 414 [M+H].

### Preparation of Sulfamoyl Chloride (6)

Formic acid (0.92 g, 20 mmol) was added dropwise to chlorosulfonyl isocyanate (2.83 g, 20 mmol) at 0° C. The reaction mixture was warmed to room temperature and stirred for 1 h. The solvent was evaporated in vacuo to give compound 6 as white solid (2.3 g, 100% yield).

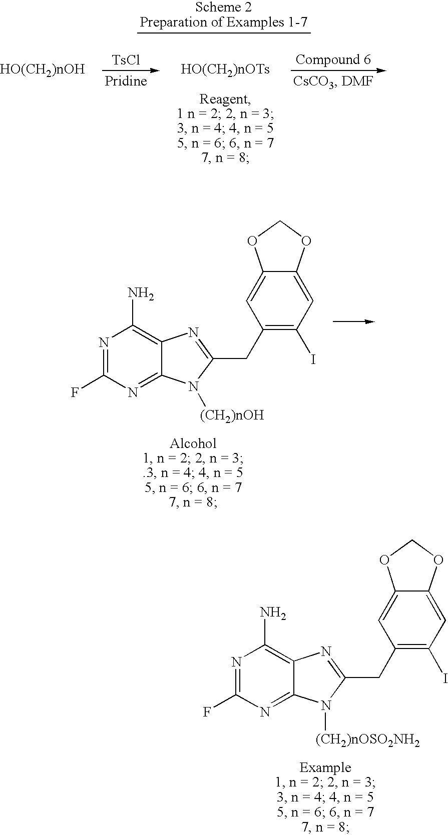

### EXAMPLE 1

### Sulfamic Acid 2-[6-amino-2-fluoro-8-(6-iodo-benzo[1,3]dioxol-5-ylmethyl)-purin-9-yl]-ethyl ester

### Toluene-4-Sulfonic Acid 2-Hydroxy-Ethyl Ester (Reagent 1)

A mixture of HO(CH₂)₂OH, (2.5 g, 40 mmol), TsCl (1.90 g, 10 mmol), pyridine (1.1 g, 12 mmol) and DMAP (12 mg, 0.1 mmol) was stirred at room temperature for 1.5 h. The reaction mixture was partitioned between CH₂Cl₂ (20 mL) and 0.5 M hydrochloric acid (10 mL×2). The organic layer was dried over Na₂ SO₄, and concentrated in vacuo. The resultant residue was purified by chromatography on silica gel (eluted with petroleum ether:ethyl acetate=4:1) to give reagent 1 as colorless oil (1.2 g, 56% yield).

### 2-[6-Amino-2-fluoro-8-(6-iodo-benzo[1,3]dioxol-5-ylmethyl)-purin-9-yl]-ethanol

A mixture of 2-Fluoro-8-(6-iodo-benzo[1,3]dioxol-5-ylmethyl)-9H-purin-6-ylamine (compound 5) (40 mg, 0.1 mmol), TsO(CH₂)₂ OH (compound 7a, 160 mg) and Cs₂CO₃ (40 mg, 0.12 mmol) in anhydrous DMF (1 mL) was heated at 60° C. for 3 h. TLC showed the reaction was complete. The solvent was removed under reduced pressure, and the residue was purified by chromatography on silica gel (eluted with EtOAc/hexanes/CHCl₃/i-PrOH=10:10:10:1) to give crude compound 8.1a (15 mg), which was used in the next step without further purification.

### Sulfamic Acid 2-[6-amino-2-fluoro-8-(6-iodo-benzo[1,3]dioxol-5-ylmethyl)-purin-9-yl]-ethyl ester

A mixture of 2-[6-Amino-2-fluoro-8-(6-iodo-benzo[1,3]dioxol-5-ylmethyl)-purin-9-yl]-ethanol (15 mg), sulfamoyl chloride (20 mg, 0.173 mmol) and CaCO₃ (20 mg, 0.20 mmol) in anhydrous DMF (0.2 mL) at rt for 2 h. TLC indicated that the reaction was complete. The solvent was removed under reduced pressure, and the product was purified by chromatography on silica gel (eluted with EtOAc/hexanes/CHCl₃/i-PrOH=10:10:10:1) to give −1b (1.0 mg,). ¹H NMR (CD₃OD): δ 7.34 (s, 1H), 6.93 (s, 1H), 6.01 (s, 2H), 4.55 (t, J=4.8 Hz, 2H), 4.47 (t, J=4.8 Hz, 2H), 4.35 (s, 2H). MS (ESI): m/z 537 [M+H]⁺.

### EXAMPLE 2

### Sulfamic Acid 3-[6-Amino-2-fluoro-8-(6-iodo-benzo[1,3]dioxol-5-ylmethyl)-purin-9-yl]-propyl ester

### Toluene-4-Sulfonic Acid 3-Hydroxy-Propyl Ester (Reagent 2)

A mixture of 1,3-propanediol (3.1 g, 41 mmol), TsCl (1.90 g, 10 mmol), pyridine (1.1 g, 14 mmol) and DMAP (12 mg, 0.1 mmol) was stirred at room temperature for 1.5 h, and partitioned between CH₂Cl₂ (20 mL) and 0.5 M hydrochloric acid (10 mL). The organic phase was washed one more time with 0.5 M hydrochloric acid (10 mL), dried over Na₂SO₄, and concentrated under reduced pressure. The resultant residue was purified by column chromatography on silica gel (eluted with petroleum ether:ethyl acetate=4:1) to give reagent 2 as a colorless oil (1.5 g, yield: 64%).

### 3-[6-Amino-2-fluoro-8-(6-iodo-benzo[1,3]dioxol-5-ylmethyl)-purin-9-yl]-propan-1-ol

A mixture of 2-Fluoro-8-(6-iodo-benzo[1,3]dioxol-5-ylmethyl)-9H-purin-6-ylamine (intermediate 5) (40 mg, 0.1 mmol), Cs₂CO₃ (60 mg, 0.18 mmol), and toluene-4-sulfonic acid 3-hydroxy-propyl ester (reagent 2, 65 mg, 0.240 mmol) in anhydrous DMF (0.5 mL) was stirred at 60° C. for 2.5 h. The solvent was removed under reduced pressure, and the product was purified by chromatography on silica gel (eluted with EtOAc/hexanes/CHCl₃/i-PrOH=10:10:10:1) to give the crude compound as a colorless oil (15 mg, yield: 33%), which was used in the next step without further purification.

### Sulfamic Acid 3-[6-Amino-2-fluoro-8-(6-iodo-benzo[1,3]dioxol-5-ylmethyl)-purin-9-yl]-propyl ester

A solution of 3-[6-Amino-2-fluoro-8-(6-iodo-benzo[1,3]dioxol-5-ylmethyl)-purin-9-yl]-propan-1-ol (10 mg, 0.021 mmol), CaCO₃ (20 mg, 0.200 mmol), and sulfamoyl chloride (20 mg, 0.173 mmol) in anhydrous DMF (0.2 mL) was stirred at rt for 2 h. The solvent was removed under reduced pressure, and the product was purified by column chromatography on silica gel (eluted with EtOAc/hexanes/CHCl₃/i-PrOH=10:10:10:1) to give −2b as a white solid (1.5 mg, yield: 10%). ¹H NMR (CD₃OD): δ 7.90 (s, 1H), 7.38 (s, 1H), 7.01 (s, 1H), 6.04 (s, 2H), 4.44 (s, 2H), 4.42 (t, J=7.2 Hz, 2H), 4.24 (t, J=6.4 Hz, 2H), 2.34 (m, 2H). MS (ESI): m/z 551 [M+H]⁺.

### EXAMPLE 3

### Sulfamic Acid 4-[6-amino-2-fluoro-8-(6-iodo-benzo[1,3]dioxol-5-ylmethyl)-purin-9-yl]-butyl ester

### Toluene-4-sulfonic acid 4-hydroxy-butyl ester (Reagent 3)

To a mixture of 1,4-Butanediol (1.1 g, 12 mmol) and pyridine (1.8 g, 23 mmol) in CH₂Cl₂ (10 mL) was added dropwise a solution of TsCl (1.9 g, 10 mmol) in CH₂Cl₂ (10 mL). The mixture was stirred at room temperature overnight, and then washed with 0.5 M hydrochloride (5 mL×2), saturated NaHCO₃ (5 mL), and dried over Na₂SO₄. The solvent was removed under reduced pressure, and the resultant residue was purified by column chromatography on silica gel (eluted with petroleum ether:ethyl acetate=4:1) to give reagent 3 as a colorless oil.

### 4-[6-Amino-2-fluoro-8-(6-iodo-benzo[1,3]dioxol-5-ylmethyl)-purin-9-yl]-butan-1-ol

A solution of 2-Fluoro-8-(6-iodo-benzo[1,3]dioxol-5-ylmethyl)-9H-purin-6-ylamine, (40 mg, 0.1 mmol), Cs₂CO₃(60 mg, 0.18 mmol), and reagent 3 (115 mg, 0.47 mmol) in anhydrous DMF (0.7 mL) was stirred at 60° C. for 3 h. The solvent was removed under reduced pressure, and the crude product was purified by chromatography on silica gel (eluted with EtOAc/hexanes/CHCl₃/i-PrOH=10:10:10:1) to give the compound as a white solid.

### Sulfamic Acid 4-[6-amino-2-fluoro-8-(6-iodo-benzo[1,3]dioxol-5-ylmethyl)-purin-9-yl]-butyl ester

A solution of 4-[6-Amino-2-fluoro-8-(6-iodo-benzo[1,3]dioxol-5-ylmethyl)-purin-9-yl]-butan-1-ol (9.7 mg, 0.02 mmol), CaCO₃ (20 mg, 0.20 mmol), and sulfamoyl chloride (20 mg, 0.173 mmol) in anhydrous DMF (0.5 mL) was stirred at room temperature for 2 h. The solvent was removed under reduced pressure, and the crude product was purified by chromatography on silica gel (eluted with EtOAc/hexanes/CHCl₃/i-PrOH=10:10:10:1) to give the title compound as a white solid.

### EXAMPLE 4

### Sulfamic Acid 5-[6-amino-2-fluoro-8-(6-iodo-benzo[1,3]dioxol-5-ylmethyl)-purin-9-yl]-pentyl ester

### Toluene-4-sulfonic acid 5-hydroxy-pentyl ester (Reagent 4)

To a mixture of 1,5-pentanediol (1.2 g, 12 mmol) and pyridine (1.8 g, 23 mmol) in CH₂Cl₂ (10 mL) was added dropwise to a solution of TsCl (1.9 g, 10 mmol) in CH₂Cl₂ (10 mL). The mixture was stirred at room temperature overnight, and then washed with 0.5 M hydrochloride (5 mL×2), saturated NaHCO₃ (5 mL), and dried over Na₂SO₄. The solvent was removed under reduced pressure, and the resultant residue was purified by column chromatography on silica gel (eluted with petroleum ether:ethyl acetate=4:1) to give title compound 7d as a colorless oil (1.2 g, 46% yield).

### 5-[6-Amino-2-fluoro-8-(6-iodo-benzo[1,3]dioxol-5-ylmethyl)-purin-9-yl]-pentan-1-ol

A solution of 2-Fluoro-8-(6-iodo-benzo[1,3]dioxol-5-ylmethyl)-9H-purin-6-ylamine, (intermediate 5, 40 mg, 0.1 mmol), Cs₂CO₃ (60 mg, 0.18 mmol), and reagent 4 (120 mg, 0.47 mmol) in anhydrous DMF (0.7 mL) was stirred at 60° C. for 3 h. The solvent was removed under reduced pressure, and the crude product was purified by chromatography on silica gel (eluted with EtOAc/hexanes/CHCl₃/i-PrOH=10:10:10:1) to give the crude compound as a white solid (20 mg, yield: 42%).

### Sulfamic Acid 5-[6-amino-2-fluoro-8-(6-iodo-benzo[1,3]dioxol-5-ylmethyl)-purin-9-yl]-pentyl ester

A solution of 5-[6-Amino-2-fluoro-8-(6-iodo-benzo[1,3]dioxol-5-ylmethyl)-purin-9-yl]-pentan-1-ol (10 mg, 0.02 mmol), CaCO₃(20 mg, 0.20 mmol), and sulfamoyl chloride (20 mg, 0.173 mmol) in anhydrous DMF (0.5 mL) was stirred at room temperature for 2 h. The solvent was removed under reduced pressure, and the crude product was purified by chromatography on silica gel (eluted with EtOAc/hexanes/CHCl₃/i-PrOH=10:10:10:1) to give the title compound as a white solid (7.1 mg, yield: 61%). ¹H NMR (CD₃OD): δ 7.42 (s, 1H), 7.08 (s, 1H), 6.07 (s, 2H), 4.55 (s, 2H), 4.36 (t, J=7.2 Hz, 2H), 4.14 (t, J=6.0 Hz, 2H), 1.96 (m, 2H), 1.81 (m, 2H), 1.57 (m, 2H). MS (ESI): m/z 579 [M+H]⁺.

### EXAMPLE 5

### Sulfamic Acid 6-[6-amino-2-fluoro-8-(6-iodo-benzo[1,3]dioxol-5-ylmethyl)-purin-9-yl]-hexyl ester

### Toluene-4-sulfonic acid 6-hydroxy-hexyl ester (Reagent 5)

To a mixture of 1,6-hexanediol (2.4 g, 20 mmol) and pyridine (1.8 g, 23 mmol) in CH₂Cl₂ (30 mL) was added dropwise to a solution of TsCl (1.9 g, 10 mmol) in CH₂Cl₂ (10 mL). The mixture was stirred at rt overnight, and washed with 1.0 M hydrochloride (10 mL×2) and dried over Na₂SO₄. After the solvent was removed under reduced pressure, the residue was purified by chromatography on silica gel (eluted with petroleum ether:ethyl acetate=4:1) to give compound 7e as a colorless oil (1.26 g, yield: 46%)

### 6-[6-Amino-2-fluoro-8-(6-iodo-benzo[1,3]dioxol-5-ylmethyl)-purin-9-yl]-hexan-1-ol

A solution of 2-Fluoro-8-(6-iodo-benzo[1,3]dioxol-5-ylmethyl)-9H-purin-6-ylamine (intermediate 5) (60 mg, 0.145 mmol), Cs₂CO₃ (60 mg, 0.18 mmol), and reagent 5 (195 mg, 0.714 mmol) in anhydrous DMF (0.6 mL) was stirred at 60° C. for 3 h. The solvent was removed under reduced pressure, and the product was purified by column chromatography on silica gel (eluted with EtOAc/hexanes/CHCl₃/i-PrOH=10:10:10:1) to give crude compound as a white solid (20 mg, yield: 27%).

### Sulfamic Acid 6-[6-amino-2-fluoro-8-(6-iodo-benzo[1,3]dioxol-5-ylmethyl)-purin-9-yl]-hexyl ester

A solution of 6-[6-Amino-2-fluoro-8-(6-iodo-benzo[1,3]dioxol-5-ylmethyl)-purin-9-yl]-hexan-1-(15 mg, 0.029 mmol), CaCO₃ (20 mg, 0.20 mmol), and sulfamoyl chloride (15 mg, 0.13 mmol) in anhydrous DMF (0.5 mL) was stirred at room temperature for 2 h. The mixture was concentrated in vacuo, and the crude product was purified by chromatography on silica gel (eluted with EtOAc/hexanes/CHCl₃/i-PrOH=10:10:10:1) to give −5b as a white solid (2.1 mg, yield: 10%). ¹H NMR (CD₃OD): δ 7.35 (s, 1H), 6.88 (s, 1H), 6.00 (s, 2H), 4.29 (s, 2H), 4.15 (t, J=7.6 Hz, 2H), 4.10 (t, J=6.0 Hz, 2H), 1.78 (m, 2H), 1.70 (m, 2H), 1.44 (m, 4H). MS (ESI): m/z 593 [M+H]⁺.

### EXAMPLE 6

### Sulfamic Acid 7-[6-amino-2-fluoro-8-(6-iodo-benzo[1,3]dioxol-5-ylmethyl)-purin-9-yl]-heptyl ester

### Toluene-4-sulfonic acid 7-hydroxy-heptyl ester (Reagent 6)

To a mixture of 1,7-heptanediol (1.56 g, 12 mmol) and pyridine (0.91 g, 10 mmol) in CH₂Cl₂ (20 mL) was added dropwise to a solution of TsCl (1.9 g, 10 mmol) in CH₂Cl₂ (10 mL). The mixture was stirred at rt overnight, and washed with 1.0 M hydrochloride (10 mL×2) and dried over Na₂SO₄. After the solvent was removed under reduced pressure, the residue was purified by chromatography on silica gel (eluted with petroleum ether:ethyl acetate=4:1) to give reagent 6 as a colorless oil (0.84 g, yield 30%).

### 7-[6-Amino-2-fluoro-8-(6-iodo-benzo[1,3]dioxol-5-ylmethyl)-purin-9-yl]-heptan-1-ol

A mixture of 2-Fluoro-8-(6-iodo-benzo[1,3]dioxol-5-ylmethyl)-9H-purin-6-ylamine (intermediate 5) (40 mg, 0.10 mmol), TsO(CH₂)₇OH (160 mg) and Cs₂CO₃ (40 mg, 0.12 mmol) in DMF (1 mL) was heated at 60° C. for 3 h. TLC showed the reaction was complete. The solvent was removed under reduced pressure, and the residue was purified by chromatography on silica gel (eluted with EtOAc/hexanes/CHCl₃/i-PrOH=10:10:10:1) to give compound (10 mg), which was used in the next reaction without further purification.

### Sulfamic Acid 7-[6-amino-2-fluoro-8-(6-iodo-benzo[1,3]dioxol-5-ylmethyl)-purin-9-yl]-heptyl ester

A mixture of 7-[6-Amino-2-fluoro-8-(6-iodo-benzo[1,3]dioxol-5-ylmethyl)-purin-9-yl]-heptan-1-ol (10 mg), sulfamoyl chloride (20 mg, 0.17 mmol) and CaCO₃ (20 mg, 0.20 mmol) in anhydrous DMF (0.2 mL) at room temperature for 2 h. TLC indicated the reaction was complete. The mixture was concentrated in vacuo, and the crude product was purified by chromatography on silica gel (eluted with EtOAc/hexanes/CHCl₃/i-PrOH=10:10:10:1) to give the compound as a white solid (1.2 mg, HPLC 90%). ¹H NMR (CD₃OD): δ 7.29 (s, 1H), 6.81 (s, 1H), 5.94 (s, 2H), 4.24 (s, 2H), 4.08 (t, J=7.6 Hz, 2H), 4.03 (t, J=6.4 Hz, 2H), 1.70 (m, 2H), 1.63 (m, 2H), 1.32 (m, 6H). MS (ESI): m/z 607 [M+H]⁺.

### EXAMPLE 7

### Sulfamic Acid 8-[6-amino-2-fluoro-8-(6-iodo-benzo[1,3]dioxol-5-ylmethyl)-purin-9-yl]-octyl ester

### Toluene-4-sulfonic acid 8-hydroxy-octyl ester (Reagent 7)

To a mixture of HO(CH₂)₈OH (1.8 g, 12 mmol) and pyridine (0.91 g, 10 mmol) in CH₂Cl₂ (20 mL) was added dropwise a solution of TsCl (1.9 g, 10 mmol) in CH₂Cl₂ (10 mL). The mixture was stirred at rt overnight, and washed with 1.0 M hydrochloride (10 mL×2) and dried over Na₂SO₄. After the solvent was removed under reduced pressure, the residue was purified by chromatography on silica gel (eluted with petroleum ether:ethyl acetate=4:1) to give reagent 7 as a colorless oil (1.3 g, 43% yield).

### 8-[6-Amino-2-fluoro-8-(6-iodo-benzo[1,3]dioxol-5-ylmethyl)-purin-9-yl]-octan-1-ol

A mixture of intermediate 5 (20 mg, 0.05 mmol), TsO(CH₂)₈OH (72 mg, 0.24 mmol) and Cs₂CO₃ (19 mg, 0.057 mmol) in DMF (0.5 mL) was heated at 60° C. for 3 h. TLC showed the reaction was complete. The solvent was removed under reduced pressure, and the residue was purified by chromatography on silica gel (eluted with EtOAc/hexanes/CHCl₃/i-PrOH=10:10:10:1) to give the compound as a white solid (5 mg, 13% yield).

### Sulfamic Acid 8-[6-amino-2-fluoro-8-(6-iodo-benzo[1,3]dioxol-5-ylmethyl)-purin-9-yl]-octyl ester

A mixture of 8-[6-Amino-2-fluoro-8-(6-iodo-benzo[1,3]dioxol-5-ylmethyl)-purin-9-yl]-octan-1-ol (19 mg, 0.035 mmol), sulfamoyl chloride (14 mg, 0.12 mmol) and CaCO₃ (10 mg, 0.10 mmol) in anhydrous DMF (0.2 mL) was stirred at room temperature for 2 h. The mixture was concentrated in vacuo, and the crude product was purified by chromatography on silica gel (eluted with EtOAc/hexanes/CHCl₃/i-PrOH=10:10:10:1) to give the title compound as a white solid (5 mg, yield: 20%, HPLC 90%). ¹H NMR (DMSO-d₆): δ 7.61 (b, 1H), 7.39 (s, 1H), 7.39 (b, 1H), 6.84 (s, 1H), 6.02 (s, 2H), 4.20 (s, 2H), 3.98 (m, 4H), 1.59 (m, 4H), 1.22 (m, 10H). MS (ESI): m/z 607 [M+H]⁺.

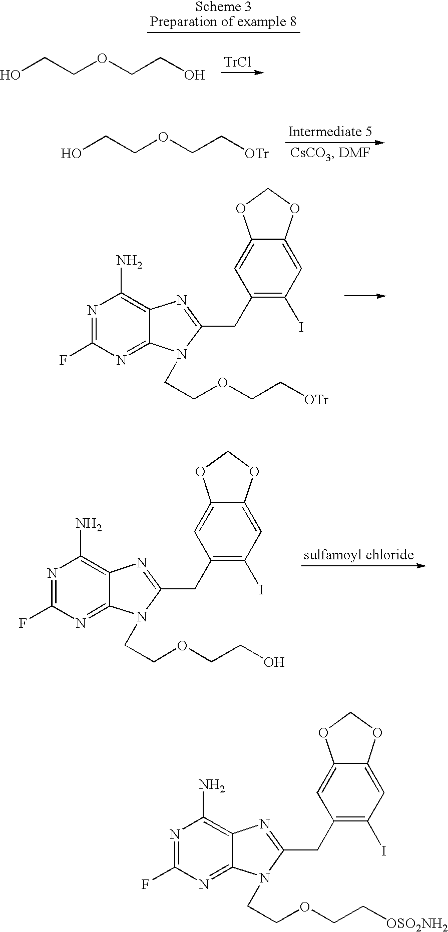

### EXAMPLE 8

### Sulfamic Acid 2-{2-[6-amino-2-fluoro-8-(6-iodo-benzo[1,3]dioxol-5-ylmethyl)-purin-9-yl]-ethoxy}-ethyl ester

### 2-(2-Trityloxy-ethoxy)-ethanol

2-(2-Hydroxy-ethoxy)-ethanol (2.12 g, 20 mmol), trityl chloride (2.78 g, 10 mmol) and TEA (5 mL, 36 mmol) was dissolved in DCM (15 mL) at 0° C. The reaction mixture was slowly warmed to room temperature and stirred overnight. The solvent was removed under vacuum and the crude product was purified by chromatography on silica gel (eluted with Hexane/EtOAc=4:1 to 2:1) to give the compound as a white solid (2.2 g, 63% yield).

### 2-Fluoro-8-(6-iodo-benzo[1,3]dioxol-5-ylmethyl)-9-[2-(2-trityloxy-ethoxy)-ethyl]-9H-purin-6-ylamine

Intermediate 5 (35 mg, 0.085 mmol), DBAD (38.8 mg, 0.17 mmol), triphenyl-phosphine (44 mg, 0.17 mmol) and 2-(2-trityloxy-ethoxy)-ethanol (20 mg, 0.17 mmol) was dissolved in 2 mL of DCM and toluene (1:5). The reaction mixture was stirred at room temperature overnight and concentrated in vacuo. The residue was purified by preparative TLC (eluted with EtOAc/Toluene=1:2) to give compound 33 (28 mg, 44% yield).

### 2-{2-[6-Amino-2-fluoro-8-(6-iodo-benzo[1,3]dioxol-5-ylmethyl)-purin-9-yl]-ethoxy}-ethanol

2-Fluoro-8-(6-iodo-benzo[1,3]dioxol-5-ylmethyl)-9-[2-(2-trityloxy-ethoxy)-ethyl]-9H-purin-6-ylamine (28 mg, 0.038 mmol) was added CF₃COOH/CH₂Cl₂ (0.5 mL, 1:1). The reaction mixture was stirred at room temperature for 4 hours, and the solvent was removed under reduced pressure. The residue was purified by preparative TLC (eluted with CHCl₃/MeOH=15:1) to give the compound (11 mg, 58% yield).

### Sulfamic Acid 2-{2-[6-amino-2-fluoro-8-(6-iodo-benzo[1,3]dioxol-5-ylmethyl)-purin-9-yl]-ethoxy}-ethyl ester

2-{2-[6-Amino-2-fluoro-8-(6-iodo-benzo[1,3]dioxol-5-ylmethyl)-purin-9-yl]-ethoxy}-ethanol (11 mg, 0.022 mmol), ClSO₂NH₂ (12 mg, 0.10 mmol) was dissolved in anhydrous DMF (1 mL). CaCO₃ (13 mg, 0.13 mmol) was added and the reaction mixture was stirred at room temperature for 4 hours. The solvent was removed and the residue was purified with preparative TLC (eluted with CHCl₃/MeOH=15:1) to give the desired compound (9 mg, 71% yield).

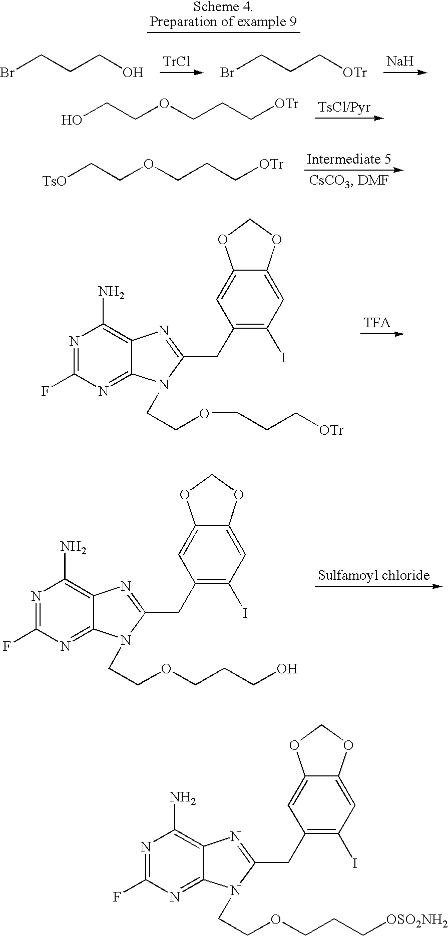

### EXAMPLE 9

### Sulfamic Acid 3-{2-[6-amino-2-fluoro-8-(6-iodo-benzo[1,3]dioxol-5-ylmethyl)-purin-9-yl]-ethoxy}-propyl ester

### 1-Bromo-3-trityloxy-propane

3-bromo-propanol (7.2 g, 52 mmol), trityl chloride (15.1 g, 54 mmol) and TEA (7.7 mL, 55 mmol) was solved in DCM (60 mL) at 0° C. The reaction mixture was slowly warmed to room temperature and stirred overnight. The solvent was removed under vacuum and the crude product was purified by chromatography on silica gel (eluted with Hexane/EtOAc=2:1 to 1:1) to give the compound as a white solid (13.3 g, 67% yield).

### 2-(3-Trityloxypropyloxy)-ethanol

To a mixture of ethylene glycol (12 mL) in DMF (40 mL) at 0° C. was added NaH (2.4 g, 100 mmol) slowly. The reaction mixture was stirred for 30 minutes until no gas evolved. 1-Bromo-3-trityloxy-propane (7.6 g, 20 mmol) was added and the reaction mixture was warmed to room temperature and stirred overnight. The reaction mixture was partitioned between DCM and H₂O, and the crude product was purified by chromatography on silica gel (eluted with Hexane/EtOAc=2:1 to 1:1) to give the compound as colorless oil (5.6 g, 77% yield).

### Toluene-4-sulfonic acid 2-(3-trityloxy-propoxy)-ethyl ester

2-(3-Trityloxypropyloxy)-ethanol (365 mg, 1 mmol) and TsCl (210 mg, 1.1 mmol) was dissolved in pyridine (2 mL) at 0° C. The reaction mixture was warmed to room temperature overnight. The solvent was removed under vacuum and the crude product was purified by chromatography on silica gel (eluted with Hexane/EtOAc=10:1 to 2:1) to give the compound as colorless oil (470 mg, 91% yield).

### 2-Fluoro-8-(6-iodo-benzo[1,3]dioxol-5-ylmethyl)-9-[2-(3-trityloxy-propoxy)-ethyl]-9H-purin-6-ylamine

Intermediate 5 (40 mg, 0.097 mmol), Cs₂CO₃ (60 mg, 0.184 mmol) and Toluene-4-sulfonic acid 2-(3-trityloxy-propoxy)-ethyl ester (88 mg, 0.17 mmol) was dissolved in anhydrous DMF (1 mL). The reaction mixture was heated to 70° C. (oil temperature) with stir for 8 hours. The solvent was removed under vacuum completely and the residue was purified by preparative TLC (eluted with toluene/EtOAc=2:1) to give 28 mg the desired compound as white solid.

### 3-{2-[6-Amino-2-fluoro-8-(6-iodo-benzo[1,3]dioxol-5-ylmethyl)-purin-9-yl]-ethoxy}-propan-1-ol

3-{2-[6-Amino-2-fluoro-8-(6-iodo-benzo[1,3]dioxol-5-ylmethyl)-purin-9-yl]-ethoxy}-propyloxy-1-trityl (28 mg, 0.037 mmol) was dissolved in CF₃COOH/CH₂Cl₂(0.5 mL, 1:1). The reaction mixture was stirred at room temperature for 4 hours, and concentrated in vacuum. The residue was purified with preparative TLC (eluted with CHCl₃/MeOH=15:1) to give 8.9a (9 mg, 46% yield).

### Sulfamic Acid 3-{2-[6-amino-2-fluoro-8-(6-iodo-benzo[1,3]dioxol-5-ylmethyl)-purin-9-yl]-ethoxy}-propyl ester

3-{2-[6-Amino-2-fluoro-8-(6-iodo-benzo[1,3]dioxol-5-ylmethyl)-purin-9-yl]-ethoxy}-propan-1-ol (9 mg, 0.017 mmol), ClSO₂NH₂ (10 mg, 0.086 mmol) was dissolved in anhydrous DMF (1 mL). CaCO₃ (11 mg 0.11 mmol) was added and the reaction mixture was stirred at room temperature for 4 hours. The solvent was removed under reduced pressure and the residue was purified by preparative TLC (eluted with CHCl₃/MeOH=15:1) to give 8.9b (7 mg, 67% yield).

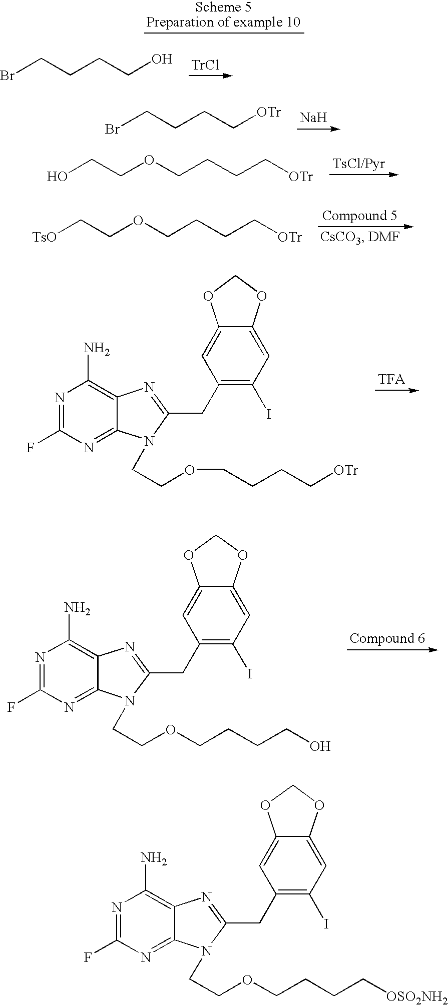

### EXAMPLE 10

### Sulfamic Acid 4-{2-[6-amino-2-fluoro-8-(6-iodo-benzo[1,3]dioxol-5-ylmethyl)-purin-9-yl]-ethoxy}-butyl ester

### 1-Bromo-trityloxybutane

1-Bromo-4-butanol (2.50 g, 16.3 mmol), trityl chloride (6.83 g, 24.5 mmol) and TEA (3.4 mL, 24.5 mmol) was dissolved in DCM (30 mL) at 0° C. The reaction mixture was slowly warmed to room temperature and stirred overnight. The solvent was removed under vacuum and the crude product was purified by chromatography on silica gel (eluted with Hexane/EtOAc=2:1 to 1:1) to give the compound as colorless oil (5.2 g, 81% yield).

### 2-(4-Trityloxybutyoxy)-ethanol

To a mixture of ethylene glycol (4 mL) in 20 mL DMF (20 mL) at 0° C. was added NaH (2.0 g of 60%, 50 mmol) slowly, and the reaction mixture was stirred for 30 minutes until no gas evolved. 1-Bromo-trityloxybutane (1.93 g, 4.88 mmol) was added and the reaction was warmed to room temperature overnight. The reaction mixture partitioned between DCM and H₂O, and the crude product was purified by chromatography on silica gel (eluted with hexane to ethyl acetate 2:1 to 1:1) to give the compound as colorless oil (1.43 g, 76% yield).

### Toluene-4-sulfonic acid 2-(4-trityloxy-butoxy)-ethyl ester

2-(4-Trityloxybutyoxy)-ethanol (0.246 g, 6.53 mmol) and TsCl (0.15 g, 7.84 mmol) was dissolved in pyridine (3 mL) at 0° C. The reaction mixture was warmed to room temperature overnight, and concentrated in vacuo. The crude product was purified by chromatography on silica gel (eluted with Hexane/EtOAc=4:1) to give the compound as colorless oil (118 mg, 34% yield).

### 2-Fluoro-8-(6-iodo-benzo[1,3]dioxol-5-ylmethyl)-9-[2-(4-trityloxy-butoxy)-ethyl]-9H-purin-6-ylamine

Intermediate 5 (60 mg, 0.145 mmol), Cs₂CO₃ (100 mg, 0.307 mmol) and 2-(4-Trityloxybutyloxy)-ethoxy-2′-trityl (118 mg, 0.222 mmol) was dissolved in anhydrous DMF (1.5 mL). The reaction mixture was heated to 70° C. (oil temperature) with stir for 8 hours. The solvent was removed under vacuum completely and the residue was purified by preparative TLC (Toluene/EtOAc=2:1) to give the compound as light yellow foam (58 mg, yield 52%).

### 4-{2-[6-Amino-2-fluoro-8-(6-iodo-benzo[1,3]dioxol-5-ylmethyl)-purin-9-yl]-ethoxy}-butan-1-ol

2-Fluoro-8-(6-iodo-benzo[1,3]dioxol-5-ylmethyl)-9-[2-(4-trityloxy-butoxy)-ethyl]-9H-purin-6-ylamine (25 mg, 0.032 mmol) was dissolved in CF₃COOH/CH₂Cl₂ (0.5 mL, 1:1). The reaction mixture was stirred at room temperature for 4 h, concentrated in vacuo and the residue was purified by preparative TLC (eluted with CHCl₃/MeOH=15:1) to give the compound (6 mg, 35% yield).

### Sulfamic Acid 4-{2-[6-amino-2-fluoro-8-(6-iodo-benzo[1,3]dioxol-5-ylmethyl)-purin-9-yl]-ethoxy}-butyl ester

4-{2-[6-Amino-2-fluoro-8-(6-iodo-benzo[1,3]dioxol-5-ylmethyl)-purin-9-yl]-ethoxy}-butan-1-ol (6 mg, 0.011 mmol), ClSO₂NH₂ (6 mg, 0.052 mmol) was dissolved in anhydrous DMF (1 mL). CaCO₃ (7 mg, 7 mmol) was added and the reaction mixture was stirred at room temperature for 4 hours. The solvent was removed under vacuum and the residue was purified by preparative TLC (eluted with CHCl₃/MeOH=15:1) to give the compound (3 mg, 44% yield).

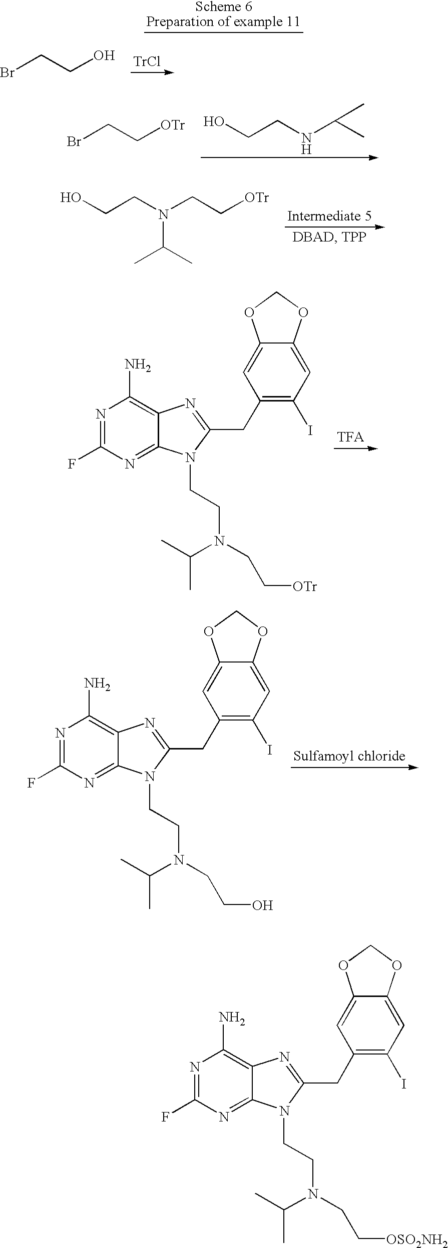

### EXAMPLE 11

### Sulfamic Acid 2-({2-[6-amino-2-fluoro-8-(6-iodo-benzo[1,3]dioxol-5-ylmethyl)-purin-9-yl]-ethyl}-isopropyl-amino)-ethyl ester

### 1-Bromo-2-trityloxy-ethane

2-Bromo-ethanol (0.63 g, 5.0 mmol), Et₃N (1 mL, 7.2 mmol) and trityl chloride (1.46 g, 5.25 mmol) in anhydrous CH₂Cl₂ (5 mL) was stirred at room temperature overnight and concentrated in vacuo. The residue was purified with column chromatography on silica gel (eluted with hexane/EtOAc from 20:1 to 4:1) to give the compound (1.2 g, 82% yield).

### 2-[Isopropyl-(2-trityloxy-ethyl)-amino]-ethanol

1-Bromo-2-trityloxy-ethane (0.734 g, 2.0 mmol), Et₃N (1.0 mL, 7.2 mmol) and 2-isopropylaminoethanol (0.62 g, 6.0 mmol) in anhydrous CH₃CN (5 mL) was refluxed overnight and concentrated in vacuo. The residue was purified with chromatography on silica gel (eluted with CHCl₃/MeOH/NH₃.H₂O=20:1:0.5) to give the compound (0.50 g, 64% yield).

### 2-Fluoro-8-(6-iodo-benzo[1,3]dioxol-5-ylmethyl)-9-{2-[isopropyl-(2-trityloxy-ethyl)-amino]-ethyl}-9H-purin-6-ylamine

Compound 5 (33 mg, 0.080 mmol), DBAD (Di-tert-butyl azodicarboxylate, 36.8 mg, 0.16 mmol), triphenyl-phosphine (42 mg, 0.16 mmol) and 2-[Isopropyl-(2-trityloxy-ethyl)-amino]-ethanol (63 mg, 0.16 mmol) were dissolved in a mixture of DCM and toluene (2 mL, 1:5). The reaction mixture was stirred at room temperature overnight and concentrated in vacuo. The residue was purified by preparative TLC (Toluene/EtOAc=1:1) to give the compound as light yellow foam (22 mg, 34% yield).

### 2-({2-[6-Amino-2-fluoro-8-(6-iodo-benzo[1,3]dioxol-5-ylmethyl)-purin-9-yl]-ethyl}-isopropyl-amino)-ethanol

2-({2-[6-Amino-2-fluoro-8-(6-iodo-benzo[1,3]dioxol-5-ylmethyl)-purin-9-yl]-ethyl}-isopropyl-amino)-ethyloxy-1′-trityl (22 mg, 0.028 mmol) was dissolved in CF₃COOH/CH₂Cl₂ (0.5 mL, 1:1). The reaction mixture was stirred at room temperature for 4 hours, and concentrated in vacuo. The residue was purified by preparative TLC (eluted with CHCl₃/MeOH=10:1) to give the desired compound (6 mg, 40% yield).

### Sulfamic Acid 2-({2-[6-amino-2-fluoro-8-(6-iodo-benzo[1,3]dioxol-5-ylmethyl)-purin-9-yl]-ethyl}-isopropyl-amino)-ethyl ester

2-({2-[6-Amino-2-fluoro-8-(6-iodo-benzo[1,3]dioxol-5-ylmethyl)-purin-9-yl]-ethyl}-isopropyl-amino)-ethanol (6 mg, 0.011 mmol), ClSO₂NH₂ (6 mg, 0.052 mmol) was dissolved in anhydrous DMF (1 mL). CaCO₃ (7 mg, 7 mmol) was added and the reaction mixture was stirred at room temperature for 4 hours, concentrated in vacuo and the residue was purified by preparative TLC (eluted with CHCl₃/MeOH=15:1) to give the desired compound (3 mg, 44% yield).

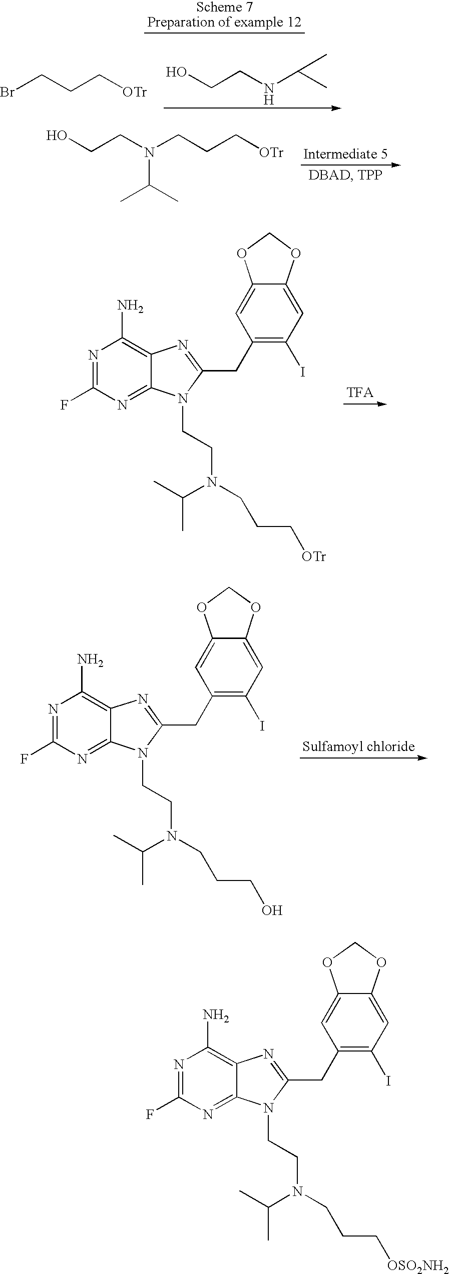

### EXAMPLE 12

### Sulfamic Acid 3-({2-[6-amino-2-fluoro-8-(6-iodo-benzo[1,3]dioxol-5-ylmethyl)-purin-9-yl]-ethyl}-isopropyl-amino)-propyl ester

### 2-[Isopropyl-(3-trityloxy-propyl)-amino]-ethanol

A mixture of 1-Bromo-3-trityloxypropane (3.81 g, 10.0 mmol), Et₃N (2.02 g, 20.0 mmol) and 2-isopropylaminoethanol (3.1 g, 30.0 mmol) in 20 mL of anhydrous CH₃CN was refluxed overnight and concentrated in vacuo. The residue was purified with column chromatography on silica gel (eluted with CHCl₃/MeOH/NH₃.H₂O=20:1:0.5) to give the compound as a brown oil (2.8 g, 69% yield).

### 2-Fluoro-8-(6-iodo-benzo[1,3]dioxol-5-ylmethyl)-9-{2-[isopropyl-(3-trityloxy-propyl)-amino]-ethyl}-9H-purin-6-ylamine

A mixture of intermediate 5 (33 mg, 0.080 mmol), DBAD (36.8 mg, 0.16 mmol), triphenylphosphine (42 mg, 0.16 mmol) and 2-[Isopropyl-(3-trityloxy-propyl)-amino]-ethanol (65 mg, 0.16 mmol) in 2 mL of in DCM and toluene (1:5) was stirred overnight and concentrated in vacuo. The residue was purified by preparative TLC (eluted with toluene/EtOAc=1:1) to give the compound as light yellow foam (30 mg, 48% yield).

### 3-({2-[6-Amino-2-fluoro-8-(6-iodo-benzo[1,3]dioxol-5-ylmethyl)-purin-9-yl]-ethyl}-isopropyl-amino)-propan-1-ol

2-Fluoro-8-(6-iodo-benzo[1,3]dioxol-5-ylmethyl)-9-{2-[isopropyl-(3-trityloxy-propyl)-amino]-ethyl}-9H-purin-6-ylamine (30 mg, 0.038 mmol) was dissolved in CF₃COOH/CH₂Cl₂ (0.5 mL, 1:1). The reaction mixture was stirred at room temperature for 4 hours, concentrated in vacuo, and the residue was purified by preparative TLC (eluted with CHCl₃/MeOH=10:1) to give the desired compound (9 mg, 43% yield).

### Sulfamic Acid 3-({2-[6-amino-2-fluoro-8-(6-iodo-benzo[1,3]dioxol-5-ylmethyl)-purin-9-yl]-ethyl}-isopropyl-amino)-propyl ester

3-({2-[6-Amino-2-fluoro-8-(6-iodo-benzo[1,3]dioxol-5-ylmethyl)-purin-9-yl]-ethyl}-isopropyl-amino)-propan-1-ol (9 mg, 0.016 mmol), ClSO₂NH₂ (9 mg, 0.078 mmol) was dissolved in anhydrous DMF (1 mL). CaCO₃ (10 mg, 10 mmol) was added and the reaction mixture was stirred at room temperature for 4 hours, concentrated in vacuo, and the residue was purified by preparative TLC (eluted with CHCl₃/MeOH=15:1) to give the desired compound (5 mg, 49% yield).

### EXAMPLE 13

### Sulfamic Acid 4-({2-[6-amino-2-fluoro-8-(6-iodo-benzo[1,3]dioxol-5-ylmethyl)-purin-9-yl]-ethyl}-isopropyl-amino)-butyl ester

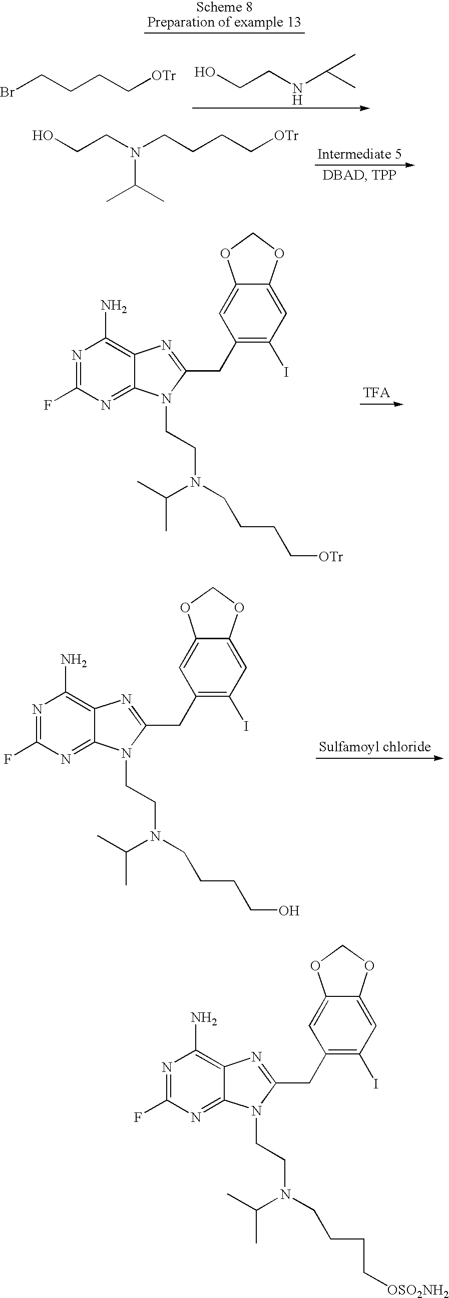

### 2-[Isopropyl-(4-trityl oxy-butyl)-amino]-ethanol

1-Bromo-4-trityloxybutane (1.4 g, 3.54 mmol), Et₃N (0.36 g, 3.54 mmol) and 2-isopropylaminoethanol (1.1 g, 10.6 mmol) in 10 mL of anhydrous CH₃CN was refluxed overnight and concentrated in vacuo. The residue was purified with chromatography on silica gel (eluted with CHCl₃/MeOH/NH₃.H₂O=20:1:0.5) to give the compound as a brown oil (1.1 g, 74% yield).

### 2-Fluoro-8-(6-iodo-benzo[1,3]dioxol-5-ylmethyl)-9-{2-[isopropyl-(4-trityloxy-butyl)-amino]-ethyl}-9H-purin-6-ylamine

Intermediate 5 (33 mg, 0.080 mmol), DBAD (36.8 mg, 0.16 mmol), triphenyl-phosphine (42 mg, 0.16 mmol) and 2-[Isopropyl-(4-trityloxy-butyl)-amino]-ethanol (67 mg, 0.16 mmol) in DCM and toluene (2 mL, 1:5) was stirred overnight and concentrated in vacuo. The residue was purified through preparative TLC (eluted with toluene/EtOAc=1:1) to give the compound as light yellow foam (11 mg, 16% yield).

### 4-({2-[6-Amino-2-fluoro-8-(6-iodo-benzo[1,3]dioxol-5-ylmethyl)-purin-9-yl]-ethyl}-isopropyl-amino)-butan-1-ol

4-({2-[6-Amino-2-fluoro-8-(6-iodo-benzo[1,3]dioxol-5-ylmethyl)-purin-9-yl]-ethyl}-isopropyl-amino)-butyloxy-1′-trityl (11 mg, 0.013 mmol) was dissolved in CF₃COOH/CH₂Cl₂ (0.5 mL, 1:1). The reaction mixture was stirred at room temperature for 4 hours, concentrated in vacuo and the residue was purified by preparative TLC (eluted with CHCl₃/MeOH=10:1) to give the compound (4 mg, 54%)

### Sulfamic Acid 4-({2-[6-amino-2-fluoro-8-(6-iodo-benzo[1,3]dioxol-5-ylmethyl)-purin-9-yl]-ethyl}-isopropyl-amino)-butyl ester

4-({2-[6-Amino-2-fluoro-8-(6-iodo-benzo[1,3]dioxol-5-ylmethyl)-purin-9-yl]-ethyl}-isopropyl-amino)-butan-1-ol (11 mg, 0.078 mmol) was dissolved in anhydrous DMF (1 mL). CaCO₃ (10 mg, 10 mmol) was added and the reaction mixture was stirred at room temperature for 4 hours, concentrated in vacuo, and the residue was purified by preparative TLC (eluted with CHCl₃/MeOH=15:1) to give the desired compound (5 mg, 49% yield).

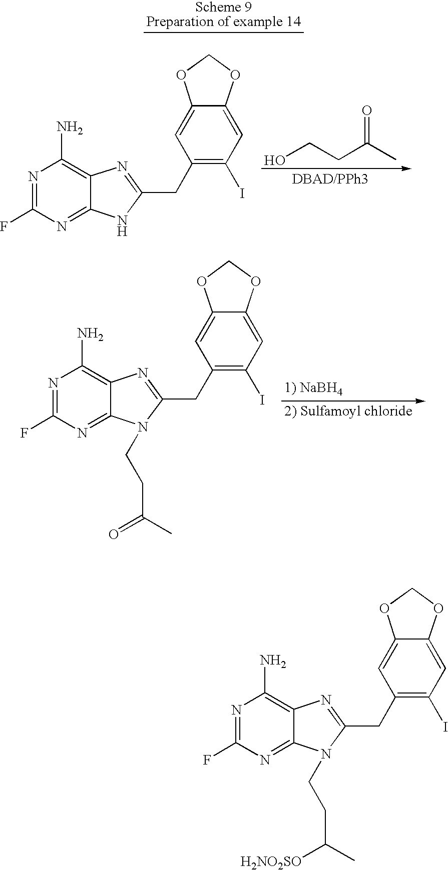

### EXAMPLE 14

### Sulfamic Acid 3-[6-amino-2-fluoro-8-(6-iodo-benzo[1,3]dioxol-5-ylmethyl)-purin-9-yl]-3-methyl-propyl ester

### 4-[6-Amino-2-fluoro-8-(6-iodo-benzo[1,3]dioxol-5-ylmethyl)-purin-9-yl]-butan-2-one

A mixture of intermediate 5 (33 mg, 0.080 mmol), DBAD (36.8 mg, 0.16 mmol), triphenylphosphine (42 mg, 0.16 mmol) and 4-hydroxy-butan-2-one (14 mg, 0.16 mmol) in DCM and toluene (2 mL, 1:5) was stirred overnight and concentrated in vacuo. The residue was purified by preparative TLC (eluted with toluene/EtOAc=3:1) to give the compound (3 mg, 7.8% yield).

### 4-[6-Amino-2-fluoro-8-(6-iodo-benzo[1,3]dioxol-5-ylmethyl)-purin-9-yl]-butan-2-ol

4-[6-Amino-2-fluoro-8-(6-iodo-benzo[1,3]dioxol-5-ylmethyl)-purin-9-yl]-butan-2-one (3 mg, 0.0062 mmol) was dissolved in 1 mL of MeOH at 0° C. NaBH₄ (3 mg, 0.079 mmol) was added and the reaction mixture was stirred at 0° C. for 1 hour. The solvent was removed under reduced pressure and the residue was purified by preparative TLC (eluted with CHCl₃/MeOH=10:1) to give the compound as white solid (2 mg, 66% yield).

### Sulfamic Acid 3-[6-amino-2-fluoro-8-(6-iodo-benzo[1,3]dioxol-5-ylmethyl)-purin-9-yl]-3-methyl-propyl ester

A solution of 4-[6-Amino-2-fluoro-8-(6-iodo-benzo[1,3]dioxol-5-ylmethyl)-purin-9-yl]-butan-2-ol (9.7 mg, 0.02 mmol), CaCO₃ (20 mg, 0.20 mmol), and sulfamoyl chloride (20 mg, 0.173 mmol) in anhydrous DMF (0.5 mL) was stirred at room temperature for 2 h. The solvent was removed under reduced pressure, and the crude product was purified by chromatography on silica gel (eluted with EtOAc/hexanes/CHCl₃/i-PrOH=10:10:10:1) to give the title compound as a white solid.

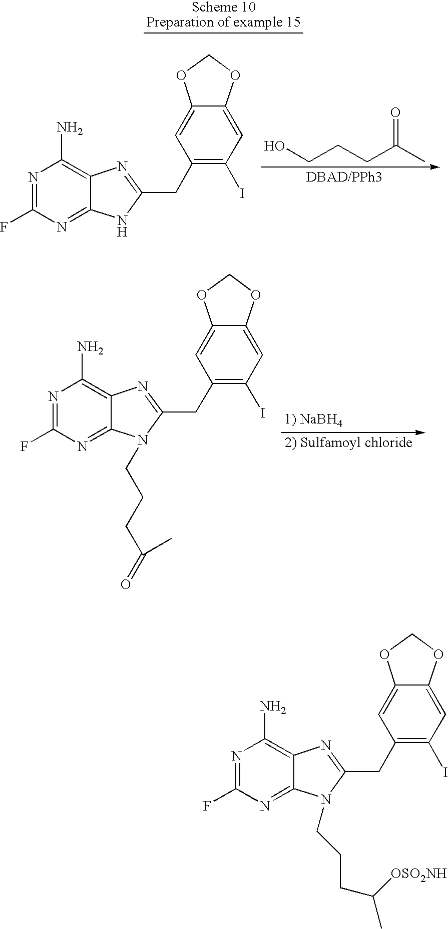

### EXAMPLE 15

### Sulfamic Acid 4-[6-amino-2-fluoro-8-(6-iodo-benzo[1,3]dioxol-5-ylmethyl)-purin-9-yl]-4-methyl-butyl ester

### 5-[6-Amino-2-fluoro-8-(6-iodo-benzo[1,3]dioxol-5-ylmethyl)-purin-9-yl]-pentan-2-one

A mixture of intermediate 5 (33 mg, 0.080 mmol), DBAD (36.8 mg, 0.16 mmol), triphenylphosphine (42 mg, 0.16 mmol) and 5-hydroxy-pentan-2-one (16.5 mg, 0.16 mmol) in DCM and toluene (2 mL, 1:5) was stirred overnight and concentrated in vacuo. The residue was purified by preparative TLC (eluted with toluene/EtOAc=3:1) to give the compound as white solid. (8 mg, 20% yield).

### 5-[6-Amino-2-fluoro-8-(6-iodo-benzo[1,3]dioxol-5-ylmethyl)-purin-9-yl]-pentan-2-ol

5-[6-Amino-2-fluoro-8-(6-iodo-benzo[1,3]dioxol-5-ylmethyl)-purin-9-yl]-pentan-2-one (8 mg, 0.0161 mmol) was dissolved in 1 mL of MeOH at 0° C. NaBH₄ (8 mg, 0.211 mmol) was added and the reaction mixture was stirred at 0° C. for 1 hour. The solvent was removed under reduced pressure and the residue was purified by preparative TLC (eluted with CHCl₃/MeOH=10:1) to give the compound as white solid (5 mg, 62% yield).

### Sulfamic Acid 4-[6-amino-2-fluoro-8-(6-iodo-benzo[1,3]dioxol-5-ylmethyl)-purin-9-yl]-4-methyl-butyl ester

A solution of 5-[6-Amino-2-fluoro-8-(6-iodo-benzo[1,3]dioxol-5-ylmethyl)-purin-9-yl]-pentan-2-ol (10 mg, 0.02 mmol), CaCO₃(20 mg, 0.20 mmol), and sulfamoyl chloride (20 mg, 0.173 mmol) in anhydrous DMF (0.5 mL) was stirred at room temperature for 2 h. The solvent was removed under reduced pressure, and the crude product was purified by chromatography on silica gel (eluted with EtOAc/hexanes/CHCl₃/i-PrOH=10:10:10:1) to give the title compound as a white solid.

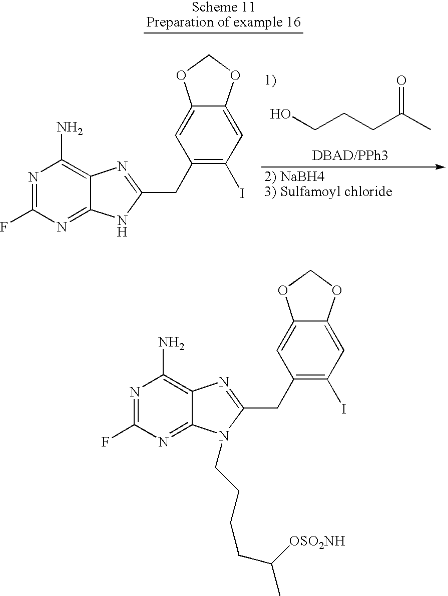

### EXAMPLE 16

### Sulfamic Acid 5-[6-amino-2-fluoro-8-(6-iodo-benzo[1,3]dioxol-5-ylmethyl)-purin-9-yl]-5-methyl-pentyl ester

### 6-[6-Amino-2-fluoro-8-(6-iodo-benzo[1,3]dioxol-5-ylmethyl)-purin-9-yl]-hexan-2-one

A mixture of intermediate 5 (35 mg, 0.085 mmol), DBAD (38.8 mg, 0.17 mmol), triphenylphosphine (44 mg, 0.17 mmol) and 6-hydroxy-hexan-2-one (20 mg, 0.17 mmol) in DCM and toluene (2 mL, 1:5) was stirred overnight and concentrated in vacuo. The residue was purified by preparative TLC (eluted with toluene/EtOAc=3:1) to give the compound as a white solid (3 mg, 6.9% yield).

### 6-[6-Amino-2-fluoro-8-(6-iodo-benzo[1,3]dioxol-5-ylmethyl)-purin-9-yl]-hexan-2-ol

6-[6-Amino-2-fluoro-8-(6-iodo-benzo[1,3]dioxol-5-ylmethyl)-purin-9-yl]-hexan-2-one (0.0161 mmol) was dissolved in 1 mL of MeOH at 0° C. NaBH₄ (8 mg, 0.211 mmol) was added and the reaction mixture was stirred at 0° C. for 1 hour. The solvent was removed under reduced pressure and the residue was purified by preparative TLC (eluted with CHCl₃/MeOH=10:1) to give the compound.

### Sulfamic Acid 5-[6-amino-2-fluoro-8-(6-iodo-benzo[1,3]dioxol-5-ylmethyl)-purin-9-yl]-5-methyl-pentyl ester

A solution of 6-[6-Amino-2-fluoro-8-(6-iodo-benzo[1,3]dioxol-5-ylmethyl)-purin-9-yl]-hexan-2-ol (10 mg, 0.02 mmol), CaCO₃ (20 mg, 0.20 mmol), and sulfamoyl chloride (20 mg, 0.173 mmol) in anhydrous DMF (0.5 mL) was stirred at room temperature for 2 h. The solvent was removed under reduced pressure, and the crude product was purified by chromatography on silica gel (eluted with EtOAc/hexanes/CHCl₃/i-PrOH=10:10:10:1) to give the title compound as a white solid.

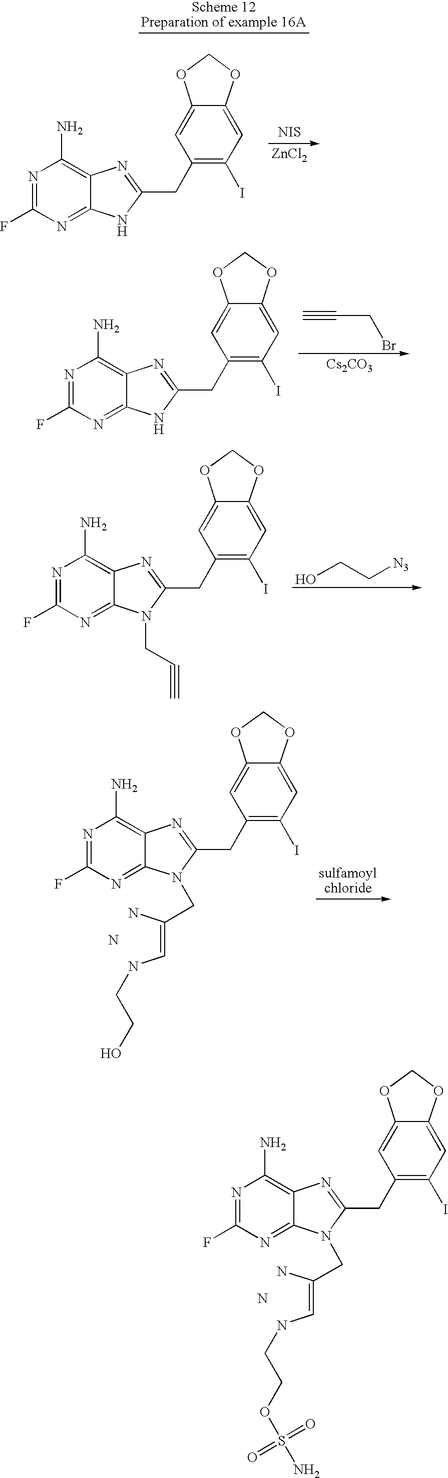

### EXAMPLE 16A

### Sulfamic Acid 2-(4-((6-amino-2-fluoro-8-((6-iodobenzo[d][1,3]dioxol-5-yl)methyl)-9H-purin-9-yl)methyl)-1H-1,2,3-triazol-1-yl)ethyl ester

2-Bromoethanol (8.5 mL, 120 mmol) was added to a solution of sodium azide (10.1 g, 156.0 mmol), and sodium hydroxide (480 mg, 12.0 mmol) in water (70 mL). The mixture was stirred at room temperature for two days, sodium sulfate (17.5 g) was added and after 10 mins the mixture was extracted with dichloromethane (3×50 mL). The combined extract were dried (sodium sulfate) and concentrated, and the residue, ˜10 g was used without further purification.

A solution of 8-(1,3-benzodioxol-5-ylmethyl)-2-fluoro-9H-purin-6-amine (1.6 g, 5.6 mmol), N-iodosuccinimide (5.1 g, 22.4 mmol), and zinc chloride (2.3 g, 16.8 mmol) in acetic acid (50 mL) was stirred at room temperature over night. Following solvent removal, water was added and the resulting precipitate was collected by filtration and purified by flash chromatography (ethyl acetate/chloroform at 40:60, followed by methanol/ethyl acetate at 10:90) to provide 1.5 g (66%) of 2-fluoro-8-[(6-iodo-1,3-benzodioxol-5-yl)methyl]-9H-purin-6-amine as a brown solid.

MS (ESI) m/z 414.1 (M+H).

A solution of 2-fluoro-8-[(6-iodo-1,3-benzodioxol-5-yl)methyl]-9H-purin-6-amine (500 mg, 1.21 mmol), cesium carbonate (395 mg, 1.21 mmol), and propargyl bromide (0.16 mL, 1.82 mmol) in N,N-dimethylformamide (4.0 mL) was stirred at room temperature for 6 h. Following solvent removal, 2-fluoro-8-[(6-iodo-1,3-benzodioxol-5-yl)methyl]-9-prop-2-yn-1-yl-9H-purin-6-amine (260 mg, 48%) was purified by flash chromatography (ethyl acetate/hexane at 25:75, followed by methanol/ethyl acetate at 5:95).

MS (ESI) m/z 452.1 (M+H).

A solution of 2-fluoro-8-[(6-iodo-1,3-benzodioxol-5-yl)methyl]-9-prop-2-yn-1-yl-9H-purin-6-amine (400 mg, 0.89 mmol), azidoethanol (1.0 g, 11.57 mmol), and copper iodide (339 mg, 1.78 mmol) was stirred in 1:1 solution of t-butanol and water at 110° C. for 1 h. After cooling, the mixture was filtered and purified flash chromatography (methanol/ethyl acetate at 7:93) to provide 320 mg (67%) of 2-[4-({6-amino-2-fluoro-8-[(6-iodo-1,3-benzodioxol-5-yl)methyl]-9H-purin-9-yl}methyl)-1H-1,2,3-triazol-1-yl]ethanol as a brown solid.

MS (ESI) m/z 539.2 (M+H).

A solution of 2-[4-({6-amino-2-fluoro-8-[(6-iodo-1,3-benzodioxol-5-yl}methyl]-9H-purin-9-yl]methyl)-1H-1,2,3-triazol-1-yl]ethanol) (200 mg, 0.37 mmol) and triethylamine (0.5 mL, 3.7 mmol) in N,N-dimethylacetamide (3 mL) was cooled to 0° C. To the mixture was added sulfamoyl chloride (381 mg, 3.33 mmol) and allowed to stir at room temperature for 1 h. Water was added slowly and the resulting solid was collected by filtration and purified by flash chromatography (methanol/ethyl acetate at 15:85) to provide 95 mg, (41%) of sulfamic acid 2-(4-((6-amino-2-fluoro-8-((6-iodobenzo[d][1,3]dioxol-5-yl)methyl)-9H-purin-9-yl)methyl)-1H-1,2,3-triazol-1-yl)ethyl ester as a white solid.

MS (ESI) m/z 618.2 (M+H).

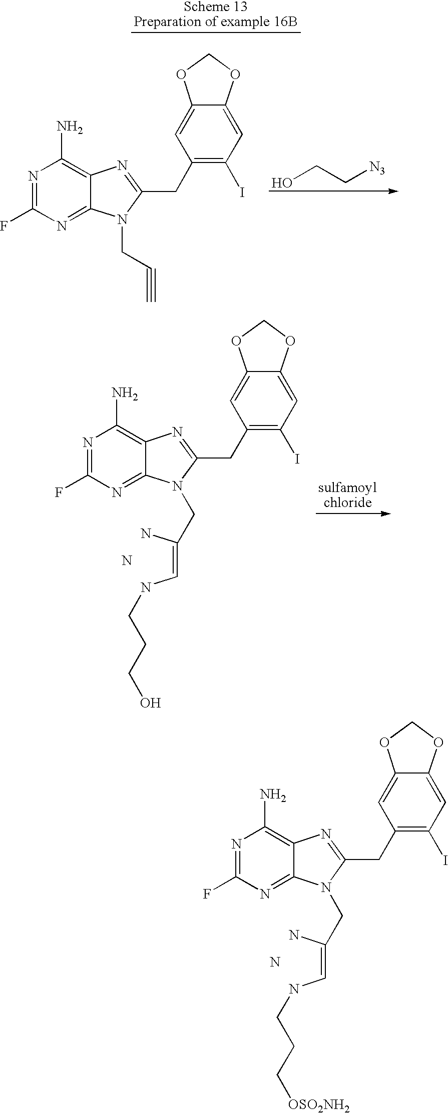

### EXAMPLE 16B

### Sulfamic Acid 3-[4-({6-amino-2-fluoro-8-[(6-iodo-1,3-benzodioxol-5-yl)methyl]-9H-purin-9-yl}methyl)-1H-1,2,3-triazol-1-yl]propyl ester

Starting from 2-bromopropanol (6 g, 43.17 mmol), and sodium azide (3.45 g, 56.12 mmol) and following the procedure for the synthesis of 2-azidoethanol, 8.3 g of 2-azidopropanol was obtained.

Starting from 300 mg (0.67 mmol) of 2-fluoro-8-[(6-iodo-1,3-benzodioxol-5-yl)methyl]-9-prop-2-yn-1-yl-9H-purin-6-amine, 2-azidopropanol (677 mg, 6.7 mmol) and copper iodide (255 mg, 1.34 mmol) and following the procedure for the synthesis of 2-[4-({6-amino-2-fluoro-8-[(6-iodo-1,3-benzodioxol-5-yl)methyl]-9H-purin-9-yl}methyl)-1H-1,2,3-triazol-1-yl]ethanol 63 mg (69%) of 3-[4-({6-amino-2-fluoro-8-[(6-iodo-1,3-benzodioxol-5-yl)methyl]-9H-purin-9-yl}methyl)-1H-1,2,3-triazol-1-yl]propan-1-ol was obtained.

MS (ESI) m/z 553.2 (M+H).

Starting from 3-[4-({6-amino-2-fluoro-8-[(6-iodo-1,3-benzodioxol-5-yl)methyl]-9H-purin-9-yl}methyl)-1H-1,2,3-triazol-1-yl]propan-1-ol (80 mg, 0.14 mmol), and sulfamoyl chloride (164 mg, 1.34 mmol) and following the procedure for the synthesis of 2-[4-({6-amino-2-fluoro-8-[(6-iodo-1,3-benzodioxol-5-yl)methyl]-9H-purin-9-yl}methyl)-1H-1,2,3-triazol-1-yl]ethyl sulfamate 63 mg (69%) of sulfamic acid 3-[4-({6-amino-2-fluoro-8-[(6-iodo-1,3-benzodioxol-5-yl)methyl]-9H-purin-9-yl}methyl)-1H-1,2,3-triazol-1-yl]propyl ester was obtained.

MS (ESI) m/z 632.2 (M+H).

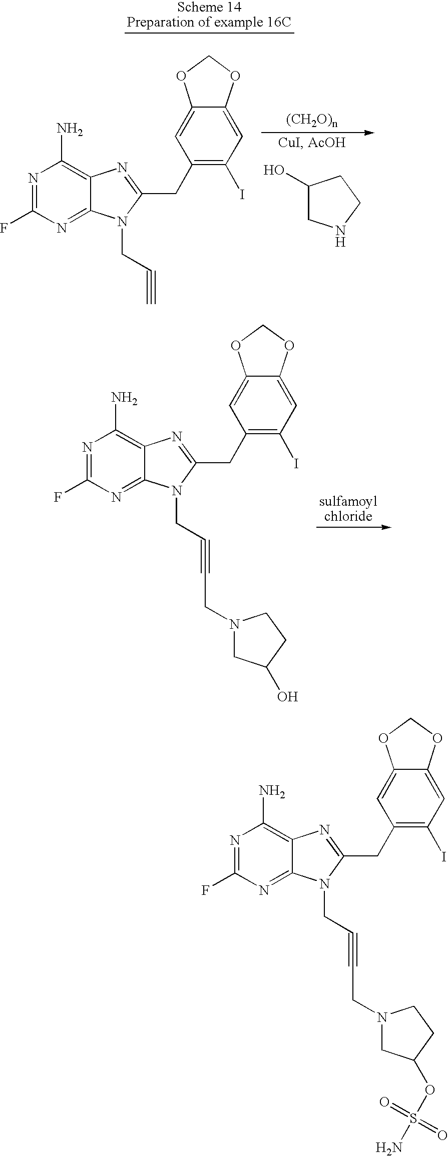

### EXAMPLE 16C

### Sulfamic Acid 1-(4-{6-amino-2-fluoro-8-[(6-iodo-1,3-benzodioxol-5-yl)methyl]-9H-purin-9-yl}but-2-yn-1-yl)pyrrolidin-3-ol ester

A solution of 2-fluoro-8-[(6-iodo-1,3-benzodioxol-5-yl)methyl]-9-prop-2-yn-1-yl-9H-purin-6-amine (200 mg, 0.44 mmol), 3-pyrrolidinol (383 mg, 4.4 mmol), paraformaldehyde (415 mg, 4.4 mmol), and copper iodide (419 mg, 2.2 mmol) was stirred in 10 mL solution of acetic acid and p-dioxane (1:15) and stirred at room temperature for 2 h. The mixture was filtered and washed with 1/1 methanol/dichloromethane and purified by flash chromatography (methanol/ethyl acetate at 20:80) to provide 130 mg (53%) of 1-(4-{6-amino-2-fluoro-8-[(6-iodo-1,3-benzodioxol-5-yl)methyl]-9H-purin-9-yl}but-2-yn-1-yl)pyrrolidin-3-ol as a brown solid.

MS (ESI) m/z 551.2 (M+H).

A solution of 1-(4-{6-amino-2-fluoro-8-[(6-iodo-1,3-benzodioxol-5-yl)methyl]-9H-purin-9-yl}but-2-yn-1-yl)pyrrolidin-3-ol (100 mg, 0.18 mmol), in N,N-dimethylacetamide (2 mL) was cooled to 0° C. To the mixture was added sulfamoyl chloride (209 mg, 1.18 mmol) and the reaction was allowed to stir at room temperature for 2 h. A solution of saturated sodium bicarbonate was added slowly and the resulting solid was collected by filtration and purified by flash chromatography (methanol/dichloromethane at 15:85) to provide 60 mg (53%) of sulfamic acid 1-(4-{6-amino-2-fluoro-8-[(6-iodo-1,3-benzodioxol-5-yl)methyl]-9H-purin-9-yl}but-2-yn-1-yl)pyrrolidin-3-ol ester as a white solid.

MS (ESI) m/z 630.2 (M+H).

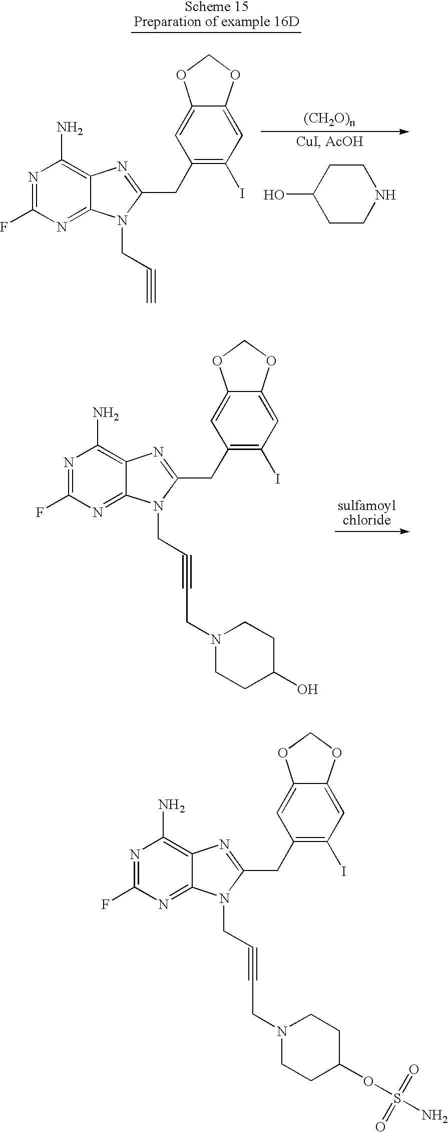

### EXAMPLE 16D

### Sulfamic Acid 1-(4-{6-amino-2-fluoro-8-[(6-iodo-1,3-benzodioxol-5-yl)methyl]-9H-purin-9-yl}but-2-yn-1-yl)piperidin-4-ol ester

Following the same procedure as in Example 16C, starting with 2-fluoro-8-[(6-iodo-1,3-benzodioxol-5-yl)methyl]-9-prop-2-yn-1-yl-9H-purin-6-amine and using 4-hydroxypiperidine instead of 3-pyrrolidinol, sulfamic acid 1-(4-{6-amino-2-fluoro-8-[(6-iodo-1,3-benzodioxol-5-yl)methyl]-9H-purin-9-yl}but-2-yn-1-yl)piperidin-4-ol ester was obtained as a white solid.

MS (ESI) m/z 644.2 (M+H).

C. Synthesis of Sulfamoyl-Containing Benzothiazolothio-/Pyridinothiazolothio-Derivatives

TABLE 1

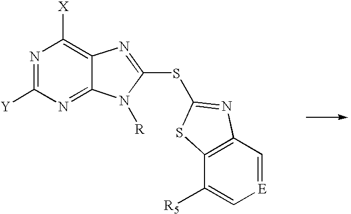

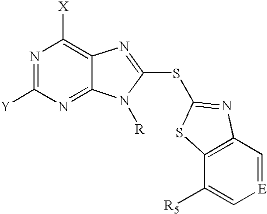

| Example No. A | B |
|---|---|
| 17 R = —CH₂CH₂OH | R′ = —CH₂CH₂OSO₂NR₁R₂ |
| 18 R = —CH₂CH₂CH₂OH | R′ = —CH₂CH₂CH₂OSO₂NR₁R₂ |
| 19 R = —CH₂CH₂CH₂CH₂OH | R′ = —CH₂CH₂CH₂CH₂OSO₂NR₁R₂ |
| 20 R = —CH₂CH₂NHC(CH₃)₃ | R′ = —CH₂CH₂N(SO₂NR₁R₂)C(CH₃)₃ |
| 21 R = —CH₂CH₂NHCH₂C(CH₃)₃ | R′ = —CH₂CH₂N(SO₂NR₁R₂)CH₂C(CH₃)₃ |
| 22 R = —CH₂CH₂NH-cyclopropane | R′ = —CH₂CH₂N(SO₂NR₁R₂)-cyclopropane |
| 23 R = —CH₂CH₂CH₂NHCH(CH₃)₂ | R′ = —CH₂CH₂CH₂N(SO₂NR₁R₂)CH(CH₃)₂ |
| 24 R = —CH₂CH₂CH₂NHC(CH₃)₃ | R′ = —CH₂CH₂CH₂N(SO₂NR₁R₂)C(CH₃)₃ |

Each of the compounds of structure A above (E can be carbon or nitrogen) can be prepared by methods known in the art [(see, for example, Zhang et al., J. Med. Chem., July, 2006, 49(17), 5352-5362) examples 17-24 employ X═NH₂, Y═H, R₅=Cl, E=C]. These can then be derivatized to form the corresponding sulfamoyl derivatives by reaction of the OH or NH groups on each of the R groups of compound A with sulfamoyl chloride as described elsewhere herein to form the corresponding structures of compound B with the indicated R′ group. Following known procedures described elsewhere herin also sulfamic acids [R′ containing —NR₁—SO₂OH as a substituent] are obtained. In such derivatives, R₁ has the meaning described elsewhere herein for Formula A.

Derivatizations may require protection and deprotection steps of functional groups not intended for derivatization as commonly conducted by someone skilled in the art.

D. More Examples of Synthesis of Sulfamoyl Derivatives

### EXAMPLE 24A

### Sulfamic Acid 3-(6-amino-8-(7-chlorobenzo[d]thiazol-2-ylthio)-9H-purin-9-yl)propyl ester

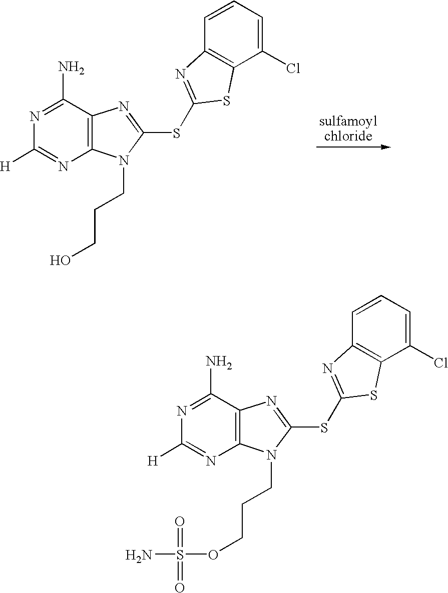 According to the procedure of Example 1, 3-(6-amino-8-(7-chlorobenzo[d]thiazol-2-ylthio)-9H-purin-9-yl)propan-1-ol, prepared as in J. Med. Chem. (2006), 49, 5352-5362, can be sulfamoylated with sulfamoyl chloride and calcium carbonate in DMF to provide sulfamic acid 3-(6-amino-8-(7-chlorobenzo[d]thiazol-2-ylthio)-9H-purin-9-yl)propyl ester.

### EXAMPLE 24B

### Sulfamic Acid 3-(6-amino-8-(7-chlorobenzo[d]thiazol-2-ylthio)-9H-purin-9-yl)butyl ester

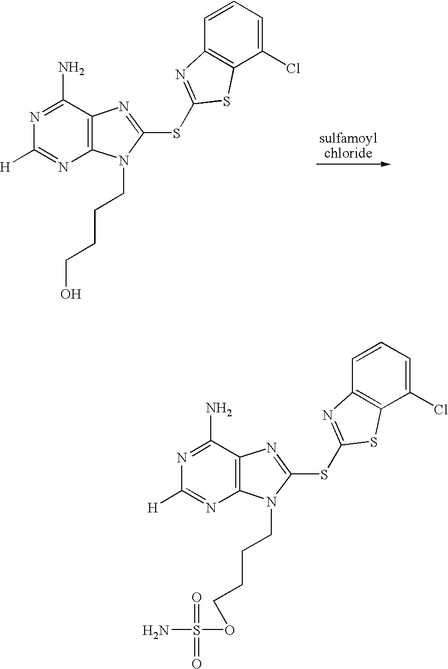 According to the procedure of Example 1, 3-(6-amino-8-(7-chlorobenzo[d]thiazol-2-ylthio)-9H-purin-9-yl)butan-1-ol, prepared as in J. Med. Chem. (2006), 49, 5352-5362, can be sulfamoylated with sulfamoyl chloride and calcium carbonate in DMF to provide sulfamic acid 3-(6-amino-8-(7-chlorobenzo[d]thiazol-2-ylthio)-9H-purin-9-yl)butyl ester.

### EXAMPLE 24C

### Sulfamic Acid 4-(7-amino-3H-[1,2,3]triazolo[4,5-d]pyrimidin-3-yl)butyl ester

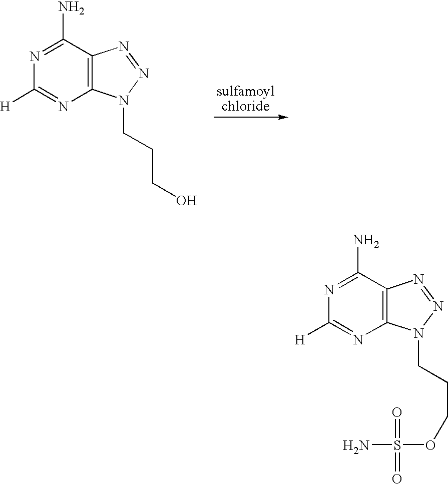 According to the procedure of Example 1, 4-(7-amino-3H-[1,2,3]triazolo[4,5-d]pyrimidin-3-yl)propan-1-ol, prepared as in J. Chem. Soc. (1960), 327-331, can be sulfamoylated with sulfamoyl chloride and calcium carbonate in DMF to provide sulfamic acid 4-(7-amino-3H-[1,2,3]triazolo[4,5-d]pyrimidin-3-yl)propyl ester.

### EXAMPLE 24D

### Sulfamic Acid 2-((4-amino-3-cyano-1H-pyrazolo[3,4-d]pyrimidin-1-yl)methoxy)ethyl ester

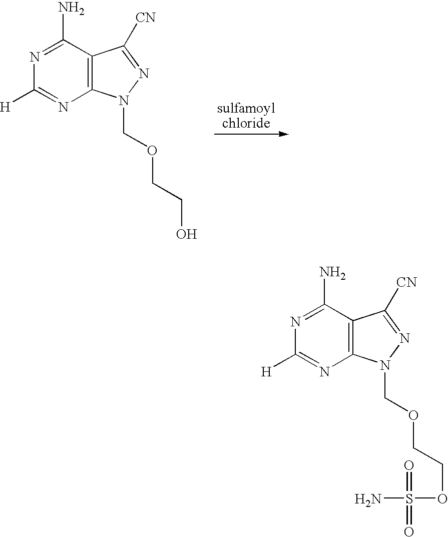 According to the procedure of Example 1, 4-amino-1-((2-hydroxyethoxy)methyl)-1H-pyrazolo[3,4-d]pyrimidine-3-carbonitrile, prepared as in J. Med. Chem. (1990), 33, 1980-1983, can be sulfamoylated with sulfamoyl chloride and calcium carbonate in DMF to provide sulfamic acid 2-((4-amino-3-cyano-1H-pyrazolo[3,4-d]pyrimidin-1-yl)methoxy)ethyl ester.

### EXAMPLE 24E

### Sulfamic Acid 2-((4-amino-5-carbamoyl-6-(methylthio)-7H-pyrrolo[2,3-d]pyrimidin-7-yl)methoxy)ethyl ester

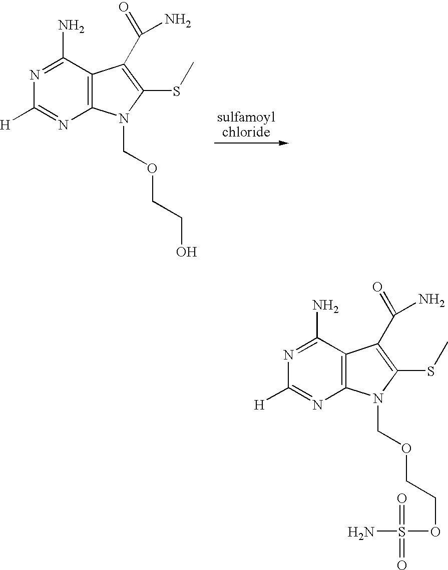 According to the procedure of Example 1, 4-amino-7-((2-hydroxyethoxy)methyl)-6-(methylthio)-7H-pyrrolo[2,3-d]pyrimidine-5-carboxamide, prepared as in J. Med. Chem. (1990), 33, 2162-2173, can be sulfamoylated with sulfamoyl chloride and calcium carbonate in DMF to provide sulfamic acid 2-((4-amino-5-carbamoyl-6-(methylthio)-7H-pyrrolo[2,3-d]pyrimidin-7-yl)methoxy)ethyl ester.

Derivatizations may require protection and deprotection steps of functional groups not intended for derivatization as commonly conducted by someone skilled in the art.

The invention also includes compounds of the following Formula:

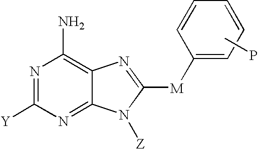

wherein M is selected from S, SO, SO₂, O, NH, CH₂ and CF₂. In other examples of this structure, the indicated substituents are as in Table 2.

In each of the compounds in Table 2 that already contain an alcohol or amine functional group at Z, the group at Z is readily converted to a sulfamoyl derivative by reaction of either the hydroxy or amino group on Z with sulfamoyl chloride to form the corresponding sulfamoyl derivative. The resulting sulfamoyl derivatives containing a —NSO₂N or —NSO₂OH or —OSO₂N structural element are compounds of the invention.

TABLE 2

| Example | | | | |
|---|---|---|---|---|
| No. | P | M | Z | Y |
| 25 | 2,5-dimethoxy | S | 3-hydroxypropyl | H |
| 26 | 2,5-dimethoxy | CH₂ | 3-ethylaminobutyl | H |
| 27 | 2,5-dimethoxy | CH₂ | hexan-6-ol | F |
| 28 | 2,5-dimethoxy | CH₂ | 3-hydroxypropyl | H |
| 29 | 2,5-dimethoxy | CH₂ | 3-hydroxypropyl | F |
| 30 | 2,5-dimethoxy | O | 3-hydroxypropyl | F |
| 31 | 2,5-dimethoxy | O | 3-hydroxypropyl | H |
| 32 | 2-iodo-5-methoxy | O | 3-hydroxypropyl | F |
| 33 | 2-iodo-5-methoxy | O | 3-hydroxypropyl | H |
| 34 | 2-iodo-5-methoxy | NH | 3-hydroxypropyl | F |
| 35 | 2-iodo-5-methoxy | NH | 3-hydroxypropyl | H |
| 36 | 2,5-dimethoxy | NH | 3-hydroxypropyl | F |
| 37 | 2,5-dimethoxy | NH | 3-hydroxypropyl | H |
| 38 | 2,5-dimethoxy | S | 3-hydroxypropyl | F |
| 39 | 2,5-dimethoxy | CH₂ | 3-hydroxypropyl | H |
| 40 | 2-iodo-5-methoxy | CH₂ | 3-hydroxypropyl | H |
| 41 | 2-iodo-5-methoxy | S | 3-hydroxypropyl | H |
| 42 | 2-iodo-5-methoxy | S | 3-hydroxypropyl | F |
| 43 | 2-iodo-5-methoxy | S | 3-hydroxypropyl | Cl |
| 44 | 2,5-dimethoxy | S | hexan-6-ol | H |
| 45 | 2,5-dimethoxy | S | hexan-6-ol | F |
| 46 | 2-iodo-5-methoxy | S | hexan-6-ol | H |
| 47 | 2-iodo-5-methoxy | S | hexan-6-ol | F |
| 48 | 2-iodo-5-methoxy | S | hexan-6-ol | H |
| 49 | 2-iodo-5-methoxy | CH₂ | hexan-6-ol | H |
| 50 | 2-iodo-5-methoxy | CH₂ | hexan-6-ol | F |
| 51 | 2,5-dimethoxy | S | pentan-5-ol | H |
| 52 | 2,5-dimethoxy | S | pentan-5-ol | F |
| 53 | 2-iodo-5-methoxy | S | pentan-5-ol | H |
| 54 | 2-iodo-5-methoxy | S | pentan-5-ol | F |
| 55 | 2-iodo-5-methoxy | S | pentan-5-ol | H |
| 56 | 2,5-dimethoxy | CH₂ | pentan-5-ol | H |
| 57 | 2,5-dimethoxy | CH₂ | pentan-5-ol | F |
| 58 | 2-iodo-5-methoxy | CH₂ | pentan-5-ol | H |
| 59 | 2-iodo-5-methoxy | CH₂ | pentan-5-ol | F |
| 60 | 2,5-dimethoxy | S | butan-4-ol | H |
| 61 | 2,5-dimethoxy | S | butan-4-ol | F |
| 62 | 2-iodo-5-methoxy | S | butan-4-ol | H |
| 63 | 2-iodo-5-methoxy | S | butan-4-ol | F |
| 64 | 2,5-dimethoxy | CH₂ | butan-4-ol | H |
| 65 | 2,5-dimethoxy | CH₂ | butan-4-ol | F |
| 66 | 2-iodo-5-methoxy | CH₂ | butan-4-ol | H |
| 67 | 2-iodo-5-methoxy | CH₂ | butan-4-ol | F |
| 68 | 2,5-dimethoxy | CH₂ | 4-ethylaminobutyl | F |
| 69 | 2,5-dimethoxy | S | 4-ethylaminobutyl | H |
| 70 | 2,5-dimethoxy | S | 4-ethylaminobutyl | F |
| 71 | 2-iodo-5-methoxy | CH₂ | 4-ethylaminobutyl | H |
| 72 | 2-iodo-5-methoxy | S | 4-ethylaminobutyl | H |
| 73 | 2-iodo-5-methoxy | S | 4-ethylaminobutyl | F |
| 74 | 2-iodo-5-methoxy | S | 4-ethylaminobutyl | Cl |
| 75 | 2,5-dimethoxy | CF₂ | 3-hydroxypropyl | H |
| 76 | 2,5-dimethoxy | CF₂ | 3-hydroxypropyl | F |
| 77 | 2,5-dimethoxy | CF₂ | 3-hydroxypropyl | Cl |
| 78 | 2,5-dimethoxy | CF₂ | 3-ethylaminobutyl | H |
| 79 | 2,5-dimethoxy | CF₂ | 3-ethylaminobutyl | F |
| 80 | 2,5-dimethoxy | CF₂ | 3-ethylaminobutyl | Cl |
| 81 | 2,5-dimethoxy | CF₂ | 4-ethylaminobutyl | H |
| 82 | 2,5-dimethoxy | CF₂ | 4-ethylaminobutyl | F |
| 83 | 2,5-dimethoxy | CF₂ | 4-ethylaminobutyl | Cl |
| 84 | 2,5-dimethoxy | CF₂ | hexan-6-ol | H |
| 85 | 2,5-dimethoxy | CF₂ | hexan-6-ol | F |
| 86 | 2,5-dimethoxy | CF₂ | hexan-6-ol | Cl |
| 87 | 2,5-dimethoxy | CF₂ | pentan-5-ol | H |
| 88 | 2,5-dimethoxy | CF₂ | pentan-5-ol | F |
| 89 | 2,5-dimethoxy | CF₂ | pentan-5-ol | Cl |
| 90 | 2,5-dimethoxy | CF₂ | butan-4-ol | H |
| 91 | 2,5-dimethoxy | CF₂ | butan-4-ol | F |
| 92 | 2,5-dimethoxy | CF₂ | butan-4-ol | Cl |
| 93 | 2-iodo-5-methoxy | CF₂ | 3-hydroxypropyl | H |
| 94 | 2-iodo-5-methoxy | CF₂ | 3-hydroxypropyl | F |
| 95 | 2-iodo-5-methoxy | CF₂ | 3-hydroxypropyl | Cl |
| 96 | 2-iodo-5-methoxy | CF₂ | 3-ethylaminobutyl | H |
| 97 | 2-iodo-5-methoxy | CF₂ | 3-ethylaminobutyl | F |
| 98 | 2-iodo-5-methoxy | CF₂ | 3-ethylaminobutyl | Cl |
| 99 | 2-iodo-5-methoxy | CF₂ | 4-ethylaminobutyl | H |
| 100 | 2-iodo-5-methoxy | CF₂ | 4-ethylaminobutyl | F |
| 101 | 2-iodo-5-methoxy | CF₂ | 4-ethylaminobutyl | Cl |
| 102 | 2-iodo-5-methoxy | CF₂ | hexan-6-ol | H |
| 103 | 2-iodo-5-methoxy | CF₂ | hexan-6-ol | F |
| 104 | 2-iodo-5-methoxy | CF₂ | hexan-6-ol | Cl |
| 105 | 2-iodo-5-methoxy | CF₂ | pentan-5-ol | H |
| 106 | 2-iodo-5-methoxy | CF₂ | pentan-5-ol | F |
| 107 | 2-iodo-5-methoxy | CF₂ | pentan-5-ol | Cl |
| 108 | 2-iodo-5-methoxy | CF₂ | butan-4-ol | H |
| 109 | 2-iodo-5-methoxy | CF₂ | butan-4-ol | F |
| 110 | 2-iodo-5-methoxy | CF₂ | butan-4-ol | Cl |

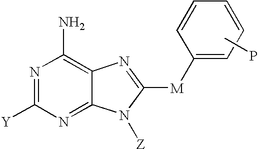

wherein M is selected from S, SO, SO₂, O, NH, CH₂ and CF₂. In other examples of this structure, the indicated substituents are as in Table 3.

Table 3 shows derivatives of the above formula that can be prepared as sulfamoyl derivatives by first derivatizing the group at Z so as to form a group reactive with sulfamoyl chloride. Reduction of, e.g., nitriles, ketones, esters, amides or hydrolysis of i.e. alkylhalides, esters results in the formation of hydroxy or amino group on Z. These derivatizations can also be conducted prior to the attachment of Z to the core structure of Formula A. The resulting sulfamoyl derivatives containing a —NSO₂N or —NSO₂OH or —OSO₂N structural element are compounds of the invention.

TABLE 3

| Example | | | | |
|---|---|---|---|---|
| No. | P | M | Z | Y |
| 111 | 2,5-dimethoxy | S | butyronitrile | H |
| 112 | 2,5-dimethoxy | S | 4-chlorobutyl | H |
| 113 | 2,5-dimethoxy | S | 4-acetoxybutyl | H |
| 114 | 2,5-dimethoxy | S | 5-bromopentyl | H |
| 115 | 2,5-dimethoxy | CH₂ | 4-chlorobutyl | H |
| 116 | 2,5-dimethoxy | CH₂ | 5-acetoxypentyl | H |
| 117 | 2,5-dimethoxy | CH₂ | 5-bromopentyl | H |
| 118 | 2,5-dimethoxy | CH₂ | 5-bromopentyl | F |
| 119 | 2,5-dimethoxy | CH₂ | 5-bromo-3-methyl-pentyl | H |
| 120 | 2,5-dimethoxy | CH₂ | 5-chloropentyl | H |
| 121 | 2,5-dimethoxy | CH₂ | butyronitrile | H |
| 122 | 2,5-dimethoxy | CH₂ | butyronitrile | F |
| 123 | 2,5-dimethoxy | O | butyronitrile | F |
| 124 | 2,5-dimethoxy | O | butyronitrile | H |
| 125 | 2-iodo-5-methoxy | O | butyronitrile | F |
| 126 | 2-iodo-5-methoxy | O | butyronitrile | H |
| 127 | 2-iodo-5-methoxy | NH | butyronitrile | F |
| 128 | 2-iodo-5-methoxy | NH | butyronitrile | H |
| 129 | 2,5-dimethoxy | NH | butyronitrile | F |
| 130 | 2,5-dimethoxy | NH | butyronitrile | H |
| 131 | 2,5-dimethoxy | S | butyronitrile | F |
| 132 | 2-iodo-5-methoxy | CH₂ | butyronitrile | H |
| 133 | 2-iodo-5-methoxy | S | butyronitrile | H |
| 134 | 2-iodo-5-methoxy | S | butyronitrile | Cl |
| 135 | 2,5-dimethoxy | CH₂ | 4-chlorobutyl | F |
| 136 | 2,5-dimethoxy | O | 4-chlorobutyl | F |
| 137 | 2,5-dimethoxy | O | 4-chlorobutyl | H |
| 138 | 2-iodo-5-methoxy | O | 4-chlorobutyl | F |
| 139 | 2-iodo-5-methoxy | O | 4-chlorobutyl | H |
| 140 | 2-iodo-5-methoxy | NH | 4-chlorobutyl | F |
| 141 | 2-iodo-5-methoxy | NH | 4-chlorobutyl | H |
| 142 | 2,5-dimethoxy | NH | 4-chlorobutyl | F |
| 143 | 2,5-dimethoxy | NH | 4-chlorobutyl | H |
| 144 | 2,5-dimethoxy | S | 4-chlorobutyl | F |
| 145 | 2-iodo-5-methoxy | CH₂ | 4-chlorobutyl | H |
| 146 | 2-iodo-5-methoxy | S | 4-chlorobutyl | F |
| 147 | 2-iodo-5-methoxy | S | 4-chlorobutyl | H |
| 148 | 2-iodo-5-methoxy | S | 4-chlorobutyl | Cl |
| 149 | 2,5-dimethoxy | CH₂ | 4-acetoxybutyl | F |
| 150 | 2,5-dimethoxy | O | 4-acetoxybutyl | F |
| 151 | 2,5-dimethoxy | O | 4-acetoxybutyl | H |
| 152 | 2-iodo-5-methoxy | O | 4-acetoxybutyl | F |
| 153 | 2-iodo-5-methoxy | O | 4-acetoxybutyl | H |
| 154 | 2-iodo-5-methoxy | NH | 4-acetoxybutyl | F |
| 155 | 2-iodo-5-methoxy | NH | 4-acetoxybutyl | H |
| 156 | 2,5-dimethoxy | NH | 4-acetoxybutyl | F |
| 157 | 2,5-dimethoxy | NH | 4-acetoxybutyl | H |
| 158 | 2,5-dimethoxy | S | 4-acetoxybutyl | F |
| 159 | 2-iodo-5-methoxy | S | 4-acetoxybutyl | F |
| 160 | 2-iodo-5-methoxy | S | 4-acetoxybutyl | Cl |
| 161 | 2,5-dimethoxy | O | 5-bromopentyl | F |
| 162 | 2,5-dimethoxy | O | 5-bromopentyl | H |
| 163 | 2-iodo-5-methoxy | O | 5-bromopentyl | F |
| 164 | 2-iodo-5-methoxy | O | 5-bromopentyl | H |
| 165 | 2-iodo-5-methoxy | NH | 5-bromopentyl | F |
| 166 | 2-iodo-5-methoxy | NH | 5-bromopentyl | H |
| 167 | 2,5-dimethoxy | NH | 5-bromopentyl | F |
| 168 | 2,5-dimethoxy | NH | 5-bromopentyl | H |
| 169 | 2,5-dimethoxy | S | 5-bromopentyl | F |
| 170 | 2-iodo-5-methoxy | S | 5-bromopentyl | H |
| 171 | 2-iodo-5-methoxy | S | 5-bromopentyl | F |
| 172 | 2-iodo-5-methoxy | S | 5-bromopentyl | Cl |
| 173 | 2-iodo-5-methoxy | CH₂ | 5-acetoxypentyl | H |
| 174 | 2-iodo-5-methoxy | S | 5-acetoxypentyl | H |

Tables 4 to 13 show structures of precursors of the indicated formula that are readily converted to sulfamoyl derivatives of the Z radical shown in the table. Literature descriptions for synthesis of these precursors are also indicated in the table. The resulting sulfamoyl derivatives containing a —NSO₂N or —NSO₂OH or —OSO₂N structural element are compounds of the invention.

TABLE 4

| | |
|---|---|
| | Formula II |
| 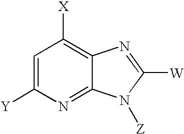 | |

| Example | | | | | |
|---|---|---|---|---|---|
| No. | X | Y | W | Z | Lit. |
| 175 | H | H | 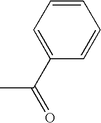 | 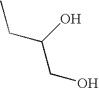 | J. Med. Chem., 1991, 34 (10), 2993-3006 |
| 176 | H | H | 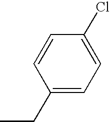 | 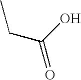 | J. Med. Chem., 1991, 34 (10), 2993-3006 |
| 177 | 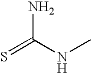 | H | 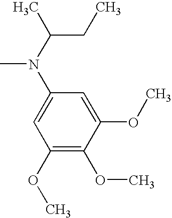 | 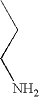 | US2006/173036 |
| 178 | H | H | 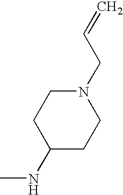 | 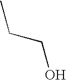 | US4943580 |
| 179 | H | H | 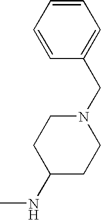 | 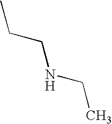 | US4943580 |

TABLE 5

| | |
|---|---|
| | Formula IV |
| 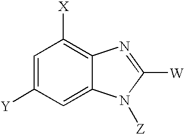 | |

| Example | | | | | |
|---|---|---|---|---|---|
| No. | X | Y | W | Z | Lit |
| 180 | H | H | 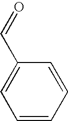 | 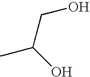 | J Heterocyclic Chem., 1983, 20, 1525-1532 |
| 181 | 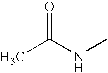 | H | 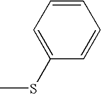 | 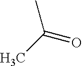 | Tetrahedron Letters, 2005, 46(27), 4613-4616 |

TABLE 6

| | |
|---|---|
| | Formula V |
| 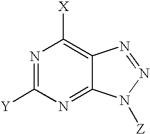 | |

| Example | | | | |
|---|---|---|---|---|
| No. | X | Y | Z | Lit |
| 182 | OH | H | 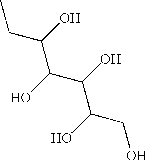 | J Chem. Soc., 1960, 5041-5047 |
| 183 | OH | H | 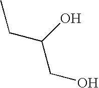 | Eur. J. Med. Chem., 1982, 17(6), 569- 571 |
| 184 | NH₂ | H | 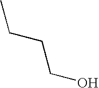 | J Chem. Soc., 1960, 5041-5047 |
| 185 | NH₂ | H | 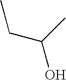 | Eur. J. Med. Chem., 1982, 17(6), 569- 571 |
| 186 | OH | NH₂ | 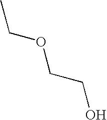 | US 4027025 |
| 187 | NMe₂ | H | 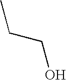 | J Chem. Soc., 1960, 327-330 |
| 188 | OH | OH | 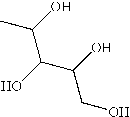 | J Chem. Soc., 1960, 5041-5047 |
| 189 | NH₂ | Cl | 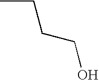 | J Heterocyclic Chem, 1990, 27(5), 1409- 1413 |
| 190 | NH₂ | Cl | 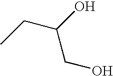 | J Heterocyclic Chem, 1990, 27(5), 1409- 1413 |
| 191 | NH₂ | NH₂ | 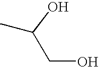 | J Med Chem, 1996, 39(20), 4073-4088 |

TABLE 7

| | |
|---|---|
| | Formula VI |
| 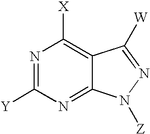 | |

| Example | | | | | |
|---|---|---|---|---|---|
| No. | X | Y | W | Z | Lit |
| 192 | NH₂ | H | 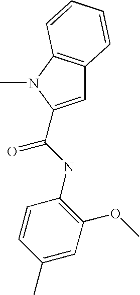 | 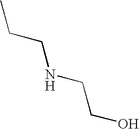 | US2004/6083 |
| 193 | NH2 | H | 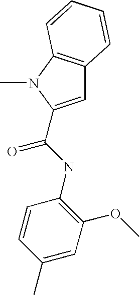 | 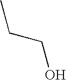 | US2004/6083 |
| 194 | NH₂ | NH₂ | 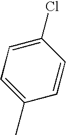 | 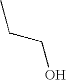 | J. Heterocyclic Chem., 1975, 12, 1199-1205 |
| 195 | NH₂ | H | 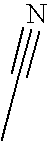 | 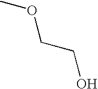 | J Med Chem, 1990, 33 (7), 1980- 1983 |
| 196 | NH₂ | H | CH₃ | 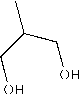 | Eur. J. Med. Chem. Chim. Ther., 1990, 25 (5), 419-424 |

TABLE 8

| | |
|---|---|
| | Formula VII |
| 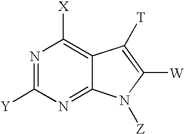 | |

| Example | | | | | | |
|---|---|---|---|---|---|---|
| No. | X | Y | T | W | Z | Lit |
| 197 | NH₂ | H | CH₃ | CH₃ | 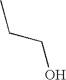 | Arch. Pharm. (Weinheim Ger.), 1975, 308, 252-258 |
| 198 | NH₂ | H | 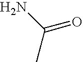 | 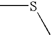 | 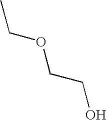 | J Med Chem, 1990, 33 (8), 2162-2173 |
| 199 | 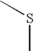 | 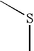 | CH₃ | Br | 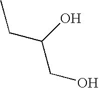 | Indian J. Chem. Sect. B, 1991, 30(5), 499-503 |
| 200 | NH₂ | H | 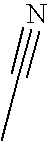 | Br | 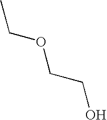 | Nucleosides & Nucleotides, 1999, 18(11-12), 2475-2498 |
| 201 | NH₂ | H | 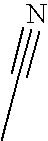 | Br | 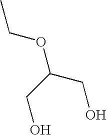 | Nucleosides & Nucleotides, 1999, 18(11-12), 2475-2498 |
| 202 | NH₂ | H | CH₃ | CH₃ | 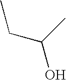 | J. Heterocycl. Chem., 1990, 27 (7), 2069-2075 |
| 203 | NH₂ | H | CH₃ | CH₃ | 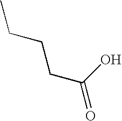 | J. Heterocycl. Chem., 1986, 23, 393-395 |

TABLE 9

| | |
|---|---|
| | Formula XXII |
| 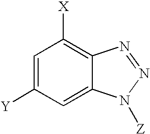 | |

| Example | | | | |
|---|---|---|---|---|
| No. | X | Y | Z | Lit |
| 204 | H | NH₂ | 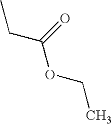 | JT Heterocycles, 2001, 55(6), 1133-1135 |
| 205 | H | Cl | 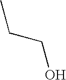 | Farmaco, 2001, 56 (4), 263-275 |
| 206 | H | OCH₃ | 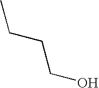 | Farmaco, 2001, 56 (4), 263-275 |
| 207 | H | OCH₃ | 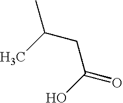 | Farmaco, 1994, 40 (11), 693-702 |
| 208 | H | NO₂ | 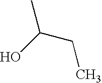 | J. Chem. Soc. Perkin Trans. 2,1990 (6), 921-924 |
| 209 | H | 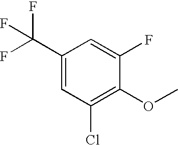 | 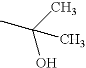 | US5324711 |
| 210 | H | 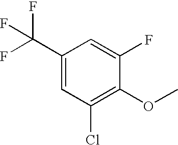 | 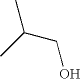 | EP355049 |
| 211 | H | 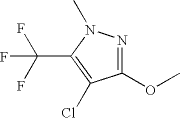 | 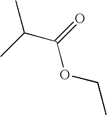 | US5190574 |
| 212 | H | 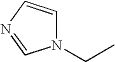 | 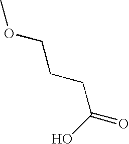 | US4943574 |

TABLE 10

| | |
|---|---|
| | Formula XXIII |
| 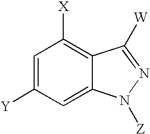 | |

| Example | | | | | |
|---|---|---|---|---|---|
| No. | X | Y | W | Z | Lit |
| 213 | H | OCH₃ | CH₃ | 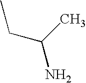 | US6956036 |
| 214 | H | OCH₃ | CH₃ | 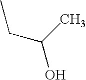 | US6956036 |
| 215 | H | OCH₃ | 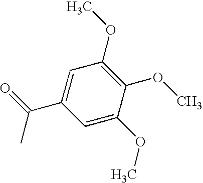 | 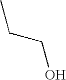 | WO2006/57946 |
| 216 | H | OCH₃ | 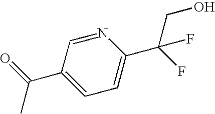 | 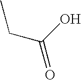 | WO2005/25568 |
| 217 | H | OCH₃ | 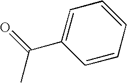 | 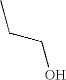 | US2004/97575 |
| 218 | H | CF₃ | 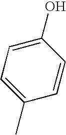 | 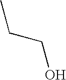 | WO2004/31159 |
| 219 | OCH₃ | CH₃ | 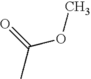 | 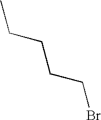 | Organic Letters, 2005, 7(12), 2449-2451 |

TABLE 11

| | |
|---|---|
| | Formula XXIV |
| 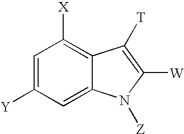 | |

| Example | | | | | | |
|---|---|---|---|---|---|---|
| No. | X | Y | T | W | Z | Lit |
| 220 | H | Cl | 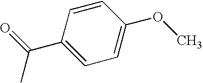 | CH₃ | 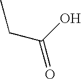 | Eur. J. Med. Chem. Chim. Ther., 1975, 10, 187-199 |
| 221 | H | Cl | 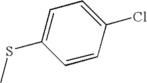 | CH₃ | 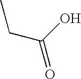 | PatentNumber WO2003/101961 |
| 222 | H | I | 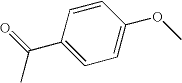 | CH₃ | 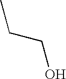 | J. Mass. Spectrom., 2004, 39 (6), 672-681 |
| 223 | H | I | 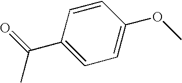 | CH₃ | 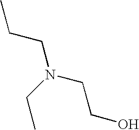 | J. Mass. Spectrom., 2004, 39 (6), 672-681 |

| | | | | | |
|---|---|---|---|---|---|
| 224 | H | F | —CH₂CH₂CH₂— | 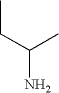 | US6706750 |
| 225 | Cl | Cl | —CH₂CH₂CH₂— | 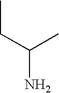 | US6706750 |

TABLE 12

| | |
|---|---|
| | Formula XXVI |
| 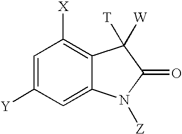 | |

| Example | | | | | | |
|---|---|---|---|---|---|---|
| No. | X | Y | T | W | Z | Lit |
| 226 | H | H | H | 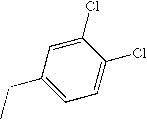 | 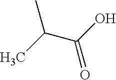 | Eur. J. Med. Chem. Chim. Ther., 1992, 27(8), 779-789 |
| 227 | H | F | H | 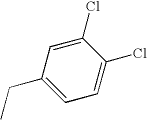 | 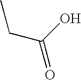 | Eur. J. Med. Chem. Chim. Ther., 1992, 27(8), 779-789 |
| 228 | H | OCH₃ | H | 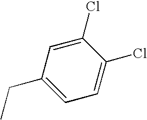 | 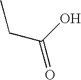 | Eur. J. Med. Chem. Chim. Ther., 1992, 27(8), 779-789 |

TABLE 13

| | |
|---|---|
| | Formula XXVII |
| 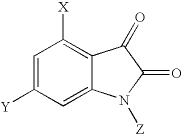 | |

| Example | | | | |
|---|---|---|---|---|
| No. | X | Y | Z | Lit |
| 229 | H | H | 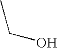 | Chim. Ther., 1973, 8, 447-450 |
| 230 | H | H | 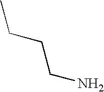 | NL 6507422, Chem Abst, 1966, 65, #2227 g |
| 231 | H | H | 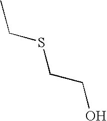 | Arch. Pharm. (Weinheim Ger.), 1973, 306, 684-692 |
| 232 | H | H | 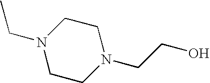 | NL 6507422, Chem Abst, 1966, 65, #2227 g |
| 233 | H | H | 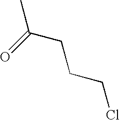 | J. Chem. Soc. Perkin Trans. 1, 1973, 998 |
| 234 | H | H | 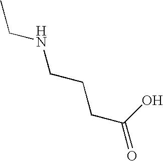 | US6203579 |

The present invention is directed to the clinical use of the heterocyclic ring with the general structure of Formula A: 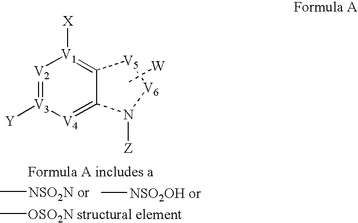
for example, purine derivatives of Formula I, such as adenine derivatives, such as those incorporating a sulfamoyl group, and related analogs of Formulas A1-A4, and their polymorphs, solvates, esters, tautomers, enantiomers, diastereomers, pharmaceutically acceptable salts and prodrugs thereof, for use in treatment or prevention of diseases that are HSP90-dependent. For example, a disorder such as inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, and malignant disease. The fibrogenetic disorders include but are not limited to scleroderma, polymyositis, systemic lupus, rheumatoid arthritis, liver cirrhosis, keloid formation, interstitial nephritis and pulmonary fibrosis.

The present invention features pharmaceutical compositions comprising the compounds at the experimental part and at Formulas A1 to A4, or a polymorph, solvate, ester, tautomer, pharmaceutically acceptable salt thereof, or prodrug thereof, of any of the preceding aspect and embodiments and one or more pharmaceutical excipients.

Those of ordinary skill in the art are familiar with formulation and administration techniques that can be employed with the compounds and methods of the invention, e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

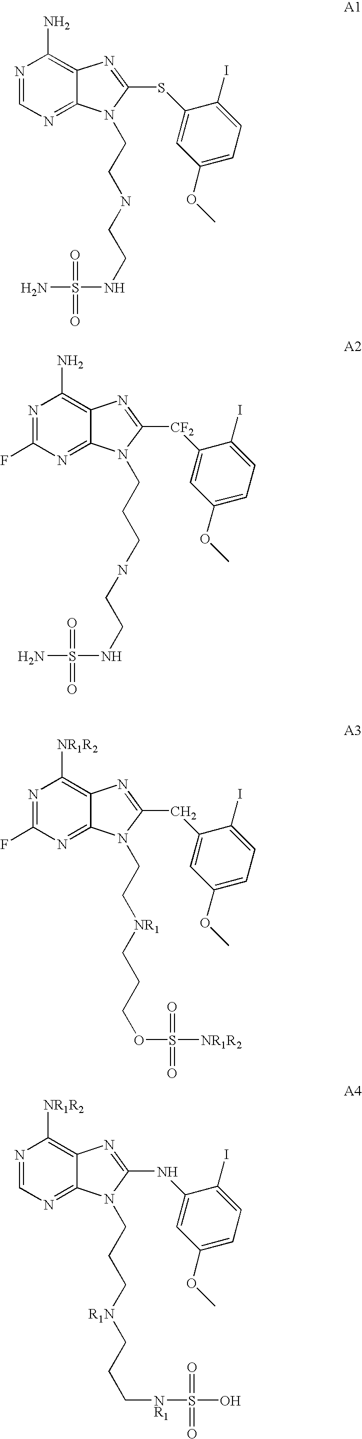

The compounds utilized in the methods of the instant invention may be administered either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

For example, the therapeutic or pharmaceutical compositions of the invention can be administered locally to the area in need of treatment. This may be achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue.

Still further, the compounds or compositions of the invention can be delivered in a vesicle, e.g., a liposome (see, for example, Langer, Science 1990, 249, 1527-1533; Treat et al., Liposomes in the Therapy of Infectious Disease and Cancer, Lopez-Bernstein and Fidler, Ed., Liss, N.Y., pp. 353-365, 1989).

The compounds and pharmaceutical compositions used in the methods of the present invention can also be delivered in a controlled release system. In one embodiment, a pump may be used (see, Sefton, 1987; CRC Crit. Ref. Biomed. Eng. 14:201; Buchwald et al. Surgery, 1980 88, 507; Saudek et al. N. Engl. J. Med. 1989, 321, (574). Additionally, a controlled release system can be placed in proximity of the therapeutic target. (See, Goodson, Medical Applications of Controlled Release, 1984, Vol. 2, pp. 115-138).

The pharmaceutical compositions used in the methods of the instant invention can also contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions, and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, cornstarch, or alginic acid; binding agents, for example starch, gelatin, polyvinyl-pyrrolidone or acacia, and lubricating agents, for example, magnesium stearate, stearic acid or talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The compounds and pharmaceutical compositions used in the methods of the instant invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening agents, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may, be first dissolved in a mixture of soybean oil and lecithin. The oil solution then introduced into a water and glycerol mixture and processed to form a microemulsion.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS.™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention used in the methods of the present invention may also be administered in the form of suppositories for rectal administration of the drug. These compositions can be prepared by mixing the inhibitors with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing a compound or composition of the invention can be used. As used herein, topical application can include mouth washes and gargles.

The compounds used in the methods of the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The methods, compounds and compositions of the instant invention may also be used in conjunction with other well known therapeutic agents that are selected for their particular usefulness against the condition that is being treated. For example, the instant compounds may be useful in combination with known anti-cancer and cytotoxic agents. Further, the instant methods and compounds may also be useful in combination with other inhibitors of parts of the signaling pathway that links cell surface growth factor receptors to nuclear signals initiating cellular proliferation.

The methods of the present invention may also be useful with other agents that inhibit angiogenesis and thereby inhibit the growth and invasiveness of tumor cells, including, but not limited to VEGF receptor inhibitors, including ribozymes and antisense targeted to VEGF receptors, angiostatin and endostatin.

Examples of antineoplastic agents that can be used in combination with the compounds and methods of the present invention include, in general, and as appropriate, alkylating agents, anti-metabolites, epidophyllotoxins, an antineoplastic enzyme, a topoisomerase inhibitor, procarbazine, mitoxantrone, platinum coordination complexes, biological response modifiers and growth inhibitors, hormonal/anti-hormonal therapeutic agents and haematopoietic growth factors. Exemplary classes of antineoplastic include the anthracyclines, vinca drugs, mitomycins, bleomycins, cytotoxic nucleosides, epothilones, discodermolide, pteridines, diynenes and podophyllotoxins. Particularly useful members of those classes include, for example, caminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

When a compound or composition of the invention is administered into a human subject, the daily dosage will normally be determined by the prescribing physician with the dosage generally varying according to the age, weight, and response of the individual patient, as well as the severity of the patient's symptoms.

In one application, a suitable amount of compound is administered to a mammal undergoing treatment for cancer, for example, breast cancer. Administration typically occurs in an amount of between about 0.01 mg/kg of body weight to about 100 mg/kg of body weight per day (administered in single or divided doses), more preferably at least about 0.1 mg/kg of body weight per day. A particular therapeutic dosage can include, e.g., from about 0.01 mg to about 1000 mg of compound, and preferably includes, e.g., from about 1 mg to about 1000 mg. The quantity of active compound in a unit dose of preparation may be varied or adjusted from about 0.1 mg to 1000 mg, preferably from about 1 mg to 300 mg, more preferably 10 mg to 200 mg, according to the particular application. The amount administered will vary depending on the particular IC₅₀ value of the compound used and the judgment of the attending clinician taking into consideration factors such as health, weight, and age. In combinational applications in which the compound is not the sole active ingredient, it may be possible to administer lesser amounts of compound and still have therapeutic or prophylactic effect.

Preferably, the pharmaceutical preparation is in unit dosage form. In such form, the preparation is subdivided into unit doses containing appropriate quantities of the active component, e.g., an effective amount to achieve the desired purpose.

The actual dosage employed may be varied depending upon the requirements of the patient and the severity of the condition being treated. Determination of the proper dosage for a particular situation is within the skill of the art. Generally, in determining the optimum dosage, a clinician may generally begin with smaller dosages that are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the compounds and compositions of the present invention used in the methods of the present invention, and if applicable other chemotherapeutic agents and/or radiation therapy, will be regulated according to the judgment of the attending clinician (physician) considering such factors as age, condition and size of the patient as well as severity of the disease being treated.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents.

Also, in general, the compounds of the invention need not be administered in the same pharmaceutical composition as another chemotherapeutic agent, and may, because of different physical and chemical characteristics, be administered by a different route. For example, the compounds/compositions may be administered orally to generate and maintain good blood levels thereof, while another chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times-of administration can be modified by the skilled clinician.

The particular choice of compound (and where appropriate, chemotherapeutic agent and/or radiation) will depend upon the diagnosis of the attending physicians and their judgment of the condition of the patient and the appropriate treatment protocol.

The compounds/compositions of the invention (and where appropriate chemotherapeutic agent and/or radiation) may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with the compound/composition.

In combinational applications and uses, the compound/composition and the chemotherapeutic agent and/or radiation need not be administered simultaneously or essentially simultaneously, and the initial order of administration of the compound/composition, and the chemotherapeutic agent and/or radiation, may not be important. Thus, the compounds/compositions of the invention may be administered first followed by the administration of the chemotherapeutic agent and/or radiation; or the chemotherapeutic agent and/or radiation may be administered first followed by the administration of the compounds/compositions of the invention. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the chemotherapeutic agent and/or radiation may be administered first, especially if it is a cytotoxic agent, and then the treatment continued with the administration of the compounds/compositions of the invention followed, where determined advantageous, by the administration of the chemotherapeutic agent and/or radiation, and so on until the treatment protocol is complete.

Thus, in accordance with experience and knowledge, the practicing physician can modify each protocol for the administration of a compound/composition for treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

The present invention also encompasses compositions comprising any one or more of the compounds of the invention in a pharmaceutically acceptable carrier. Compositions comprising any one or more of the compounds of Tables 1 to 13 are specific examples of such compositions. Such compositions may comprise additional agents, such as additional therapeutic agents, for example, anti-neoplastic agents, such as one or more of those selected from the group of a radioisotope, an antibody, a recombinant protein, Herceptin, taxol, taxane, gleevac, an alkylating agent, anti-metabolite, epidophyllotoxin, an antineoplastic enzyme, a topoisomerase inhibitor, procarbazine, mitoxantrone, a platinum coordination complex, a growth inhibitor, a hormonal therapeutic agent, an anti-hormonal therapeutic agent, a haematopoietic growth factor, an anthracycline drug, a vinca drug, a mitomycin, a bleomycin, a cytotoxic nucleoside, a tepothilone, a discodermolide, a pteridine drug, a diynesne, a podophyllotoxin, caminomycin, daunorubicin, an aminopterin, methotrexate, methopterin, dichloromethotrexate, mitomycin C, porfiromycin, 5-fluorouracil, 6-mercaptopurine, gemcitabine, cytosine arabinoside, podophyllotoxin, a podo-phyllotoxin, etoposide, etoposide phosphate, teniposide, melphalan, vinblastine, vincristine, leurosidine, vindesine, leurosine, paclitaxel, estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, a pyrodobenzoindole, an interferon and an interleukin.

The present invention also relates to a method of inhibiting an HSP90 protein comprising contacting a cell having or expressing an HSP90 protein with a compound according to the invention or any of the compositions of the invention. In one example, the cell is a mammalian cell, such as a human cell. In another such example, the method comprises some form of chemotherapy, which may be utilized in combination with other therapies, including radiation treatment and surgery. Such chemotherapy is for example directed against breast cancer cells and melanoma cells. Administration of any of the compounds, or compositions of said compounds, of the invention may be oral administration, or may be topical administration, or may be parenteral administration.

The present invention also relates to a method for treating an individual having a disease or condition selected from the group of inflammation, an infectious disease, an autoimmune disease, ischemia, a fibrogenetic disorder and nerve degeneration comprising administering to said individual a compound, or pharmaceutical composition, of the invention. Administration of any of the compounds, or compositions of said compounds, of the invention may be oral administration, or may be topical administration, or may be parenteral administration.

In specific examples of such treatment, the cancer over-expresses Her2 or steroid receptors. In other examples, the cancer is one that lacks functional retinoblastoma protein. In yet additional examples, the cancer is selected from the group consisting of breast cancer, small cell lung cancer, amyelocytic leukemia, vulvar cancer, non-small cell lung cancer, colon cancer, colorectal cancer, neuroblastoma and prostate cancer. The later is not an exclusive list of cancers that can be treated by the method.

Hsp90 CE Competitive Binding Assay:

Materials

a) Hsp90α Protein, 10.9 μM (Assay Designs/Stressgen)
The stock concentration of protein varies from batch-to-batch. The above is given as a typical example.

b) “Competitor”, fluorescein-labelled geldanamycin
The competitor was synthesized as described by Llauger-Bufi et al, Bioorg. Med. Chem. Lett., (2003), 13(22), 3975-3978.

c) Bovine serum albumin (BSA) (Sigma)

d) Dimethyl sulfoxide (DMSO) (Fluka)

e) CAPSO and Trizma Base (Sigma)

f) Dithiothreitol (DTT) (Sigma)

g) Magnesium chloride (MgCl₂) (Sigma)

h) Triton X-100 (Sigma)

i) Glacial acetic acid (JT Baker)

j) 17-AAG (Invivogen)

The electrophoresis running buffer (R_{B}) is comprised of 200 mM CAPSO, pH 9.2 (pH-adjusted with Trizma base). The sample buffer (SB) is comprised of 10 mM Trizma base, pH 8.0 (pH-adjusted with glacial acetic acid), 5 mM MgCl₂, 5 mM DTT, and BSA and Triton as noted.

Apparatus

Capillary electrophoresis was performed on Cetek Gemini Systems™ using laser-induced fluorescence detection. Fused-silica capillaries (50 μm I.D.×30 cm, approximately 10 cm length from end to detection window) were internally coated using Cetek's proprietary methods. LIF detection is performed using an argon laser for excitation (wavelength, 488 nm) and monitoring fluorescent emissions at 520 nm.

Methods

Hsp90α protein is diluted in SB containing 0.25 mg/ml BSA and 0.005% Triton to yield a “Target Cocktail” at an Hsp90 concentration of 42 nM. A “Competitor Cocktail” is prepared containing 220 nM Competitor in a solution of SB, 0.01 mg/ml BSA, and 0.02% Triton.

Each sample (i.e., fermentation extract, partially-purified fraction, or compound mixture, or pure compound) to be assayed is prediluted 1:20 or 1:40 in SB containing 0.02% Triton. 1 μl of each prediluted sample is then deposited into a 96-well microtiter plate and mixed with 8 μl of Target Cocktail. These samples can be stored at 4° C. until ready for assay.

When ready for analysis, the plates are transferred to the deck of a Gemini System. To each mixture of sample and target, 1 μl of Competitor Cocktail is added and mixed thoroughly. This reaction mixture is allowed to incubate for 8 minutes on the refrigerated deck of the Gemini System (temperature 8° C.). During incubation, the CE capillary is rinsed for 30 seconds with R_{B} containing 2 mg/ml BSA, and then 60 seconds with R_{B}. At the end of incubation, a small plug of the reaction mixture is then injected into the capillary at low pressure (0.5 psi) for 7 seconds. The loaded capillary is then dipped into reservoirs containing R_{B} and the sample is electrophoresed at 10 kV for 6 minutes at a temperature of 8° C.

The electrophoretic separation produces a graphical profile known as an “electropherogram,” which contains a peak representing the intact complex between Hsp90 and competitor, and a smaller series (typically, a triplet) of peaks representing unbound competitor. Ratiometric analysis of peak areas and/or heights of these two regions in the electropherogram is correlated to the amount of ligand displacement caused by an active component or compound, thus enabling the detection of “hits”. Dose-response experiments, which evaluate the degree of competitor displacement as a function of sample concentration, are subsequently run in triplicate to validate hits and to quantitate their potencies, which are expressed as IC₅₀ values.

To test the assay prior to operation, control samples are run to confirm proper performance. Negative controls are comprised of 0.5% DMSO, and positive controls are comprised of 0.1 μg/ml 17-AAG.

The ability of selected compounds of the invention to inhibit the binding of labeled geldanamycin to Hsp90 based on this assay is summarized in Table 14. IC₅₀ is defined as the concentration at which 50% inhibition is observed.

Luciferase Refolding Assay

Reagents:

A. Cancer cells are cultured according to ATCC guidelines. Cell lysates are prepared as follows to be used as source of heat shock proteins: Resuspend the washed cell pellet in 3˜10 packed cell volumes of Buffer A (10 mM HEPES pH 7.9, 1.5 mM MgCl₂, 10 mM KCl, 0.5 mM DTT, and 0.2 mM PMSF). Spin at 2000 rpm for 5 minutes in GS-6R (Beckman) centrifuge, 4° C. and resuspend cells in Buffer A up to a final of 3 packed cell volumes (i.e., add ˜2 volumes). Incubate on ice for 10 minutes. Transfer the cells to a Wheaton A Dounce homogenizer and lyse with 10-25 strokes. Stain an aliquot with Trypan Blue and check for >90% lysis under microscope. Spin the homogenized mixture at 3000 rpm for 20 minutes in GS-6R (Beckman) centrifuge, 4° C., to pellet nuclei. To the cytoplasmic fraction (cell lysate), add 1 mM ATP, 14.3 mM creatine phosphate, 71 μg/ml phosphokinase, 3 mM DTT, 113 mM KAc, and 0.7 mM MgAc₂. Aliquot into eppendorf tubes and freeze at −80° C.
B. Luciferase Stability Buffer (SB): 25 mM Tris-HCl, pH 7.8; 8 mM MgSO₄; 0.1 mM EDTA; 10 mg/ml BSA; 10% Glycerol; 0.25% Triton X-100. Make 2×SB and store at −20° C.
C. Cell Lysate (CL) Buffer: Buffer A containing 1 mM ATP; 14.3 mM creatine phosphate; 71 ug/ml phosphokinase; 3 mM DTT; 113 mM KAc; and 0.7 mM MgAc₂.
D. Luciferase: Promega E1701, 14.9 mg/ml=244 μM. Dilute 1:60 in 30 mM Tris-HCl, pH 7.4, 2 mM DTT, and 20% glycerol, resulting in a stock luciferase solution with concentration of 4 μM (250 ng/μl).
E. Luciferase substrate: Promega E1500.
   Procedure:
1) Mix luciferase solution 1:1 with 2× SB buffer. Incubate at 42° C. for 8 min to denature. After denaturation, leave the Luciferase on ice for 5 min to stabilize.
2) Assemble 100 μl refolding reactions: 68 μl CL buffer, 20 μl cell lysate or CL buffer as control, 2 μl Hsp90 inhibitor or DMSO (vehicle control), and 10 μl denatured Luciferase.
3) Incubate the above reactions at room temperature for 30 min.
4) Take 5 μl of a refolding reaction, mix with 30 μl luciferase substrate, and measure luminescence immediately with BIO-TEK Synergy HT (S=110).
   Data analysis: Luciferase refolding activity of a cell lysate sample with or without HSP90 inhibitor is represented as the net of luminescence reading from a cell lysate sample subtracting results from negative control (no cell lysate or heat shock proteins). Inhibition effect of a compound is calculated as the percentage of refolding activity relative to samples without any inhibitors in the cell lysate. EC₅₀ is determined as the compound concentration required for inhibiting 50% of luciferase refolding activity of a controlling cell lysate.

The ability of selected compounds of the invention to inhibit luciferase refolding by Hsp90 based on this assay is summarized in Table 14. IC₅₀ is defined as the concentration at which 50% inhibition is observed.

Hsp90 Client Protein Degradation Assay

SK-OV-3 cells were obtained from ATCC (Rockville, Md.) and maintained in McCoy's 5A Media with 10% Fetal Bovine Serum (ATCC) at 37° C. with 5% CO₂ in humidified incubators. Cells were plated at 2 ml per well at 100,000 cells/ml in six well dishes. Cells were incubated overnight at 37° C. and given test agent compound the following day in a titrated series. Cell incubation in the presence of compound proceeded overnight at 37° with 5% CO₂ in media in the presence of serum. The following day media was aspirated and cells washed on plate with 2 mls of cold D-PBS. 100 ul of 1× Laemli's buffer was added to each well and total protein obtained by scraping the cell pellet and removing to 1.5 ml Eppendorf tube on ice. Cells were further lysed and DNA was fragmented by sonication and cells debris pelleted at 14 K for 3 min at 4° C. 20 ul was loaded per well and fractionated on PAGE gels. Western analyses were performed using abs specifically for the Hsp90 client proteins HER-2 (Santa Cruz Biotech, Santa Cruz, Calif.) and c-raf-1 (Cell Signaling Technology, Danvers, Mass.). Loading per well was controlled by testing for g-actin on Western Blots (Cell Signaling Technology) simultaneous to testing for HER-2 or c-raf-1. Detection was via a secondary antibody conjugated to Horse radish peroxidase (HRP) and enhanced chemiluminescent detection from Supersignal West Pico Detection Kits from Pierce (Rockford, Ill.). Autoradiography was via Kodak X-OMAT film and developing via a Kodak M35 film processor.

The HER2 degradation ability of selected compounds of the invention based on this assay is summarized in Table 14. IC₅₀ is defined as the concentration at which 50% degradation of the HER2/Neu protein is observed.

Cancer-Derived Cell Line Growth Inhibition Assays

Human cancer cell lines were obtained from ATCC (Rockville, Md.) and maintained according to their media specifications at 37° C. in humidified incubators with or without 5% CO₂. Cells were plated at 0.1 ml/well of 50,000 cells per ml in 96 well plates. Monolayer grown cells were incubated overnight prior to the addition of compound while cells grown in suspension are given compound on the same day as plating. The test agent is titrated in duplicate for each compound and concentration tested. Cells are incubated in the presence of compound at 37° C. in humidified incubators for 3 days prior to addition of 20 ul of Cell Titer Blue (Promega, Madison, Wis.) which quantifies total cellular metabolic activity by conversion of resazurin to resorufin. Cell titer blue measures metabolic activity by quantifying the amount of biological activity present in a well using reduction of the dye resazurin as an end-point metric. Cells then are allowed to incubate at 37° C. for 2 hours and then analyzed using a Bio-TEK (Winooski, Vt.) Synergy HT 96/384 well plate reader set for 530 nm/590 nm, Ex/Em. Data analysis and depiction is conducted using EXCEL.

The cell growth inhibition of selected compounds of the invention based on this assay is summarized in Table 14. IC₅₀ is defined as the concentration at which 50% cell growth inhibition is observed.

TABLE 14

| | | | | Client | Cell |
|---|---|---|---|---|---|
| | | | | Protein | Growth |
| | | CE IC50 | Luciferase | IC50 | Inhibition |
| Example No. | Molecular Structure | (uM) | IC50 (uM) | (uM) | IC50 (uM) |
| 1 (R = SO2NH2) | 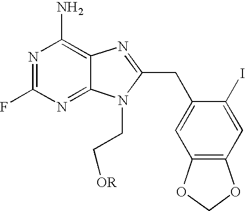 | <0.04 | <1.0 | <3.0 | <5.0 |
| 2 (R = SO2NH2) | 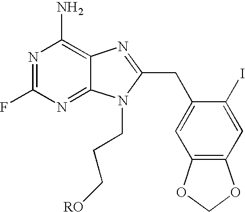 | <0.04 | <1.0 | <3.0 | <5.0 |
| 4 (R = SO2NH2) | 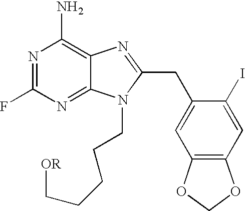 | <0.04 | <1.0 | <3.0 | <5.0 |
| 5 (R = SO2NH2) | 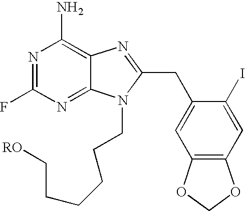 | <0.04 | <1.0 | <3.0 | <5.0 |
| 6 (R = SO2NH2) | 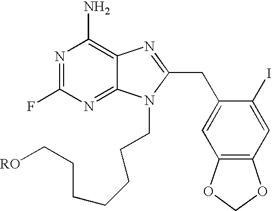 | <0.04 | <1.0 | <3.0 | <5.0 |
| 7 (R = SO2NH2) | 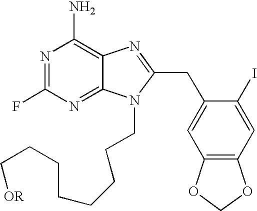 | <2.0 | <5.0 | <50.0 | <100.0 |
| 8 (R = SO2NH2) | 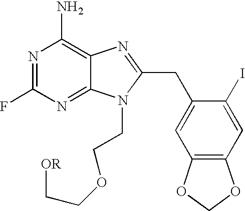 | <0.04 | <1.0 | <3.0 | <5.0 |
| 9 (R = SO2NH2) | 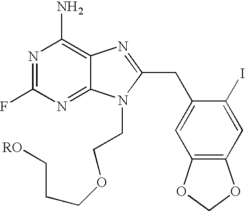 | <0.04 | <1.0 | <3.0 | <5.0 |
| 10 (R = SO2NH2) | 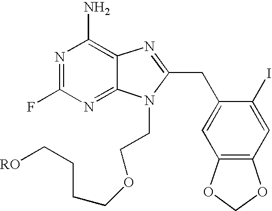 | <0.04 | <1.0 | <3.0 | <5.0 |
| 12 (R = SO2NH2) | 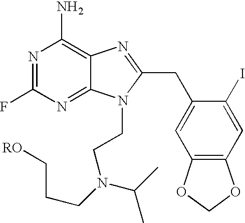 | <10.0 | <1.0 | <50.0 | <100.0 |
| Intermediate 5 | 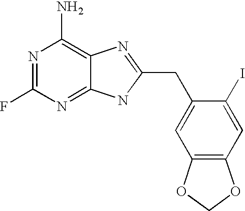 | <2.0 | <5.0 | <50.0 | <100.0 |

Compound 5 (R═SO₂NH₂) in Table 14 showed significantly more activity, 35 and 16 fold, than the corresponding alcohol (R═H) when tested in the CE Hsp90 assay and the client protein assay, respectively.
Hsp90alpha and BODIPY-Geldanamycin Fluorescent Polarization Binding Assay
Materials
HSP90alpha protein (Stressgen SPP-776) and BODIPY Labeled Geldanamycin (GA) was added to assay buffer stock (stored at 4 C): 20 mM Hepes pH 7.3, 50 mM KCl, 20 mM NaMoO4, 0.01% NP40
Working Assay buffer: assay buffer stock @RT with freshly added 2 mM DTT and 0.1 mg/ml
Bovine Gamma Globulin (Panvera P2045, 5 mg/ml)
Black 96well micro titer plates (Thermo #7205, vwr# 25227-304)
   Sample Set-up and Controls
Competitive Binding Samples: 5 nM GA and 30 nM Hsp90alpha and inhibitor
Control Samples: buffer only, BODIPY GA only, BODIPY GA plus Hsp90alpha, BODIPY GA plus
Hsp90alpha and unlabeled GA
   Protocol
   1. Prepare working assay buffer with Hsp90alpha protein at a final concentration of 30 nM per well in 100 ul final volume. Add 85 ul of this solution per well to 96 well black micro titer plate. (for 20 ml assay buffer add 400 ul 1 M DTT stk and 400 ul 5 mg/ml BGG stk) (1 mg/ml Hsp90 stock=˜11 uM protein; want 0.03 uM final in 100 ul, so adjust to 0.035 uM in 85 ul which is 1:314 fold dilution into assay buffer=64 ul protein plus 20 ml buffer to dispense 85 ul/well).
   2. Add 5 ul of inhibitors @ 20× final desired concentration. For 10 ug/ml start, the overall dilution from 1 mg/ml stocks is 1:100. (for 10 ug/ml start, prepare dmso cpd dilution plates with 1 mg/ml cpd stocks; serial dilute in dmso and titrate down 3-fold or 7 fold; use robot to transfer 10 ul from dmso cpd plate to 40 ul dmso intermediate plate (1:5 dilution, with intermediate plate at 200 ug/ml); use robot to then add 5 ul from the intermediate dmso plate to 85 ul in assay plate (1:20 dilution for 10 ug/ml final). Final assay condition will be 5% DMSO.
   3. Incubate protein and inhibitors for 10 min @ RT.
   4. Add BODIPY-GA in 10 ul @ 10× final desired concentration. (if 1× is 5 nM, 10× is 50 nM and dmso stock is 12 uM, so for 2.5 ml, use 10 ul of 12 uM GA plus 2.5 ml assay buffer and add 10 ul per well)
   5. Incubate at RT with gentle shaking for various time points (2 hr, 3 hr, etc.)
   6. Use Wallac Envision to read FP using protocol: JENs FP FITC Dual in room 4603. (parameters are: mirror module-FITC FP dual, excitation filter-FITC FP 480, emission filters-FITC FP P-pol 535 and FITC FP S-pol 535)
   Analysis of Results
   The Envision instrument automatically prepares a calculation based on the readings it obtains from the two measurements. The calculation expresses the results as mP units, millipolarization units, and the instrument calculation is as follows: mP value for FP measurement=1000*(S−G*P)/(S+G*P). The G value will be adjusted to <1 in order to move the baseline of the assay to zero. Samples wells containing BODIPY labeled geldanamycin and no hsp90 protein usually yield a mP value of −20 when G=1. By adjusting G to 0.96 or 0.97, the FP measurement calculation will result in a “0” reading for BODIPY GM without Hsp90. After this calculation, all data will be divided by the “untreated” average and multiplied by 100 to achieve % of control, or % polarization measurements. All data will have been transformed to a “0 to 100” scale. Therefore, the smaller the percent polarization value, the more active a compound is, because of it's ability to out—compete BODIPY GM and bind to Hsp90. IC50s will be determined manually from percent polarization values plotted in Microsoft Excel.
   The ability of selected compounds of the invention to inhibit the binding of labeled geldanamycin to Hsp90 based on this assay is summarized in Table 14A. IC₅₀ is defined as the concentration at which 50% inhibition is observed.
   REFERENCE COMPOUNDS: Unlabeled Geldanamycin is used as a positive control for competition with BODIPY labeled Geldanamycin for binding to the Hsp90 protein.

REFERENCES: Assay was based on the paper “Development of a Fluorescent Polarization Assay for the Molecular Chaperone Hsp90” Kim et al., 2004 Soc. For Biomolecular Screening.

TABLE 14A

| | | Binding |
|---|---|---|
| Example No. | Molecular Structure | IC₅₀ (uM) |
| 16A | 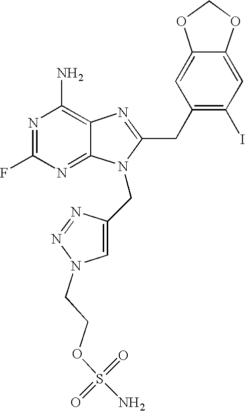 | 0.18 |
| 16B | 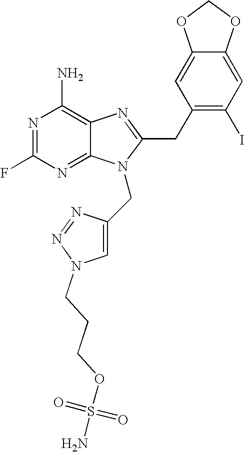 | 0.39 |
| 160 | 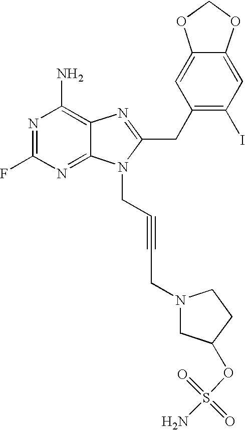 | 0.14 |

What is claimed is:

1. A compound of Formula I: 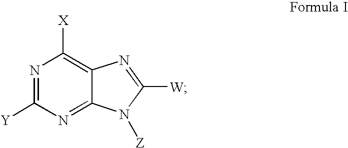
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

2. The compound of claim 1, wherein X is —NH₂.

3. The compound of claim 1, wherein Y is F or H.

4. The compound of claim 1, wherein W is: 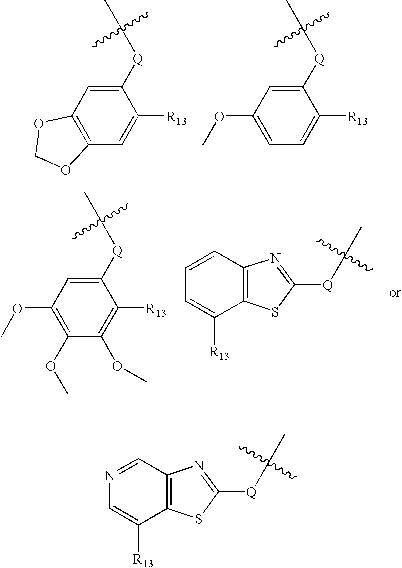
wherein Q is selected from —CR₁R₂—, carbonyl, difluoromethylene, —NR₁—, —O—, —S—, —SO—, and —SO₂—; and
R₁₃ is H, F, Cl, Br, I, OR₁, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C}.

5. The compound of claim 4, wherein Q is CH₂, C═O or CF₂.

6. The compound of claim 4, wherein Q is O or S.

7. The compound of claim 4, wherein R₁₃ is I.

8. The compound of claim 1, wherein said at least one of W, X, Y, or Z is —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C}.

9. The compound of claim 1, wherein Z is —R_{A}OSO₂N(R_{C})₂, or —R_{A}N(R_{C})OSO₂R_{C}.

10. The compound of claim 1, wherein Z is -heteroalkyl-OSO₂NH₂ or —C₁₋₆ alkyl-OSO₂NH₂.

11. The compound of claim 1, wherein W is 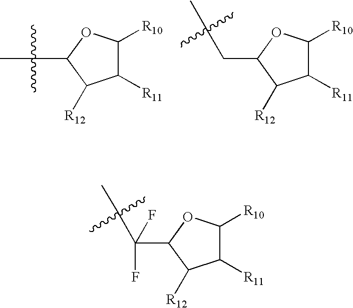
wherein
R₁₀, R₁₁ and R₁₂ are the same or different and each is H, SR_{C}, SOR_{C}, SO₂R₅ OR_{C}, COOR_{C}, CON(R_{C})₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₅R₆, —OSO₂N(R_{C})₂, —N(R_{C})SO₂OH or —N(R_{C}) SO₂N(R_{C})₂,
and wherein carbons substituted with R₁₁ and R₁₂ may be connected by single or double bond.

12. The compound of claim 1, wherein W is: 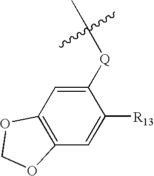
wherein,
R₁₃ is F, Cl, Br or I.

13. The compound of claim 12, wherein R₁₃ is I.

14. The compound of claim 1, wherein Z is —(CH₂)ₚL(CH₂)_{q}OSO₂NH₂;
L is —O—, —S—, N(R_{C}) or triazinyl; and
p and q are independently 1, 2, 3 or 4.

15. The compound of claim 1, selected from the group consisting of:
Sulfamic Acid 2-[6-amino-2-fluoro-8-(6-iodo-benzo[1,3]dioxol-5-ylmethyl)-purin-9-yl]-ethyl ester;
Sulfamic Acid 3-[6-Amino-2-fluoro-8-(6-iodo-benzo[1,3]dioxol-5-ylmethyl)-purin-9-yl]-propyl ester;
Sulfamic Acid 4-[6-amino-2-fluoro-8-(6-iodo-benzo[1,3]dioxol-5-ylmethyl)-purin-9-yl]-butyl ester;
Sulfamic Acid 5-[6-amino-2-fluoro-8-(6-iodo-benzo[1,3]dioxol-5-ylmethyl)-purin-9-yl]-pentyl ester;
Sulfamic Acid 6-[6-amino-2-fluoro-8-(6-iodo-benzo[1,3]dioxol-5-ylmethyl)-purin-9-yl]-hexyl ester;
Sulfamic Acid 7-[6-amino-2-fluoro-8-(6-iodo-benzo[1,3]dioxol-5-ylmethyl)-purin-9-yl]-heptyl ester;
Sulfamic Acid 8-[6-amino-2-fluoro-8-(6-iodo-benzo[1,3]dioxol-5-ylmethyl)-purin-9-yl]-octyl ester;
Sulfamic Acid 2-{2-[6-amino-2-fluoro-8-(6-iodo-benzo[1,3]dioxol-5-ylmethyl)-purin-9-yl]-ethoxy}-ethyl ester;
Sulfamic Acid 3-{2-[6-amino-2-fluoro-8-(6-iodo-benzo[1,3]dioxol-5-ylmethyl)-purin-9-yl]-ethoxy}-propyl ester;
Sulfamic Acid 4-{2-[6-amino-2-fluoro-8-(6-iodo-benzo[1,3]dioxol-5-ylmethyl)-purin-9-yl]-ethoxy}-butyl ester;
Sulfamic Acid 2-({2-[6-amino-2-fluoro-8-(6-iodo-benzo[1,3]dioxol-5-ylmethyl)-purin-9-yl]-ethyl}-isopropyl-amino)-ethyl ester;
Sulfamic Acid 3-({2-[6-amino-2-fluoro-8-(6-iodo-benzo[1,3]dioxol-5-ylmethyl)-purin-9-yl]-ethyl}-isopropyl-amino)-propyl ester;
Sulfamic Acid 4-({2-[6-amino-2-fluoro-8-(6-iodo-benzo[1,3]dioxol-5-ylmethyl)-purin-9-yl]-ethyl}-isopropyl-amino)-butyl ester;
Sulfamic Acid 3-[6-amino-2-fluoro-8-(6-iodo-benzo[1,3]dioxol-5-ylmethyl)-purin-9-yl]-3-methyl-propyl ester;
Sulfamic Acid 4-[6-amino-2-fluoro-8-(6-iodo-benzo[1,3]dioxol-5-ylmethyl)-purin-9-yl]-4-methyl-butyl ester;
Sulfamic Acid 5-[6-amino-2-fluoro-8-(6-iodo-benzo[1,3]dioxol-5-ylmethyl)-purin-9-yl]-5-methyl-pentyl ester;
Sulfamic Acid 2-(4-((6-amino-2-fluoro-8-((6-iodobenzo[d][1,3]dioxol-5-yl)methyl)-9H-purin-9-yl)methyl)-1H-1,2,3-triazol-1-yl)ethyl ester;
Sulfamic Acid 3-[4-({6-amino-2-fluoro-8-[(6-iodo-1,3-benzodioxol-5-yl)methyl]-9H-purin-9-yl}methyl)-1H-1,2,3-triazol-1-yl]propyl ester;
Sulfamic Acid 1-(4-{6-amino-2-fluoro-8-[(6-iodo-1,3-benzodioxol-5-yl)methyl]-9H-purin-9-yl}but-2-yn-1-yl)pyrrolidin-3-ol ester; and
Sulfamic Acid 1-(4-{6-amino-2-fluoro-8-[(6-iodo-1,3-benzodioxol-5-yl)methyl]-9H-purin-9-yl}but-2-yn-1-yl)piperidin-4-ol ester;
or a pharmaceutically acceptable salt thereof.

16. A composition comprising a compound of Formula I: 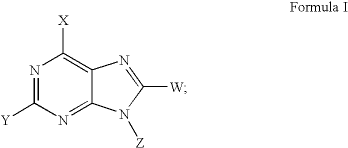
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W
and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}C, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof;
and a pharmaceutically acceptable carrier.

17. The composition of claim 16, wherein the pharmaceutically acceptable carrier is selected from the group consisting of lactose, glucose sucrose, corn starch, potato starch, sodium carboxymethyl cellulose, ethyl cellulose acetate, powdered tragacanth, malt, gelatin, talc, cocoa butter, peanut oil, cottonseed oil, safflower oil, sesame oil, olive oil, corn oil, soybean oil, polyethylene glycol, propylene glycol, ethyl oleate, ethyl laurate, agar, magnesium hydroxide, aluminum hydroxide, alginic acid, pyrogen-free water, isotonic saline, Ringer's solution, ethyl alcohol, a phosphate buffer solution, sodium lauryl sulfate, magnesium stearate, a coloring agent, a releasing agent, a coating agent, a sweetening agent, a flavoring agent, a perfuming agent, a preservative, and an antioxidant.

18. A method for inhibiting Hsp90 in a cell, comprising:
contacting the cell with a compound of Formula I: 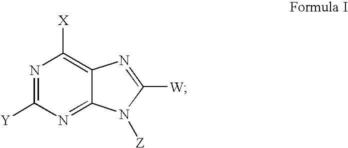
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

19. The method of claim 18, wherein the cell exhibits abnormal expression or activity of Hsp90.

20. The method of claim 19, wherein the cell is in vivo.

21. A method for treating an individual having cancer comprising administering to said individual a compound of Formula I: 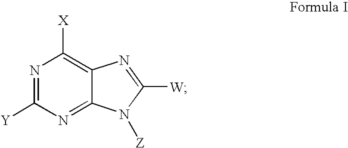
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

22. The method of claim 21, wherein the treatment of cancer further comprises administering another agent, wherein the other agent is an anti-neoplastic agent.

23. A method according to claim 21, wherein the anti-neoplastic agent is selected from the group of a radioisotope, an antibody, a recombinant protein, traztuzumab, taxol, taxane, gefitinib, imatinib, erlotinib, PTK-787, EKB-569, an alkylating agent, anti-metabolite, epidophyllotoxin, an antineoplastic enzyme, a topoisomerase inhibitor, procarbazine, mitoxantrone, a platinum coordination complex, a growth inhibitor, a hormonal therapeutic agent, an anti-hormonal therapeutic agent, a haematopoietic growth factor, an anthracycline drug, a vinca drug, a mitomycin, a bleomycin, a cytotoxic nucleoside, a tepothilone, a discodermolide, a pteridine drug, a diynesne, a podophyllotoxin, caminomycin, daunorubicin, an aminopterin, methotrexate, methopterin, dichloromethotrexate, mitomycin C, porfiromycin, 5-fluorouracil, 6-mercaptopurine, gemcitabine, cytosine arabinoside, podophyllotoxin, a podo-phyllotoxin, etoposide, etoposide phosphate, teniposide, melphalan, vinblastine, vincristine, leurosidine, vindesine, leurosine, paclitaxel, estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, a pyrodobenzoindole, an interferon and an interleukin.

24. The method of claim 21, wherein the cancer is selected from the group consisting of breast cancer, small cell lung cancer, amyelocytic leukemia, vulvar cancer, non-small cell lung cancer, colon cancer, colorectal cancer, neuroblastoma, myeloma and prostate cancer.

25. A compound of Formula II: 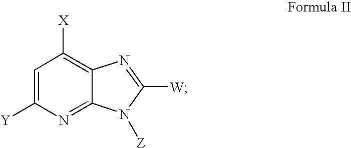
wherein
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, —NR₁, R₂—OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

26. A compound of Formula III: 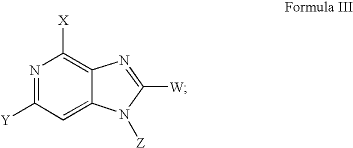
wherein
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, —NR₁, R₂—OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

27. A compound of Formula IV: 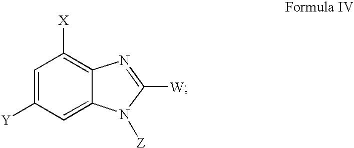
wherein
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, —NR₁, R₂—OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

28. A compound of Formula V 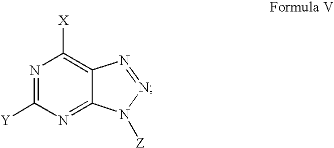
wherein
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

29. A compound of Formula VI: 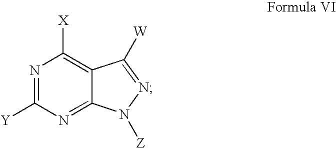
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

30. A compound of Formula VII: 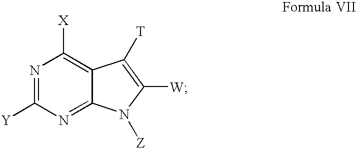
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

31. A compound of Formula VIII: 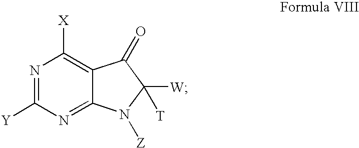
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

32. A compound of Formula IX: 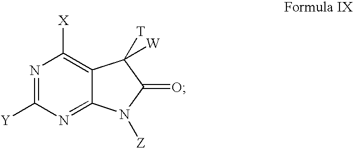
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

33. A compound of Formula X: 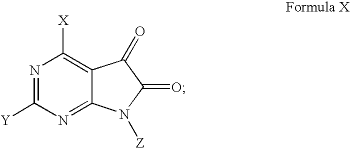
wherein,
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

34. A compound of Formula XI: 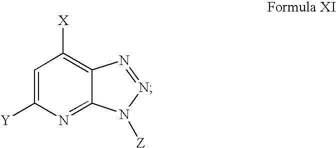
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

35. A compound of Formula XII: 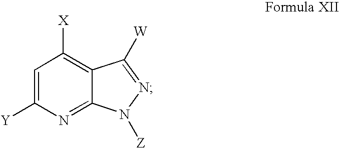
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

36. A compound of Formula XIII: 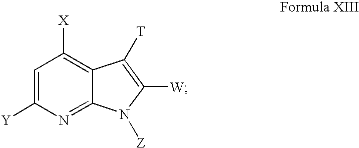
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

37. A compound of Formula XIV: 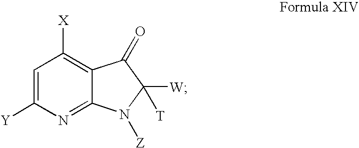
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

38. A compound of Formula XV: 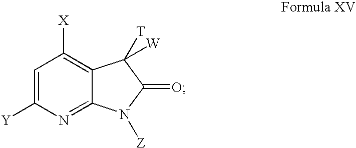
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

39. A compound of Formula XVI: 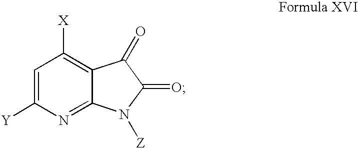
wherein,
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

40. A compound of Formula XVII: 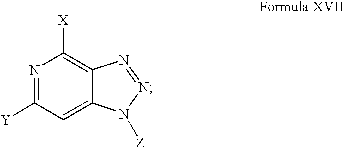
wherein,
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

41. A compound of Formula XVIII: 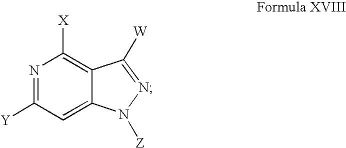
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

42. A compound of Formula XIX: 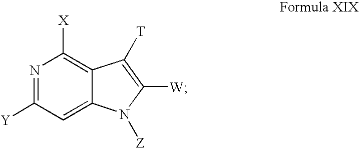
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

43. A compound of Formula XX: 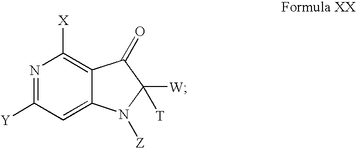
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

44. A compound of Formula XXI: 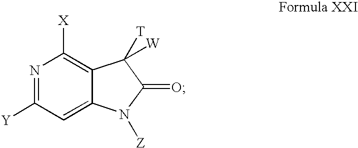
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

45. A compound of Formula XXII: 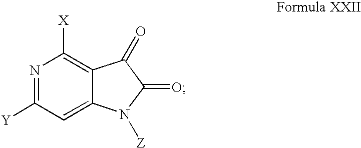
wherein,
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

46. A compound of Formula XXIIII: 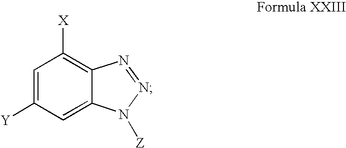
wherein,
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

47. A compound of Formula XXIV: 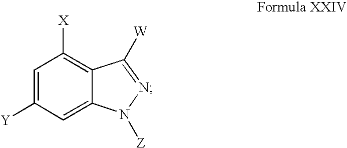
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

48. A compound of Formula XXV: 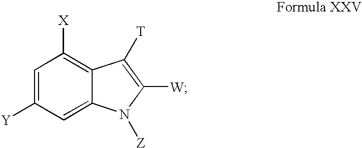
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

49. A compound of Formula XXVI: 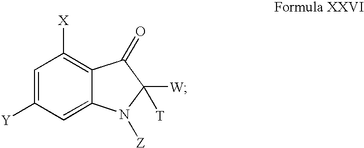
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

50. A compound of Formula XXVII: 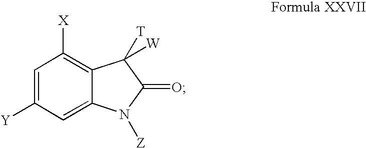
wherein,
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, NR₁R₂, —OSO₂NR₁R₂, —N(R₁) SO₂OH or —N(R₁)SO₂NR₁R₂, —OSO₂N(R_{C})₂, —N(R_{C})SO₂OH and —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
T is H, F, Cl, Br, I, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B}, or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of T, W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

51. A compound of Formula XXVIII: 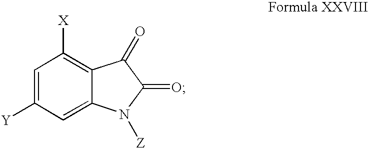
wherein,
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and provided that when X is NH₂ and Y is H, or when Y is NH₂ and X is —OH, W and Z are not both H;
and provided that Z is not ribose;
and wherein at least one of X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
or a pharmaceutically acceptable acid, base, salt, polymorph, solvate, ester, tautomer, stereoisomer or prodrug thereof.

52. A compound of Formula A; 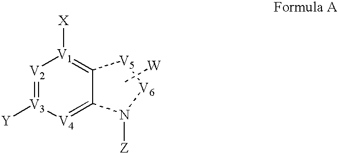
wherein each V is independently C or N, wherein if V₂ or V₄ is C said C is substituted only with hydrogen, and wherein each of V₅ and V₆ is unsubstituted or is independently substituted with one or more substitutents independently selected from W,
and wherein
W is H, F, Cl, Br, I, —OH, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B} or —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, heteroarylalkyl, —NR₁, R₂—OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
X is H, F, Cl, Br, I, NR₁R₂, —OH, SR₁, SOR₁, SO₂R₁, OR₁, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Y is H, F, Cl, Br, I, NR₁R₂, —OH, OR₁, CN, COOR₁, CONR₁R₂, C₁₋₆ alkyl, C₂₋₆ alkenyl, or C₂₋₆ alkynyl, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
Z is H, SR₁, SOR₁, SO₂R₁, OR₁, COOR₁, CONR₁R₂, —CN, C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B}, cycloalkyl, heteroalkyl, heterocycloalkyl, aryl, heteroaryl, alkylaryl, arylalkyl, alkylheteroaryl, heteroarylalkyl, NR₁R₂, —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
R₁ and R₂ are independently selected from the group consisting of H, COOR_{B}, CON(R_{C})₂ C₁₋₆ alkyl, C₂₋₆ alkenyl, C₂₋₆ alkynyl, —R_{A}OR_{B}—, —R_{A}NR_{B}, —R_{A}NR₁R_{B}, —R_{A}SR_{B}, —R_{A}SOR_{B} or —R_{A}SO₂R_{B} cycloalkyl, heteroalkyl, heterocycloalkyl, aryl, heteroaryl, alkylaryl, arylalkyl, alkylheteroaryl, and heteroarylalkyl;
each R_{A} is independently C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, alkylheteroarylalkyl, or heteroarylalkyl; and
each R_{B} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, —SO₂OH, —SO₂N(R_{A})₂, —SO₂NHR_{A} or —SO₂NH₂;
each R_{C} is independently H, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, or heteroarylalkyl;
and wherein any of said C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl may contain at least one substituent selected from H, F, Cl, Br, I, C₁₋₆ alkyl, C₂₋₆ alkenyl, C₂₋₆ alkynyl, cycloalkyl, heteroalkyl, heterocycloalkyl, aryl, heteroaryl, alkylaryl, arylalkyl, alkylheteroaryl, heteroarylalkyl, NR₅R₆, —N(R₈)SO₂NR₅R₆, —N(R₈)SO₂OH or —OSO₂NR₅R₆, and wherein when more than one said substituent is present said substituents may be fused to form one or more additional ring systems;
and provided that when X is NH₂ and Y is H, or when Y is NH₂ and X is —OH, W and Z are not both H;
and wherein at least one of W, X, Y or Z comprises a substituent selected from —OSO₂N(R_{C})₂, —N(R_{C})SO₂OH, —N(R_{C})SO₂R_{C}, —R_{A}OSO₂N(R_{C})₂, or —R_{A}N(R_{C})OSO₂R_{C};
including any pharmaceutically acceptable acid, base, salt, polymorph, solvate, ester, tautomer, enantiomer, diastereomer or prodrug thereof.
